SEQUENCE LISTING (144 sequences)

SEQ ID NO: 1 - protein, 29 aa, synthetic construct
  REGION 1..29: note = Description of Artificial Sequence: Synthetic peptide
KQSLRLGLSRLARVKRLHPGARRRRRRRR

SEQ ID NO: 2 - protein, 29 aa, synthetic construct
  REGION 1..29: note = Description of Artificial Sequence: Synthetic peptide
KQSLRLGQSRLARVKRLHPGARRRRRRRR

SEQ ID NO: 3 - protein, 29 aa, synthetic construct
  REGION 1..29: note = Description of Artificial Sequence: Synthetic peptide
KQSLRLGLSRLARVKRLHPGARRRRRRRR

SEQ ID NO: 4 - protein, 31 aa, synthetic construct
  REGION 1..31: note = Description of Artificial Sequence: Synthetic polypeptide
KQSNLRLGLSRLARVKRLHPGCCRRRRRRRR

SEQ ID NO: 5 - protein, 21 aa, synthetic construct
  REGION 1..21: note = Description of Artificial Sequence: Synthetic peptide
KRRRRRRRKRLGVRLRVSRML

SEQ ID NO: 6 - protein, 21 aa, synthetic construct
  REGION 1..21: note = Description of Artificial Sequence: Synthetic peptide
KRRRRRRRKRLGQRLRQSRML

SEQ ID NO: 7 - protein, 16 aa, synthetic construct
  REGION 1..16: note = Description of Artificial Sequence: Synthetic peptide
KRRRRRRRRLGMSRRF

SEQ ID NO: 8 - skipped

SEQ ID NO: 9 - protein, 21 aa, synthetic construct
  REGION 1..21: note = Description of Artificial Sequence: Synthetic peptide
KRRRRRRRKRLGLRLGVSRRV

SEQ ID NO: 10 - protein, 20 aa, synthetic construct
  REGION 1..20: note = Description of Artificial Sequence: Synthetic peptide
LMRSVRLRVGLRKRRRRRRR

SEQ ID NO: 11 - protein, 26 aa, synthetic construct
  REGION 1..26: note = Description of Artificial Sequence: Synthetic peptide
KRRRRRRRSLRLGLSRLARVKRLHPG

SEQ ID NO: 12 - skipped

SEQ ID NO: 13 - protein, 13 aa, Human immunodeficiency virus 1
GRKKRRQRRRPPQ

SEQ ID NO: 14 - protein, 8 aa, synthetic construct
  REGION 1..8: note = Description of Artificial Sequence: Synthetic peptide
RRRRRRRR

SEQ ID NO: 15 - protein, 18 aa, unidentified
  REGION 1..18: note = Description of Unknown: MAP sequence
KLALKLALKALKAALKLA

SEQ ID NO: 16 - protein, 27 aa, unidentified
  REGION 1..27: note = Description of Unknown: Transportan sequence
GWTLNSAGYLLGKINLKALAALAKKIL

SEQ ID NO: 17 - protein, 18 aa, unidentified
  REGION 1..18: note = Description of Unknown: Pegelin sequence
RGGRLSYSRRRFSTSTGR

SEQ ID NO: 18 - protein, 16 aa, Drosophila sp.
RQIKIWFQNRRMKWKK

SEQ ID NO: 19 - protein, 8 aa, synthetic construct
  REGION 1..8: note = Description of Artificial Sequence: Synthetic peptide
  VARIANT 4: note = MOD_RES - Leu, Met or Val
  VARIANT 7: note = MOD_RES - Arg, Leu or Lys
  VARIANT 8: note = MOD_RES - Arg, Phe or Val
RLGXSRXX

SEQ ID NO: 20 - protein, 258 aa, Vibrio cholerae
MVKIIFVFFIFLSSFSYANDDKLYRADSRPPDEIKQSGGLMPRGQSEYFDRGTQMNINLYDHARGTQTGFVRHDDGYVSTSISLRSAHLVGQTILSGHSTYYIYVIATAPNMFNVNDVLGAYSPHPDEQEVSALGGIPYSQIYGWYRVHFGVLDEQLHRNRGYRDRYYSNLDIAPAADGYGLAGFPPEHRAWREEPWIHHAPPGCGNAPRSSMSNTCDEKTQSLGVKFLDEYQSKVKRQIFSGYQSDIDTHNRIKDEL

SEQ ID NO: 21 - protein, 593 aa, Salmonella enterica
MLILNGFSSATLALITPPFLPKGGKALSQSGPDGLASITLPLPISAERGFAPALALHYSSGGGNGPFGVGWSCATMSIARRTSHGVPQYNDSDEFLGPDGEVLVQTLSTGDAPNPVTCFAYGDVSFPQSYTVTRYQPRTESSFYRLEYWVGNSNGDDFWLLHDSNGILHLLGKTAAARLSDPQAASHTAQWLVEESVTPAGEHIYYSYLAENGDNVDLNGNEAGRDRSAMRYLSKVQYGNATPAADLYLWTSATPAVQWLFTLVFDYGERGVDPQVPPAFTAQNSWLARQDPFSLYNYGFEIRLHRLCRQVLMFHHFPDELGEADTLVSRLLLEYDENPILTQLCAARTLAYEGDGYRRAPVNNMMPPPPPPPPPMMGGNSSRPKSKWAIVEESKQIQALRYYSAQGYSVINKYLRGDDYPETQAKETLLSRDYLSTNEPSDEEFKNAMSVYINDIAEGLSSLPETDHRVVYRGLKLDKPALSDVLKEYTTIGNIIIDKAFMSTSPDKAWINDTILNIYLEKGHKGRILGDVAHFKGEAEMLFPPNTKLKIESIVNCGSQDFASQLSKLRLSDDATADTNRIKRIINMRVLNS

SEQ ID NO: 22 - protein, 601 aa, Mycoplasma pneumoniae
MSENLYFQGHMPNPVRFVYRVDLRSPEEIFEHGFSTLGDVRNFFEHILSTNFGRSYFISTSETPTAAIRFFGSWLREYVPEHPRRAYLYEIRADQHFYNARATGENLLDLMRQRQVVFDSGDREMAQMGIRALRTSFAYQREWFTDGPIAAANVRSAWLVDAVPVEPGHAHHPAGRVVETTRINEPEMHNPHYQELQTQANDQPWLPTPGIATPVHLSIPQAASVADVSEGTSASLSFACPDWSPPSSNGENPLDKCIAEKIDNYNLQSLPQYASSVKELEDTPVYLRGIKTQKTFMLQADPQNNNVFLVEVNPKQKSSFPQTIFFWDVYQRICLKDLTGAQISLSLTAFTTQYAGQLKVHLSVSAVNAVNQKWKMTPQDIAITQFRVSSELLGQTENGLFWNTKSGGSQHDLYVCPLKNPPSDLEELQIIVDECTTHAQFVTMRAASTFFVDVQLGWYWRGYYYTPQLSGWSYQMKTPDGQIFYDLKTSKIFFVQDNQNVFFLHNKLNKQTGYSWDWVEWLKHDMNEDKDENFKWYFSRDDLTIPSVEGLNFRHIRCYADNQQLKVIISGSRWGGWYSTYDKVESNVEDKILVKDGFDRF

SEQ ID NO: 23 - protein, 250 aa, Streptococcus pyogenes
MLKKRYQLAMILLLSCFSLVWQTEGLVELFVCEHYERAVCEGTPAYFTFSDQKGAETLIKKRWGKGLVYPRAEQEAMAAYTCQQAGPINTSLDKAKGKLSQLTPELRDQVAQLDAATHRLVIPWNIVVYRYVYETFLRDIGVSHADLTSYYRNHQFNPHILCKIKLGTRYTKHSFMSTTALKNGAMTHRPVEVRICVKKGAKAAFVEPYSAVPSEVELLFPRGCQLEVVGAYVSQDHKKLHIEAYFKGSL

SEQ ID NO: 24 - protein, 264 aa, Pseudomonas syringae
MNINRQLPVSGSERLLTPDVGVSRQACSERHYSTGQDRHDFYRFAARLHVDAQCFGLSIDDLMDKFSDKHFRAEHPEYRDVYPEECSAIYMHTAQDYSSHLVRGEIGTPLYREVNNYLRLQHENSGREAEIDNHDEKLSPHIKMLSSALNRLMDVAAFRGTVYRGIRGDLDTIARLYHLFDTGGRYVEPAFMSTTRIKDSAQVFEPGTPNNIAFQISLKRGADISGSSQAPSEEEIMLPMMSEFVIEHASALSEGKHLFVLSQI

SEQ ID NO: 25 - protein, 601 aa, Vibrio cholerae
MKTIISLIFIMFPLFVSAHNGNFYRADSRSPNEIKDLGGLYPRGYYDFFERGTPMSISLYDHARGAPSGNTRYDDGFVSTTTDIDSAHEIGQNILSGYTEYYIYLIAPAPNLLDVNAVLGRYSPHPQENEYSALGGIPWTQVIGWYVVNNGVLDRNIHRNRQFRADLFNNLSPALPSESYQFAGFEPEHPAWRQEPWINFAPPGCGRNVRLTKHINQQDCSNSQEELVYKKLQDLRTQFKVDKKLKLVNKTSSNNIIFPNHDFIREWVDLDGNGDLSYCGFTVDSDGSRKRIVCAHNNGNFTYSSINISLSDYGWPKGQRFIDANGDGLVDYCRVQYVWTHLYCSLSLPGQYFSLDKDAGYLDAGYNNSRAWAKVIGTNKYSFCRLTSNGYICTDIDSYSTAFKDDDQGWADSRYWMDIDGNGGDDYCRLVYNWTHLRCNLQGKDGLWKRVESKYLDGGYPSLRFKIKMTSNKDNYCRIVRNHRVMECAYVSDNGEFHNYSLNMPFSLYNKNDIQFIDIDGDNRDDICRYNSAPNTMECYLNQDKSFSQNKLVLYLSAKPISSLGSGSSKIIRTFNSEKNSSAYCYNAGYGTLRCDEFVIY

SEQ ID NO: 26 - protein, 476 aa, Bacillus cereus
MKEIIRNLVRLDVRSDVDENSKKTQELVEKLPHEVLELYKNVGGEIYITDKRLTQHEELSDSSHKDMFIVSSEGKSFPLREHFVFAKGGKEPSLIIHAEDYASHLSSVEVYYELGKAIIRDTFPLNQKELGNPKFINAINEVNQQKEGKGVNAKADEDGRDLLFGKELKKNLEHGQLVDLDLISGNLSEFQHVFAKSFALYYEPHYKEALKSYAPALFNYMLELDQMRFKEISDDVKEKNKNVLDFKWYTRKAESWGVQTFKNWKENLTISEKDIITGYTGSKYDPINEYLRKYDGEIIPNIGGDLDKKSKKALEKIENQIKNLDAALQKSKITENLIVYRRVSELQFGKKYEDYNLRQNGIINEEKVMELESNFKGQTFIQHNYMSTSLVQDPHQSYSNDRYPILLEITIPEGVHGAYIADMSEYPGQYEMLINRGYTFKYDKFSIVKPTREEDKGKEYLKVNLSIYLGNLNREK

SEQ ID NO: 27 - protein, 487 aa, Enterococcus faecalis
  source 1..487: note = Enterococcus faecalis V583
MSQLNKWQKELQALQKANYQETDNQLFNVYRQSLIDIKKRLKVYTENAESLSFSTRLEVERLFSVADEINAILQLNSPKVEKTIKGYSAKQAEQGYYGLWYTLEQSQNIALSMPLINHDYIMNLVNAPVAGKRLSKRLYKYRDELAQNVTNNIITGLFEGKSYAEIARWINEETEASYKQALRIARTEAGRTQSVTTQKGYEEAKELGINIKKKWLATIDKHTRRTHQELDGKEVDVDEEFTIRGHSAKGPRMFGVASEDVNCRCTTIEVVDGISPELRKDNESKEMSEFKSYDEWYADRIRQNESKPKPNFTELDFFGQSDLQDDSDKWVAGLKPEQVNAMKDYTSDAFAKMNKILRNEKYNPREKPYLVNIIQNLDDAISKFKLKHDIITYRGVSANEYDAILNGNVFKEFKSTSINKKVAEDFLNFTSANKDGRVVKFLIPKGTQGAYIGTNSSMKKESEFLLNRNLKYTVEIVDNILEVTILG

SEQ ID NO: 28 - protein, 457 aa, Pseudomonas aeruginosa
MHIQSSQQNPSFVAELSQAVAGRLGQVEARQVATPREAQQLAQRQEAPKGEGLLSRLGAALARPFVAIIEWLGKLLGSRAHAATQAPLSRQDAPPAASLSAAEIKQMMLQKALPLTLGGLGKASELATLTAERLAKDHTRLASGDGALRSLATALVGIRDGSLIEASRTQAARLLEQSVGGIALQQWGTAGGAASQHVLSASPEQLREIAVQLHAVMDKVALLRHAVESEVKGEPVDKALADGLVEHFGLEAEQYLGEHPDGPYSDAEVMALGLYTNGEYQHLNRSLRQGRELDAGQALIDRGMSAAFEKSGPAEQVVKTFRGTQGRDAFEAVKEGQVGHDAGYLSTSRDPSVARSFAGLGTITTLFGRSGIDVSEISIEGDEQEILYDKGTDMRVLLSAKDGQGVTRRVLEEATLGERSGHSEGLLDALDLATGTDRSGKPQEQDLRLRMRGLDLA

SEQ ID NO: 29 - protein, 265 aa, unidentified
  REGION 1..265: note = Description of Unknown: CdtB toxin sequence
MKKIICLFLSFNLAFANLENFNVGTWNLQGSSAATESKWSVSVRQLVSGANPLDILMIQEAGTLPRTATPTGRHVQQGGTPIDEYEWNLGTLSRPDRVFIYYSRVDVGANRVNLAIVSRMQAEEVIVLPPPTTVSRPIIGIRNGNDAFFNIHALANGGTDVGAIITAVDAHFANMPQVNWMIAGDFNRDPSTITSTVDRELANRIRVVFPTSATQASGGTLDYAITGNSNRQQTYTPPLLAAILMLASLRSHIVSDHFPVNFRKF

SEQ ID NO: 30 - protein, 560 aa, Corynebacterium diphtheriae
MSRKLFASILIGALLGIGAPPSAHAGADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 31 - protein, 621 aa, unidentified
  REGION 1..621: note = Description of Unknown: ExoU/VipB sequence
MKLAEIMTKSRKLKRNLLEISKTEAGQYSVSAPEHKGLVLSGGGAKGISYLGMIQALQERGKIKNLTHVSGASAGAMTASILAVGMDIKDIKKLIEGLDITKLLDNSGVGFRARGDRFRNILDVIYMMQMKKHLESVQQPIPPEQQMNYGILKQKIALYEDKLSRAGIVINNVDDIINLTKSVKDLEKLDKALNSIPTELKGAKGEQLENPRLTLGDLGRLRELLPEENKHLIKNLSVVVTNQTKHELERYSEDTTPQQSIAQVVQWSGAHPVLFVPGRNAKGEYIADGGILDNMPEIEGLDREEVLCVKAEAGTAFEDRVNKAKQSAMEAISWFKARMDSLVEATIGGKWLHATSSVLNREKVYYNIDNMIYINTGEVTTTNTSPTPEQRARAVKNGYDQTMQLLDSHKQTFDHPLMAILYIGHDKLKDALIDEKSEKEIFEASAHAQAILHLQEQIVKEMNDGDYSSVQNYLDQIEDILTVDAKMDDIQKEKAFALCIKQVNFLSEGKLETYLNKVEAEAKAAAEPSWATKILNLLWAPIEWVVSLFKGPAQDFKVEVQPEPVKVSTSENQETVSNQKDINPAVEYRKIIAEVRREHTDPSPSLQEKERVGLSTTFGGH

SEQ ID NO: 32 - protein, 211 aa, unidentified
  REGION 1..211: note = Description of Unknown: HopPtoE sequence
MNRVSGSSSATWQAVNDLVEQVSERTTLSTTGYQTAMGRLNKPEKSDADALMTMRRAQQYTDSAKRTYISETLMNLADLQQRKIYRTNSGNLRGAIEMTPTQLTDCVQKCREEGFSNCDIQALEIGLHLRHKLGISDFTIYSNRKLSHNYVVIHPSNAFPKGAIVDSWTGQGVVELDFKTRLKFKHREENYAVNANMHEWIERYGQAHVID

SEQ ID NO: 33 - protein, 204 aa, unidentified
  REGION 1..204: note = Description of Unknown: HopPtoF sequence
MGNICGTSGSRHVYSPSHTQRITSAPSTSTHVGGDTLTSIHQLSHSQREQFLNMHDPMRVMGLDHDTELFRTTDSRYIKNDKLAGNPQSMASILMHEELRPNRFASHTGAQPHEARAYVPKRIKATDLGVPSLNVMTGSLARDGIRAYDHMSDNQVSVKMRLGDFLERGGKVYADASSVADDGETSQALIVTLPKGQKVPVERV

SEQ ID NO: 34 - protein, 493 aa, unidentified
  REGION 1..493: note = Description of Unknown: HopPtoG sequence
MQIKNSHLYSASRMVQNTFNASPKMEVTNAIAKNNEPAALSATQTAKTHEGDSKGQSSNNSKLPFRAMRYAAYLAGSAYLYDKTANNFFLSTTSLHDGKGGFTSDARLNDAQDKARKRYQNNHSSTLENKNSLLSPLRLCGENQFLTMIDYRAATKIYLSDLVDTEQAHTSILKNIMCLKGELTNEEAIKKLNPEKTPKDYDLTNSEAYISKNKYSLTGVKNEETGSTGYTSRSITKPFVEKGLKHFIKATHGEKALTPKQCMETLDNLLRKSITLNSDSQFAAGQALLVFRQVYAGEDAWGDAERVILKSHYNRGTVLQDEADKIELSRPFSEQDLAKNMFKRNTSIAGPVLYHAYIYIQEKIFKLPPDKIEDLKHKSMADLKNLPLTHVKLSNSGVGFEDASGLGDSFTALNATSCVNHARIMSGEPPLSKDDVVILIGCLNAVYDNSSGIRHSLREIARGCFVGAGFTVQDGDDFYKQICKNASKQFYNG

SEQ ID NO: 35 - protein, 530 aa, Vibrio cholerae
MFKISVSQQANVMSTSDTAQRSSLKISIKSICNKSLSKKLHTLAEKCRRFSQELKEHTASKKQIVEQATTTVRESSLTKSDSELGSSRSLLTSDVLSSSSSHEDLTAVNLEDNDSVFVTIESSSELIVKQDGSIPPAPPLPGNIPPAPPLPSAGNIPTAPGLPKQKATTESVAQTSDNRSKLMEEIRQGVKLRATPKSSSTEKSASDPHSKLMKELINHGAKLKKVSTSDIPVPPPLPAAFASKPTDGRSALLSEIAGFSKDRLRKAGSSETLNVSQPTVAESSIPEAYDLLLSDEMFNLSPKLSETELNTLADSLADYLFKAADIDWMQVIAEQTKGSTQATSLKSQLEQAPEYVKAFCDEILKFPDCYKSADVASPESPKAGPSSVIDVALKRLQAGRNRLFSTIDAKGTNELKKGEAILESAINAARSVMTAEQKSALLSSNVKSATFKVFSELPCMEGFAEQNGKAAFNALRLAFYSSIQSGDTAQQDIARFMKENLATGFSGYSYLGLTSRVAQLEAQLAALTTK

SEQ ID NO: 36 - protein, 288 aa, Yersinia pestis
MIGPISQINISGGLSEKETSSLISNEELKNIITQLETDISDGSWFHKNYSRMDVEVMPALVIQANNKYPEMNLNLVTSPLDLSIEIKNVIENGVRSSRFIINMGEGGIHFSVIDYKHINGKTSLILFEPANFNSMGPAMLAIRTKTAIERYQLPDCHFSMVEMDIQRSSSECGIFSFALAKKLYIERDSLLKIHEDNIKGILSDGENPLPHDKLDPYLPVTFYKHTQGKKRLNEYLNTNPQGVGTVVNKKNETIVNRFDNNKSIVDGKELSVSVHKKRIAEYKTLLKV

SEQ ID NO: 37 - protein, 579 aa, unidentified
  REGION 1..579: note = Description of Unknown: AvrPtoB sequence
MAGINGAGPSGAYFVGHTDPEPASGGAHGSSSGASSSNSPRLPAPPDAPASQARDRREMLLRARPLSRQTREWVAQGMPPTAEAGVPIRPQESAEAAAPQARAEERHTPEADAAASHVRTEGGRTPQALAGTSPRHTGAVPHANRIVQQLVDAGADLAGINTMIDNAMRRHAIALPSRTVQSILIEHFPHLLAGELISGSELATAFRAALRREVRQQEASAPPRTAARSSVRTPERSTVPPTSTESSSGSNQRTLLGRFAGLMTPNQRRPSSASNASASQRPVDRSPPRVNQVPTGANRVVMRNHGNNEADAALQGLAQQGVDMEDLRAALERHILHRRPIPMDIAYALQGVGIAPSIDTGESLMENPLMNLSVALHRALGPRPARAQAPRPAVPVAPATVSRRPDSARATRLQVIPAREDYENNVAYGVRLLSLNPGAGVRETVAAFVNNRYERQAVVADIRAALNLSKQFNKLRTVSKADAASNKPGFKDLADHPDDATQCLFGEELSLTSSVQQVIGLAGKATDMSESYSREANKDLVFMDMKKLAQFLAGKPEHPMTRETLNAENIAKYAFRIVP

SEQ ID NO: 38 - protein, 1116 aa, unidentified
  REGION 1..1116: note = Description of Unknown: SdbA sequence
MHKKYNYYSLEKEKKTFWQHILDILKAPFRLPGWVVSFFLARNITHVALNPNNIPQQRLIHLTKTSNRPEDDIVVINFKKRPPHKWFNDTLIKIANTIAALPFVTPRLRTRLHYDNENDINHVNKLLAEIDALVQGKSKQKYCKGRAFDWSKIHLKGLEFLDPKMRGYVYEQLHEKYGYVSYTTKRKPNIEFFTLKTPDGSELDSVQVTGEDEEKKPMGERKFIITCIARDQNFINWIKDLNYTAKNLGATAISFNYRGVDYSRGLVWTENNLVDDILAQVQRLISLGADPKNICLDGMCIGGAVATIAAAKLHEKGMKVKLNNERSFTSLSSLVFGFIVPELQTANWWSPLTYGRFLLAGVVYALLTPLIWLAGWPVDVTKAWNRIPAQDKMYSVVRDKDNGLYDGVIHDHFCSIASLVDSQINSILYKLSTDQPLTEEEKQILCDDQFSHHFKPSQSVLKNPKYKGPHFISRQDLVAELGHREEYTNHDYFLDRLREKFQLDRATRPVALAEDGEKDIDGISSQLSNNKERPLIIASSGGTGHISATHGIINDLQSKTDNVVITQHHAELYKNKPFSITSVLIRIGVWFTSLPILEDILKGVMRFIGYPVLPSSSIFWDQMSKIQQSETKKENGIETGRTRPYVDMLLDIYPEGYEYTAFNNATHLTSSIEDIQTMISFKGHVEEDNRNIVYQNILQRLMHAAKQNTPYTRLISTQALSLGAICDAVKYYNTVFLPVYNAERGTSYQPIAIDQYMTDLPSLGCIHFMNNLEELTSEQRQLMEIHAVNMSEPFKEAHFGKEQGFKAVHNIDPRNNPMIRNAFKDPSLTKYLDKTQSFDLHFNVYKKEKQNALPVLNGKEKITIKPHAKIASIMIGSLAANASADYAKYLLNQGYEHIFLFGGLNDSIAARIDQIINSYPAPTRDEIRKKIILLGNQSDVEMAPIMTRSNCVVIRGGGLSVMEQMAMPIMDDKIVLLHHEDNEEGPLTSGLSWEDGNSDKLIEYLSEKGAYAKKTSPGLCSGHLHEAEKSFEKKYHGQLKSTETKKKVDLTIPQQETYSLKKEWDRKTGYTESGHILSHQHRFFNTIPEVREPFCSKEDLHHNELSSQSLVSVSAG

SEQ ID NO: 39 - protein, 965 aa, unidentified
  REGION 1..965: note = Description of Unknown: SidG sequence
MSRSKDEVLEANDSLFGITVQTWGTNDRPSNGMMNFADQQFFGGDVGHASINMKLPVTDKTKQWIEKYCYSQTYDQFKKVKGNEDKTYEEYLKTAKRLIPVELKTQVTRKAQYDSNGNLVTTHEKAYEQIYFDIDWSWWPGRLQNTEDDMVWEREGKHFEYDEKWKEYLQPEQRVHRGKLGSRKMDYAPTSIIHQRDIPTSELEKITRDHKIHTIEEKLNVVKLLQSKIDEMPHTKMSPSMELMFKNLGINVEKLLDETKDNGVDPTNLEAMREYLTNRLTERKLELETELSEAKKEVDSTQVKNKVEDVYYDFEYKLNQVRKKMEEVNSQLEKMDSLLHKLEGNTSGPIPYTAEIDELMSVLPFLKEELELENGTLSPKSIENLIDHIDELKNELASKQEKKNERNLNLIKKYEELCEQYKDDEEGLEEALWEEGIDVEEVNSAKKDISKPAPEIQKLTDLQEQLRNHKESGVKLSSELEETLNSSVKMWKTKIDSPCQVISESSVKALVSKINSTRPELVKEKEQLPEQEESLSKEAKKAQEELIKIQEFSQFYSENSSAYMVIGLPPHHQVSLPLAVNGKRGLHPEAMLKKMHELVAGPEKKEFNLHTNNCSLTSIEVLSAGAQHDPLLHSIMGTRALGFFGTPQQVLENAKLTSKTINEGKKSNIFTPLVTASPLDRALGYAMSIYMDPEASKAKQNAGLALGVLVGLAKTPGIIIGSLLNPKQGFNDILNTLNLVYSRNSTGLKVGLTLMALPAMIVLAPLAAIQKGVEVIAETIAKPFKLIANLFKQKPESTDEITVSVGSKKVAEKEGSYSNTALAGLVNSKIKSKIDENTITVEFQKSPQKMIEEFESQLKENPGKVVVLSEKAHNAVLKFVSKSDDEALKQKFYDCCNQSVARSQKFAPKTRDEIDELVEEVTSTDKTELTTSPRQEPSMSSTIDEEENIDSEHQIETGTESTMRI

SEQ ID NO: 40 - protein, 665 aa, unidentified
  REGION 1..665: note = Description of Unknown: VpdA sequence
MKTKQEVSQQDKLKDSKSSTPLQTKETWFISDALNITFDPYDFSISVTEQAPMPYRIVFSGGGSRILAHIGALDELTRHGLKFTEFSGSSAGAMVAAFAYLGYNCSEIKQIISWFNEDKLLDSPLIFNFNNIKQIFNKGGLSSAKLMRQAANYVILKKVMDIISDEKFKTRFAKFQNFLEENIYRCPENITFQTLARIKEICPECELGEKLFITGTNLSTQKHEVFSIDTTPSMALADAIIISANLPIAFERICYQGNVYSDGGISNNLPAHCFSEKGHKTTFLKHKDDVDFSVLALQFDNGLEENALYSQNPIPKWSWLSNTFYSLITGHPNVTENWYEDLQILRRHAHQSILIKTPTIALTNLTISQDTKKALVESGRTAAKTYLELHEFYTDDYGNIRHNECLHEKFQKPEELLDYCVLHSHFELLKKIKQAISCSQYLEKGYKHYLCELCDNLLPPQLKCPNEGSGTEQPEIKLEKDTIICEKNNNSGLTFSMTFFGVPSPLVKTLNQDSPELKIKLFTGLYPILIQNWQNLCPVSGISGILNSIRMSFVEISSTDTCIKTLIDKLNEIEIGHFLIFVFKAALKNYDKHDFILLLKNLKHLHHSIELIRNKPFHSDDRFYGQWSFEGHDPKRILEFIKSDDISGLMTILEDKKALPNNKPN

SEQ ID NO: 41 - protein, 246 aa, Legionella pneumophila
MVSLEHIQKLISECRKLGKDGLDNGTNGLIPELEIDVVPPSAFLGVGNNPAIFVNSKTYKLMRTTHEKWVENKTIVFKSYLLSQPAIKIIGAIVHETGHAFNVAAKIPNTEANACIFEIEVLMRLFQVKSPLLLGCTELDMQSYFKSRLTDYNKCVKDCQCLAEMVEFITHQFKLDEVSISEKENQIPLLSISNKWPGLFAKKQIAPDMDKLLTSPVTITPEVKILFYQLVKEHFHSPETEIKLDI

SEQ ID NO: 42 - protein, 644 aa, Legionella pneumophila
MYKIYSYLGWRIDMKTENLPQAGQEAQIDKKIHFIWVGHIMPQKNIQVVSEWAEKNPGYETIIWVDKKIAPAKELDLFILDMKSKGITVKDINEEGVCRDSIRHELDQESPNYGMVSDMLRLNILAAEGGIYLDSDILCSAPFPDEIYAPFGFLLSPWSQGANNTLCNDIILCSKGNQIIQQLADAIEQSYIARDSFEFTHEYASMKETKGERIAKTLGVTGPGFLFHQLKKMGILNDKSEMEAIHWELQDQRYLIDGSVKEPDYFYVPQNNTNDASWVPSIKRPGIENMSFQERLENAVQLIAFDIQKTGLFNLDHYANELKVKQNSWCIAAETSPELKPDSYLLIRPRDKTGEWTLYYVDEDKKLNPVTLPVIKGAIKLSEVSDPLRKFHTLLSQVSDPVNPTAHELKQIGRALIELKPRQDEWHCKNKWSGAEEIAQELWQRITSNETLRAQIKQCFTQFESLKPRVAELGLTRASGAGTEVEAHESTVKEQEIISQNTVGEEGTKEKNSVQLASENSSDEKIKTAHDLIDEIIQDVIQLDGKLGLLGGNTRQLEDGRVINIPNGAAMIFDDYKKYKQGELTAESALESMIKIAKLSNQLNRHTFFNQRQPETGQFYKKVAAIDLQTTIAAEYDNNHGLRI

SEQ ID NO: 43 - protein, 219 aa, Yersinia enterocolitica
MKISSFISTSLPLPTSVSGSSSVGEMSGRSVSQQTSDQYANNLAGRTESPQGSSLASRIIERLSSVAHSVIGFIQRMFSEGSHKPVVTPAPTPAQMPSPTSFSDSIKQLAAETLPKYMQQLNSLDAEMLQKNHDQFATGSGPLRGSITQCQGLMQFCGGELQAEASAILNTPVCGIPFSQWGTIGGAASAYVASGVDLTQAANEIKGLAQQMQKLLSLM

SEQ ID NO: 44 - protein, 543 aa, Salmonella sp.
  source 1..543: note = Salmonella typhimurium
MLKYEERKLNNLTLSSFSKVGVSNDARLYIAKENTDKAYVAPEKFSSKVLTWLGKMPLFKNTEVVQKHTENIRVQDQKILQTFLHALTEKYGETAVNDALLMSRINMNKPLTQRLAVQITECVKAADEGFINLIKSKDNVGVRNAALVIKGGDTKVAEKNNDVGAESKQPLLDIALKGLKRTLPQLEQMDGNSLRENFQEMASGNGPLRSLMTNLQNLNKIPEAKQLNDYVTTLTNIQVGVARFSQWGTCGGEVERWVDKASTHELTQAVKKIHVIAKELKNVTAELEKIEAGAPMPQTMSGPTLGLARFAVSSIPINQQTQVKLSDGMPVPVNTLTFDGKPVALAGSYPKNTPDALEAHMKMLLEKECSCLVVLTSEDQMQAKQLPPYFRGSYTFGEVHTNSQKVSSASQGEAIDQYNMQLSCGEKRYTIPVLHVKNWPDHQPLPSTDQLEYLADRVKNSNQNGAPGRSSSDKHLPMIHCLGGVGRTGTMAAALVLKDNPHSNLEQVRADFRDSRNNRMLEDASQFVQLKAMQAQLLMTTAS

SEQ ID NO: 45 - protein, 240 aa, Salmonella sp.
  source 1..240: note = Salmonella typhimurium
MTNITLSTQHYRIHRSDVEPVKEKTTEKDIFAKSITAVRNSFISLSTSLSDRFSLHQQTDIPTTHFHRGNASEGRAVLTSKTVKDFMLQKLNSLDIKGNASKDPAYARQTCEAILSAVYSNNKDQCCKLLISKGVSITPFLKEIGEAAQNAGLPGEIKNGVFTPGGAGANPFVVPLIASASIKYPHMFINHNQQVSFKAYAEKIVMKEVTPLFNKGTMPTPQQFQLTIENIANKYLQNAS

SEQ ID NO: 46 - protein, 561 aa, Salmonella sp.
  source 1..561: note = Salmonella typhimurium
MQIQSFYHSASLKTQEAFKSLQKTLYNGMQILSGQGKAPAKAPDARPEIIVLREPGATWGNYLQHQKASNHSLHNLYNLQRDLLTVAATVLGKQDPVLTSMANQMELAKVKADRPATKQEEAAAKALKKNLIELIAARTQQQDGLPAKEAHRFAAVAFRDAQVKQLNNQPWQTIKNTLTHNGHHYTNTQLPAAEMKIGAKDIFPSAYEGKGVCSWDTKNIHHANNLWMSTVSVHEDGKDKTLFCGIRHGVLSPYHEKDPLLRHVGAENKAKEVLTAALFSKPELLNKALAGEAVSLKLVSVGLLTASNIFGKEGTMVEDQMRAWQSLTQPGKMIHLKIRNKDGDLQTVKIKPDVAAFNVGVNELALKLGFGLKASDSYNAEALHQLLGNDLRPEARPGGWVGEWLAQYPDNYEVVNTLARQIKDIWKNNQHHKDGGEPYKLAQRLAMLAHEIDAVPAWNCKSGKDRTGMMDSEIKREIISLHQTHMLSAPGSLPDSGGQKIFQKVLLNSGNLEIQKQNTGGAGNKVMKNLSPEVLNLSYQKRVGDENIWQSVKGISSLITS

SEQ ID NO: 47 - protein, 685 aa, Salmonella sp.
  source 1..685: note = Salmonella typhimurium
MVTSVRTQPPVIMPGMQTEIKTQATNLAANLSAVRESATTTLSGEIKGPQLEDFPALIKQASLDALFKCGKDAEALKEVFTNSNNVAGKKAIMEFAGLFRSALNATSDSPEAKTLLMKVGAEYTAQIIKDGLKEKSAFGPWLPETKKAEAKLENLEKQLLDIIKNNTGGELSKLSTNLVMQEVMPYIASCIEHNFGCTLDPLTRSNLTHLVDKAAAKAVEALDMCHQKLTQEQGTSVGREARHLEMQTLIPLLLRNVFAQIPADKLPDPKIPEPAAGPVPDGGKKAEPTGINININIDSSNHSVDNSKHINNSRSHVDNSQRHIDNSNHDNSRKTIDNSRTFIDNSQRNGESHHSTNSSNVSHSHSRVDSTTHQTETAHSASTGAIDHGIAGKIDVTAHATAEAVTNASSESKDGKVVTSEKGTTGETTSFDEVDGVTSKSIIGKPVQATVHGVDDNKQQSQTAEIVNVKPLASQLAGVENVKTDTLQSDTTVITGNKAGTTDNDNSQTDKTGPFSGLKFKQNSFLSTVPSVTNMHSMHFDARETFLGVIRKALEPDTSTPFPVRRAFDGLRAEILPNDTIKSAALKAQCSDIDKHPELKAKMETLKEVITHHPQKEKLAEIALQFAREAGLTRLKGETDYVLSNVLDGLIGDGSWRAGPAYESYLNKPGVDRVITTVDGLHMQR

SEQ ID NO: 48 - protein, 732 aa, Yersinia pestis
MKSVKIMGTMPPSISLAKAHERISQHWQNPVGELNIGGKRYRIIDNQVLRLNPHSGFSLFREGVGKIFSGKMFNFSIARNLTDTLHAAQKTTSQELRSDIPNALSNLFGAKPQTELPLGWKGEPLSGAPDLEGMRVAETDKFAEGESHISIIETKDKQRLVAKIERSIAEGHLFAELEAYKHIYKTAGKHPNLANVHGMAVVPYGNRKEEALLMDEVDGWRCSDTLRTLADSWKQGKINSEAYWGTIKFIAHRLLDVTNHLAKAGVVHNDIKPGNVVFDRASGEPVVIDLGLHSRSGEQPKGFTESFKAPELGVGNLGASEKSDVFLVVSTLLHCIEGFEKNPEIKPNQGLRFITSEPAHVMDENGYPIHRPGIAGVETAYTRFITDILGVSADSRPDSNEARLHEFLSDGTIDEESAKQILKDTLTGEMSPLSTDVRRITPKKLRELSDLLRTHLSSAATKQLDMGGVLSDLDTMLVALDKAEREGGVDKDQLKSFNSLILKTYRVIEDYVKGREGDTKNSSTEVSPYHRSNFMLSIVEPSLQRIQKHLDQTHSFSDIGSLVRAHKHLETLLEVLVTLSQQGQPVSSETYGFLNRLAEAKITLSQQLNTLQQQQESAKAQLSILINRSGSWADVARQSLQRFDSTRPVVKFGTEQYTAIHRQMMAAHAAITLQEVSEFTDDMRNFTVDSIPLLIQLGRSSLMDEHLVEQREKLRELTTIAERLNRLEREWM

SEQ ID NO: 49 - protein, 529 aa, Yersinia pestis
MFINPRNVSNTFLQEPLRHSSNLTEMPVEAENVKSKTEYYNAWSEWERNAPPGNGEQREMAVSRLRDCLDRQAHELELNNLGLSSLPELPPHLESLVASCNSLTELPELPQSLKSLQVENNNLKALPDLPPSLKKLHVRENDLTDLPELPQSLESLRVDNNNLKALSDLPPSLEYLTASSNKLEELPELQNLPFLAAIYADNNLLETLPDLPPSLKKLHVRENDLTDLPELPQSLESLQVDNNNLKALSDLPPSLEYLTASSNKLEELPELQNLPFLAAIYADNNLLETLPDLPPHLEILVASYNSLTELPELPQSLKSLRVDNNNLKALSDLPPSLEYLTASSNKLEELPELQNLPFLAAIYADNNLLETLPDLPPSLKKLHVRENDLTDLPELPQSLTFLDVSDNNISGLSELPPNLYYLDASSNEIRSLCDLPPSLVDLNVKSNQLSELPALPPHLERLIASFNYLAEVPELPQNLKQLHVEQNALREFPDIPESLEELEMDSERVVDPYEFAHETTDKLEDDVFE

SEQ ID NO: 50 - protein, 35 aa, unidentified
  REGION 1..35: note = Description of Unknown: Amatoxin sequence
MSDINATRLPIWGIGCNPCVGDDVTTLLTRGEALC

SEQ ID NO: 51 - protein, 34 aa, unidentified
  source 1..34: note = Amanita fuliginea
MSDINATRLPAWLVDCPCVGDDVNRLLTRGESLC

SEQ ID NO: 52 - protein, 291 aa, Ustilago maydis
MIKPERSILTILIGILCLLAYVLANGEPHDGDNEWSSYCSDQGFRRSDDGLVTTPDVGQESIGKNSINGSELVDYLQCLKVRLNGQKQVVSNDGWLLLLVQEPSVNVTQKAMSECNYNVSSGHKAGSYIQVTNTPADYKVISRRGSYEGDQLPEDVKPYFGVQKTSDYRPISKRINPNLTLRQLAYNFAALNMCSLWCNSCISRSCPYYIAELTVHVNNIHHGTVWLHHFCRNASPQGGNLYSTLTISHKDTAYYVGTGWWKVRSTAATTNDVAGDWYPASWNQYWCGPHY

SEQ ID NO: 53 - protein, 219 aa, Ustilago maydis
MLIFSVLMYLGLLLAGASALPNGLSPRNNAFCAGFGLSCKWECWCTAHGTGNELRYATAAGCGDHLSKSYYDARAGHCLFSDDLRNQFYSHCSSLNNNMSCRSLSKRTIQDSATDTVDLGAELHRDDPPPTASDIGKRGKRPRPVMCQCVDTTNGGVRLDAVTRAACSIDSFIDGYYTEKDGFCRAKYSWDLFTSGQFYQACLRYSHAGTNCQPDPQYE

SEQ ID NO: 54 - protein, 316 aa, Saccharomyces cerevisiae
MTKPTQVLVRSVSILFFITLLHLVVALNDVAGPAETAPVSLLPREAPWYDKIWEVKDWLLQRATDGNWGKSITWGSFVASDAGVVIFGINVCKNCVGERKDDISTDCGKQTLALLVSIFVAVTSGHHLIWGGNRPVSQSDPNGATVARRDISTVADGDIPLDFSALNDILNEHGISILPANASQYVKRSDTAEHTTSFVVTNNYTSLHTDLIHHGNGTYTTFTTPHIPAVAKRYVYPMCEHGIKASYCMALNDAMVSANGNLYGLAEKLFSEDEGQWETNYYKLYWSTGQWIMSMKFIEESIDNANNDFEGCDTGH

SEQ ID NO: 55 - protein, 296 aa, Saccharomyces cerevisiae
MGHLAILFSIIAVLNIATAVASSDSIYLKGHRVGQDIDSLYRVYDNGTMYPVTFNEWLNDLTGMNDLATNNATILKRDSSDVSCVTETCQYVDYHVDDEGVITIDISTYRIPVEWDSGSAGNASYGVSKRDTKYETFCKKKICGINVSGFCNAYDFAVHAFDFGGSVYNPVSGITDRIKEATKRDKTECLGYELDHVRIDPAVDWSISISTWKQGSANCDTQASADSLKCAAQKALESEHNHQKTAFCIHLDNGGSFNLDIRLISELSFSKYNPWALPCPKYKGSNSWQVVSDCFQ

SEQ ID NO: 56 - protein, 708 aa, Saccharomyces cerevisiae
MPRFAIIFALLIAYSLFLSTLFTGSIPDRANTVTSNAPCQVVIWDWIRTRRICNCCSRLCYSLLGRSNLSRTAKRGVCTIAGAVLATAAVIVAAVLVGKSSGSATKRGLTKTISVLNHTIPFTDHILNGQTLSNGTGSNFVTIGFSGYAVHATIKRASTTDIISWVIPESMEPTLARVASYVSSSSINLAAVPDTGGNASALSFQNAVQEFATSWVSMTYDQSYGDLRNVANDEGGEEILILMRKRSYRISFQVIETGSTALLLRTRRVVSQLITMTYLVTVQARVGIQIGDIFQHYGGIDNYVMTSISVLRTLEDKAFHENKLLIVREPPNKSNQDANQSYRLRPFSANDLIQNLKSVDIGFLAFCSFFDKYAHYPEIIMMKITIFISKGNLWSIIYVIQARYVRKRVMKVRGQMPGGLLTNMESLLNIVSTPNLNISEFHIQTHSMSQSKPMYFQKQCYSSQNNIIYIYNSIHITCGAVYVIVHDVRTPSVFVLIELRNCKPLKNSWCETTKTSPRDTKIKKNEYNETVCRRAGALLDGRVRTIRFLMMRTHWSRVKGVSCNTANRLSRFCNHVVSYYPSQNATIHLLPTSLRAESLEQQYTTRPLSSSNNRFCCLKSIFINNCKKACESPSLVSCNLQQTAELLMVYYLYICEACYVSRNHDLLSKQCMSTVRAVYVARMRLPKFRSTFPCMPRLCWLVNGVVVV

SEQ ID NO: 57 - protein, 768 aa, Bacillus anthracis
MHVKEKEKNKDENKRKDEERNKTQEEHLKEIMKHIVKIEVKGEEAVKKEAAEKLLEKVPSDVLEMYKAIGGKIYIVDGDITKHISLEALSEDKKKIKDIYGKDALLHEHYVYAKEGYEPVLVIQSSEDYVENTEKALNVYYEIGKILSRDILSKINQPYQKFLDVLNTIKNASDSDGQDLLFTNQLKEHPTDFSVEFLEQNSNEVQEVFAKAFAYYIEPQHRDVLQLYAPEAFNYMDKFNEQEINLSLEELKDQRMLSRYEKWEKIKQHYQHWSDSLSEEGRGLLKKLQIPIEPKKDDIIHSLSQEEKELLKRIQIDSSDFLSTEEKEFLKKLQIDIRDSLSEEEKELLNRIQVDSSNPLSEKEKEFLKKLKLDIQPYDINQRLQDTGGLIDSPSINLDVRKQYKRDIQNIDALLHQSIGSTLYNKIYLYENMNINNLTATLGADLVDSTDNTKINRGIFNEFKKNFKYSISSNYMIVDINERPALDNERLKWRIQLSPDTRAGYLENGKLILQRNIGLEIKDVQIIKQSEKEYIRIDAKVVPKSKIDTKIQEAQLNINQEWNKALGLPKYTKLITFNVHNRYASNIVESAYLILNEWKNNIQSDLIKKVTNYLVDGNGRFVFTDITLPNIAEQYTHQDEIYEQVHSKGLYVPESRSILLHGPSKGVELRNDSEGFIHEFGHAVDDYAGYLLDKNQSDLVTNSKKFIDIFKEEGSNLTSYGRTNEAEFFAEAFRLMHSTDHAERLKVQKNAPKTFQFINDQIKFIINS

SEQ ID NO: 58 - protein, 319 aa, unidentified
  REGION 1..319: note = Description of Unknown: Shiga Toxin sequence
MKCILLKWVLCLLLGFSSVSYSREFTIDFSTQQSYVSSLNSIRTEISTPLEHISQGTTSVSVINHTPPGSYFAVDIRGLDVYQARFDHLRLIIEQNNLYVAGFVNTATNTFYRFSDFAHISVPGVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVLPEYRGEDGVRVGRISFNNISAILGTVAVILNCHHQGARSVRAVNEESQPECQITGDRPVIKINNTLWESNTAAAFLNRKSQSLYTTGE

SEQ ID NO: 59 - protein, 565 aa, Ricinus communis
MYAVATWLCFGSTSGWSFTLEDNNIFPKQYPIINFTTAGATVQSYTNFIRAVRGRLTTGADVRHDIPVLPNRVGLPINQRFILVELSNHAELSVTLALDVTNAYVVGYRAGNSAYFFHPDNQEDAEAITHLFTDVQNRYTFAFGGNYDRLEQLAGNLRENIELGNGPLEEAISALYYYSTGGTQLPTLARSFIICIQMISEAARFQYIEGEMRTRIRYNRRSAPDPSVITLENSWGRLSTAIQESNQGAFASPIQLQRRNGSKFSVYDVSILIPIIALMVYRCAPPPSSQFSLLIRPVVPNFNADVCMDPEPIVRIVGRNGLCVDVRDGRFHNGNAIQLWPCKSNTDANQLWTLKRDNTIRSNGKCLTTYGYSPGVYVMIYDCNTAATDATRWQIWDNGTIINPRSSLVLAATSGNSGTTLTVQTNIYAVSQGWLPTNNTQPFVTTIVGLYGLCLQANSGQVWIEDCSSEKAEQQWALYADGSIRPQQNRDNCLTSDSNIRETVVKILSCGPASSGQRWMFKNDGTILNLYSGLVLDVRRSDPSLKQIILYPLHGDPNQIWLPLF

SEQ ID NO: 60 - DNA, 98 bp, synthetic construct
  misc_feature 1..98: note = Description of Artificial Sequence: Synthetic oligonucleotide
  source 1..98: mol_type = other DNA
  CDS 1..72
aagggtgacgctgaaagatgggtttctggtccaggcgcgccacttctaaataagcgaatttcttatgatttatgatttttattattaaataagttata

SEQ ID NO: 61 - DNA, 112 bp, synthetic construct
  misc_feature 1..112: note = Description of Artificial Sequence: Synthetic polynucleotide
  source 1..112: mol_type = other DNA
  CDS 1..111
gagcagaagctaatctcagaggaggacctgtttaaaccaggaggcggttctggtccaggtactgaagacgctgaagctgttgctttgggtttgggtttgtctgacttcccat

SEQ ID NO: 62 - DNA, 114 bp, synthetic construct
  misc_feature 1..114: note = Description of Artificial Sequence: Synthetic polynucleotide
  misc_difference 33..62: note = modified_base - a, c, t, g, unknown or other
  source 1..114: mol_type = other DNA
  CDS 69..113
aacgtcaaggagaaaaaactataatggttaatnnnnnnnnnnnnnnnnnnnnnnnnnnnnnntaatagacaagctacgttgaaacaagaacccgcctcctttcagaactcactt

SEQ ID NO: 63 - DNA, 7737 bp, synthetic construct
  misc_feature 1..7737: note = Description of Artificial Sequence: Synthetic polynucleotide
  source 1..7737: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccgtcttctgtcccagttaccgaatctaagggcactaccaccaaagaaacaggtgttactaccaaacaaaccacagccaacccaagtctaaccgtctccacagtcgtcccagtttcatcctctgcttcttctcattccgttgtcatcaacagtaacggtgctaacgtcgtcgttccaggtgctttaggtttggctggtgttgctatgttattcttataaacggtggtgtttgacacatccgccttcttaatgctttctttcagtattatgttatttttttgttattcgtttttcacttctaggctttttgacagactagccccgttataccaccatctttgtgggaaagcccctaaattgccctgagcagtatcgtttcatgtctagtctctttaaagatgtttcttacacttctcctatgcacatatattaattaaagtccaatgctagtagagaaggggggtaacacccctccgcgctcttttccgatttttttctaaaccgtggaatatttcggatatccttttgttgtttccgggtgtacaatatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactccaagctttgcaaagatggggtcaaaggccgagctaatcccagagccccctaaaaaaaagagaaaggtcgagctgggaactgcggcagagtacccgtatgatgtaccggactatgccggaggtatgtctagattggacaagtctaaggttatcaactctgctttggaattgttgaacgaagttggtatcgaaggtttgactactagaaagttggctcaaaagttgggtgttgaacaaccaactttgtactggcacgttaagaacaagagagctttgttggacgctttggctatcgaaatgttggacagacaccacactcacttctgtccattggaaggtgaatcttggcaagacttcttgagaaacaacgctaagtctttcagatgtgctttgttgtctcacagagacggtgctaaggttcacttgggtactagaccaactgaaaagcaatacgaaactttggaaaaccaattggctttcttgtgtcaacaaggtttctctttggaaaacgctttgtacgctttgtctgctgttggtcacttcactttgggttgtgttttggaagaccaagaacaccaagttgctaaggaagaaagagaaactccaactactgactctatgccaccattgttgagacaagctatcgaattgttcgaccaccaaggtgctgaaccagctttcttgttcggtttggaattgatcatctgtggtttggaaaagcaattgaagtgtgaatctggttctgggcaaccatctttgagatctgaatacgaatacccagttttctctcacgttcaagctggtatgttctctccagaattgagaactttcactaagggtgacgctgaaagatgggtttctggtccaggcgcgccacttctaaataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcagatctgtacaatcttgatccggagcttttctttttttgccgattaagaattaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctgcggtgtgaaataccgcacagatgcgtaaggaatatttcggatatccttttgttgtttccgggtgtacaatatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactccaagctttgcaaagatggggtcaaaggccgagctaatcccagagccccctaaaaaaaagagaaaggtcgagctgggaactgcggcagaggagcagaagctgatctcagaggaggacctgtttaaaccaggaggcggttctggtccaggtactgaagacgctgaagctgttgctttgggtttgggtttgtctgacttcccatctgctggtaaggctgttttggacgacgaagactctttcgtttggccagctgcttctttcgacatgggtgcttgttgggctggtgctggtttcgctgacccagacccagcttgtatcttcttgaacttgccatgagcccatcttttttttggacctaaattcttcatgaaaatatattacgagggcttattcagaagctttggacttcttcgccagaggtttggtcaagtctccaatcaaggttgtcggcttgtctaccttgccagaaatttacgaaaagatggaaaagggtcaaatcgttggtagatacgttgttgacacttctaaataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggccggtcgaaattcccctaccctatgaacatattccattttgtaatttcgtgtcgcgttgcgtgtaaaacatcctctcattcaagacagggttttctaaaagcaataggggtagtttaataattcttatataatcatcatatacactatttttagttcttaattctttaatacaaacttattaatgtgctctccattgatctcttaatcaggaggcgatatataccggaagcggtgtacttttcttcacctcttactcaactatgttgatgtgcaagtttaaccactcgtcgatattatctattgctataacgaaaactttattcgagttcacagtgaaaaacttcagcacatttatggaagatctaagcaaaatggagaacgccagtagatgcgaacaacaaactttatcaaatttgaaataccactgctttgataagctatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 64 - DNA, 10004 bp, synthetic construct
  misc_feature 1..10004: note = Description of Artificial Sequence: Synthetic polynucleotide
  misc_difference 1458..2224: note = modified_base - a, c, t, g, unknown or other
  misc_difference 2945..2974: note = modified_base - a, c, t, g, unknown or other
  source 1..10004: mol_type = other DNA
tgcatgcctgcaggtcgagatccgggatcgaagaaatgatggtaaatgaaataggaaatcaaggagcatgaaggcaaaagacaaatataagggtcgaacgaaaaataaagtgaaaagtgttgatatgatgtatttggctttgcggcgccgaaaaaacgagtttacgcaattgcacaatcatgctgactctgtggcggacccgcgctcttgccggcccggcgataacgctgggcgtgaggctgtgcccggcggagttttttgcgcctgcattttccaaggtttaccctgcgctaaggggcgagattggagaagcaataagaatgccggttggggttgcgatgatgacgaccacgacaactggtgtcattatttaagttgccgaaagaacctgagtgcatttgcaacatgagtatactagaagaatgagccaagacttgcgagacgcgagtttgccggtggtgcgaacaatagagcgaccatgaccttgaaggtgagacgcgcataaccgctagagtactttgaagaggaaacagcaatagggttgctaccagtataaatagacaggtacatacaacactggaaatggttgtctgtttgagtacgctttcaattcatttgggtgtgcactttattatgttacaatatggaagggaactttacacttctcctatgcacatatattaatcataagttgaattcgacaggttatcagcaacaacacagtcatatccattctcaattagctctaccacagtgtgtgaaccaatgtatccagcaccacctgtaaccaaaacaattttacctcgatcgagtttaccactccctatcagtgatagagaaaagttaaagtcgagtttaccactccctatcagtgatagagaacagtcaaagtcgagtttaccactccctatcagtgatagagaaaagttaaagtcgagtttaccactccctatcagtgatagagaaaagttaaagtcgagtttaccactccctatcagtgatagagaacagtcaaagtcgagtttaccactccctatcagtgatagagaattgtgaaagtcgagtttaccactccctatcagtgatagagaaaagtccaagtcgagctcggtaccctatggcatgcatgtggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaactataatgcaaaaatctcattcagaagaagtgattgtacctgagttcaattctagcgcaaaggaattaccaagaccattggccgaaaagtgcccgagcggtgctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntaatagcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgatacggattagaagccgccgagcgggcgacagccctccgacggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaactataatggttaatnnnnnnnnnnnnnnnnnnnnnnnnnnnnnntaatagacaagctacgttgaaacaagaacccgcctcctttcagaactcacttacggtacattagcgatgactacgacttcattactctttttttcagaaaattttaatcaatattcatttattctacgaacagtctccttcaccttagtttctttctctgctcctttgaaactattattgtatttggtacattttagagaaaataaaacatatatagaacaatgagaaggtacgtaatttcttagctaattatttgtaatcaattaagcgtcttcctttagcaaagcgtctctcttttcagcaactctttgagctcttctgtcacgagcagctctgttcttcaatcttctagcttcagcttcttcgttcaaagccttttcacgttgagcatcagccttagcttgaataatgtgttcaaccaaagctctcttgtgcttgaaggcgttacccttggattccttgtacaaaacgtggtacaaatgcttgtcaatcttaccagcgtcacggtacttggccaataatcttctcaagacacgtaatcttctgatccagacgacttgggatggtaaacgagcttctctagtaccctttctcttaccgtaaccactgtgacgaccttctctcttggattgagcatgggctctagttctagatttagagtggacagtaacggccttcttgacgatggttccgttcttgaccaattttctaatggcatttctagagttggcttgggcaatttcagaagtttcgtttgggtctaaccaaacctttctcttaccaacaccgacaacagaagcggcaagtctcttttgagtacgcaagttagccctgtgaaaaaaagtttttgcagatttatttgcatattgatgttagtaaagttgcttcatttttaaaatcctgaaacctaacagtaaagagcatattcgcaaaggttaatgaattactttatctatcaatcgaattaacgcttgcaggaacagacacgtaccattgctgcgataattctatagtttgtaataaacgcggcaattcgtacaagcttgaaatttatctgaggttcttctatggatgttgctaccaactatgcgaccaccggatgctgtatcctcaatttttttccttatctatttctctccaaaggatgacattcataacatatttaaagataaatcttgtgaaaggttcaaaatttagtatcactgttaaacatacattttcctctaatttattggtgactttttattcgatttggtgaaaagatctatcaagtagcactagcgtataaatgtactattagtatcccgatgtagatacagtaagctttggacttcttcgccagaggtttggtcaagtctccaatcaaggttgtcggcttgtctaccttgccagaaatttacgaaaagatggaaaagggtcaaatcgttggtagatacgttgttgacacttctaaataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggccggtcgaaattcccctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggttgaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtaggaaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactacctgtaccccatttaggaccacccacagcacctaacaaaacggcatcagccttcttggaggcttccagcgcctcatctggaagtgggacacctgtagcttcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcgctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttgggggatctcgactctagctagaggatcaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgataacttcgtataatgtatgctatacgaagttattaggtctgaagaggagtttacgtccagccaagctagcttggctgcaggtcgagcggccgcgatccggaacccttaatataacttcgtataatgtatgctatacgaagttatcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttgct

SEQ ID NO: 65 - DNA, 7781 bp, synthetic construct
  misc_feature 1..7781: note = Description of Artificial Sequence: Synthetic polynucleotide
  source 1..7781: mol_type = other DNA
cggtgcgggcctcttcgctattacgccagatccttttgttgtttccgggtgtacaatatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactccaagctttgcaaagatggggtcaaaggccgagctaatcccagagccccctaaaaaaaagagaaaggtcgagctgggaactgcggcagagtacccgtatgatgtaccggactatgccggaggtatgtctagattggacaagtctaaggttatcaactctgctttggaattgttgaacgaagttggtatcgaaggtttgactactagaaagttggctcaaaagttgggtgttgaacaaccaactttgtactggcacgttaagaacaagagagctttgttggacgctttggctatcgaaatgttggacagacaccacactcacttctgtccattggaaggtgaatcttggcaagacttcttgagaaacaacgctaagtctttcagatgtgctttgttgtctcacagagacggtgctaaggttcacttgggtactagaccaactgaaaagcaatacgaaactttggaaaaccaattggctttcttgtgtcaacaaggtttctctttggaaaacgctttgtacgctttgtctgctgttggtcacttcactttgggttgtgttttggaagaccaagaacaccaagttgctaaggaagaaagagaaactccaactactgactctatgccaccattgttgagacaagctatcgaattgttcgaccaccaaggtgctgaaccagctttcttgttcggtttggaattgatcatctgtggtttggaaaagcaattgaagtgtgaatctggttctgggcaaccatctttgagatctgaatacgaatacccagttttctctcacgttcaagctggtatgttctctccagaattgagaactttcactaagggtgacgctgaaagatgggtttctggtccaggtactgaagacgctgaagctgttgctttgggtttgggtttgtctgacttcccatctgctggtaaggctgttttggacgacgaagactctttcgtttggccagctgcttctttcgacatgggtgcttgttgggctggtgctggtttcgctgacccagacccagcttgtatcttcttgaacttgccatgagcccatcttttttttggacctaaattcttcatgaaaatatattacgagggcttattcagaagctttggacttcttcgcttgcagccaagctaattccgggcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaacctgaggctatggcagggcctgccgccccgacgttggctgcgagccctgggccttcacccgaacttggggggtggggtggggaaaaggaagaaacgcgggcgtattggccccaatggggtctcggtggggtatcgacagagtgccagccctgggaccgaaccccgcgtttatgaacaaacgacccaacaccgtgcgttttattctgtctttttattgccgtcatagcgcgggttccttccggtattgtctccttccgtgtttcagttagcctccccctagggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgagtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatagatcaacgacattactatatatataatataggaagcatttaatagaacagcatcgtaatatatgtgtactttgcagttatgacgccagatggcagtagtggaagatattctttattgaaaaatagcttgtcaccttacgtacaatcttgatccggagcttttctttttttgccgattaagaattaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgccgatctatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgat

SEQ ID NO: 66 - protein, 20 aa, synthetic construct
  REGION 1..20: note = Description of Artificial Sequence: Synthetic peptide
RRRRRRRKRLGVRLRVSRML

SEQ ID NO: 67 - protein, 27 aa, synthetic construct
  REGION 1..27: note = Description of Artificial Sequence: Synthetic peptide
RQKIWFQNRRMKWKKRLGVRLRVSRML

SEQ ID NO: 68 - protein, 15 aa, synthetic construct
  REGION 1..15: note = Description of Artificial Sequence: Synthetic peptide
  SITE 2: note = MOD_RES - D-2-(7'-octenyl)alanine
  SITE 2..9: note = MISC_FEATURE - Crosslink between residues
  SITE 9: note = MOD_RES - 2-(4'-pentenyl)alanine
AAKRLGVRARVSRML

SEQ ID NO: 69 - protein, 15 aa, synthetic construct
  REGION 1..15: note = Description of Artificial Sequence: Synthetic peptide
LMRSVRLRVGLRKAG

SEQ ID NO: 70-71 - skipped (2 entries)

SEQ ID NO: 72 - protein, 6 aa, synthetic construct
  REGION 1..6: note = Description of Artificial Sequence: Synthetic peptide
  VARIANT 5: note = MOD_RES - Met or Val
  VARIANT 6: note = MOD_RES - Leu, Ile, Val, Phe, Met, Ala, Trp or Tyr
RLGVXX

SEQ ID NO: 73 - protein, 8 aa, synthetic construct
  REGION 1..8: note = Description of Artificial Sequence: Synthetic peptide
  VARIANT 1..2: note = MOD_RES - Thr, Lys, Arg or Gln
  VARIANT 1..2: note = MISC_FEATURE - This region may encompass 1-2 residues
  VARIANT 7: note = MOD_RES - Met or Val
  VARIANT 8: note = MOD_RES - Leu, Ile, Val, Phe, Met, Ala, Trp or Tyr
XXRLGVXX

SEQ ID NO: 74-81 - skipped (8 entries)

SEQ ID NO: 82 - protein, 339 aa, Homo sapiens
MAMQMQLEANADTSVEEESFGPQPISRLEQCGINANDVKKLEEAGFHTVEAVAYAPKKELINIKGISEAKADKILAEAAKLVPMGFTTATEFHQRRSEIIQITTGSKELDKLLQGGIETGSITEMFGEFRTGKTQICHTLAVTCQLPIDRGGGEGKAMYIDTEGTFRPERLLAVAERYGLSGSDVLDNVAYARAFNTDHQTQLLYQASAMMVESRYALLIVDSATALYRTDYSGRGELSARQMHLARFLRMLLRLADEFGVAVVITNQVVAQVDGAAMFAADPKKPIGGNIIAHASTTRLYLRKGRGETRICKIYDSPCLPEAEAMFAINADGVGDAKD

SEQ ID NO: 83 - protein, 730 aa, unidentified
  source 1..730: note = Amanita bisporigera
MPPTPWAPHSYPPTRRSDHVDVYQSASRGEVPVPDPYQWLEENSNEVDEWTTAQTAFTQGYLDKNADRQKLEEKFRASKDYVKFSAPTLLDSGHWYWFYNSGVQSQAVLYRSKKPVLPDFQRGTRKVGEVYFDPNVLSADGTAIMGTCRFSPSGEYFAYAVSHLGVDYFTIYVRPTSSSLSQAPEAEGGDGRLSDGVKWCKFTTITWTKDSKGFLYQRYPARESLVAKDRDKDAMVCYHRVGTTQLEDIIVQQDKENPDWTYGTDASEDGKYIYLVVYKDASKQNLLWVAEFDKDGVKPEIPWRKVINEFGADYHVITNHGSLIYVKTNVNAPQYKVVTIDLSTGEPEIRDFIPEQKDAKLTQVKCVNKGYFVAIYKRNVKDEIYLYSKAGDQLSRLASDFIGVASITNREKQPHSFLTFSGFNTPGTISRYDFTAPDTQRLSILRTTKLNGLNADDFESTQVWYKSKDGTKVPMFIVRHKSTKFDGTAPAIQNGYGGFAITADPFFSPIMLTFMQTYGAILAVPNIRGGGEFGGEWHKAGRRETKGNTFDDFIAAAQFLVKNKYAAPGKVAITGASNGGFLVCGSVVRAPEGTFGAAVSEGGVADLLKFNKFTGGMAWTSEYGNPFIKEDFDFVQALSPVHNVPKDRVLPATLLMTNAGDDRVVPMHSLKFVANLQYNVPQNPHPLLIRVDKSWLGHGFGKTTDKHTKDAADKWSFVAQSLGLEWKTVD

SEQ ID NO: 84 - protein, 730 aa, unidentified
  source 1..730: note = Galerina marginata
MSSVTWAPGNYPSTRRSDHVDTYQSASKGEVPVPDPYQWLEESTDEVDKWTTAQADLAQSYLDQNADIQKLAEKFRASRNYAKFSAPTLLDDGHWYWFYNRGLQSQSVLYRSNEPALPDFSNGDDNVGDVFFDPNVLATDGSAGMVLCKFSPDGKFFAYAVSHLGGDYSTIYIRSTSSPLSQASAVQGTDGRLSDEVKWFKFSTIIWTKDSKGFLYQRYPARERHEGTRSDRNAMMCYHKVGTTQEEDIIVYQDNEHPEWIYGADTSEDGKYLYLYQFKDTSKKNLLWVAELNEDGVKSGIQWRKVVNEYVADYNVITNHGSLVYIKTNLNAPQYKVITIDLSKDEPEIRDFIPEEKDAKLAQVNCANEEYFVAIYKRNVKDEIYLYSKAGVQLTRLAPDFVGAASIANRQKQTHFFLTLSGFNTPGTIARYDFTAPETQRFSILRTTKVNELDPDDFESTQVWYESKDGTKIPMFIVRHKSTKFDGTAAAIQYGYGGFATSADPFFSPIILTFLQTYGAIFAVPSIRGGGEFGEEWHKGGRRETKVNTFDDFIAAAQFLVKNKYAAPGKVAINGASNGGLLVMGSIVRAPEGTFGAAVPEGGVADLLKFHKFTGGQAWISEYGNPSIPEEFDYIYPLSPVHNVRTDKVMPATLITVNIGDGRVVPMHSFKFIATLQHNVPQNPHPLLIKIDKSWLGHGMGKPTDKNVKDAADKWGFIARALGLELKTVE

SEQ ID NO: 85 - protein, 29 aa, synthetic construct
  REGION 1..29: note = Description of Artificial Sequence: Synthetic peptide
  SITE 1: note = N-term Acetylation
  SITE 1: note = MOD_RES - Fluorescein amidite label linked via a 6 carbon chain to the epsilon amino group of lysine (Lys-(C6-5FAM))
  SITE 29: note = C-term amidation
KQSLRLGLSRLARVKRLHPGARRRRRRRR

SEQ ID NO: 86 - protein, 29 aa, synthetic construct
  REGION 1..29: note = Description of Artificial Sequence: Synthetic peptide
  SITE 1: note = N-term Acetylation
  SITE 1: note = MOD_RES - Fluorescein amidite label linked via a 6 carbon chain to the epsilon amino group of lysine (Lys-(C6-5FAM))
  SITE 29: note = C-term amidation
KQSLRLGQSRLARVKRLHPGARRRRRRRR

SEQ ID NO: 87 - protein, 21 aa, synthetic construct
  REGION 1..21: note = Description of Artificial Sequence: Synthetic peptide
  SITE 1: note = N-term Acetylation
  SITE 1: note = MOD_RES - Fluorescein amidite label linked via a 6 carbon chain to the epsilon amino group of lysine (Lys-(C6-5FAM))
  SITE 4: note = MOD_RES - N-methyl Leucine (NMe-Leu)
  SITE 11: note = MOD_RES - N-methyl Leucine (NMe-Leu)
  SITE 14: note = MOD_RES - N-methyl Valine (NMe-Val)
  SITE 21: note = C-term amidation
KQSLRLGLSRLARVKRLHPGC

SEQ ID NO: 88 - protein, 9 aa, synthetic construct
  REGION 1..9: note = Description of Artificial Sequence: Synthetic peptide
  SITE 1: note = N-term Acetylation
  SITE 9: note = C-term amidation
CRRRRRRRR

SEQ ID NO: 89 - protein, 21 aa, synthetic construct
  REGION 1..21: note = Description of Artificial Sequence: Synthetic peptide
  SITE 1: note = N-term Acetylation
  SITE 1: note = MOD_RES - Fluorescein amidite label linked via a 6 carbon chain to the epsilon amino group of lysine (Lys-(C6-5FAM))
  SITE 21: note = C-term amidation
KRRRRRRRKRLGVRLRVSRML

SEQ ID NO: 90 - protein, 21 aa, synthetic construct
  REGION 1..21: note = Description of Artificial Sequence: Synthetic peptide
  SITE 1: note = N-term Acetylation
  SITE 1: note = MOD_RES - Fluorescein amidite label linked via a 6 carbon chain to the epsilon amino group of lysine (Lys-(C6-5FAM))
  SITE 21: note = C-term amidation
KRRRRRRRKRLGQRLRQSRML

SEQ ID NO: 91 - protein, 16 aa, synthetic construct
  REGION 1..16: note = Description of Artificial Sequence: Synthetic peptide
  SITE 1: note = N-term Acetylation
  SITE 1: note = MOD_RES - Fluorescein amidite label linked via a 6 carbon chain to the epsilon amino group of lysine (Lys-(C6-5FAM))
  SITE 16: note = C-term amidation
KRRRRRRRRLGMSRRF

SEQ ID NO: 92 - protein, 21 aa, synthetic construct
  REGION 1..21: note = Description of Artificial Sequence: Synthetic peptide
  SITE 1: note = N-term Acetylation
  SITE 1: note = MOD_RES - Fluorescein amidite label linked via a 6 carbon chain to the epsilon amino group of lysine (Lys-(C6-5FAM))
  SITE 21: note = C-term amidation
KRRRRRRRKRLGLRLGVSRRV

SEQ ID NO: 93 - protein, 26 aa, synthetic construct
  REGION 1..26: note = Description of Artificial Sequence: Synthetic peptide
  SITE 1: note = N-term Acetylation
  SITE 1: note = MOD_RES - Fluorescein amidite label linked via a 6 carbon chain to the epsilon amino group of lysine (Lys-(C6-5FAM))
  SITE 26: note = C-term amidation
KRRRRRRRSLRLGLSRLARVKRLHPG

SEQ ID NO: 94 - protein, 20 aa, synthetic construct
  REGION 1..20: note = Description of Artificial Sequence: Synthetic peptide
  SITE 1: note = N-term Acetylation
  SITE 20: note = C-term amidation
RRRRRRRKRLGVRLRVSRML

SEQ ID NO: 95 - protein, 27 aa, synthetic construct
  REGION 1..27: note = Description of Artificial Sequence: Synthetic peptide
  SITE 1: note = N-term Acetylation
  SITE 27: note = C-term amidation
RQKIWFQNRRMKWKKRLGVRLRVSRML

SEQ ID NO: 96 - protein, 15 aa, synthetic construct
  REGION 1..15: note = Description of Artificial Sequence: Synthetic peptide
  SITE 1: note = N-term Acetylation
  SITE 2: note = MOD_RES - D-2-(7'-octenyl)alanine
  SITE 2..9: note = MISC_FEATURE - Crosslink between residues
  SITE 9: note = MOD_RES - 2-(4'-pentenyl)alanine
  SITE 15: note = C-term amidation
AAKRLGVRARVSRML

SEQ ID NO: 97 - protein, 24 aa, synthetic construct
  REGION 1..24: note = Description of Artificial Sequence: Synthetic peptide
KGDAERWVSGPGAPLLNKRISYDL

SEQ ID NO: 98 - protein, 37 aa, synthetic construct
  REGION 1..37: note = Description of Artificial Sequence: Synthetic polypeptide
EQKLISEEDLFKPGGGSGPGTEDAEAVALGLGLSDFP

SEQ ID NO: 99 - protein, 15 aa, synthetic construct
  REGION 1..15: note = Description of Artificial Sequence: Synthetic peptide
TSYVETRTRLLSELT

SEQ ID NO: 100 - protein, 29 aa, synthetic construct
  REGION 1..29: note = Description of Artificial Sequence: Synthetic polypeptide
  SITE 1: note = N-term Acetylation
  SITE 1: note = MOD_RES - Fluorescein amidite label linked via a 6 carbon chain to the epsilon amino group of lysine (Lys-(C6-5FAM))
  SITE 4: note = MOD_RES - N-methyl Leucine (NMe-Leu)
  SITE 11: note = MOD_RES - N-methyl Leucine (NMe-Leu)
  SITE 14: note = MOD_RES - N-methyl Valine (NMe-Val)
  SITE 29: note = C-term amidation
KQSLRLGLSRLARVKRLHPGARRRRRRRR

SEQ ID NO: 101 - protein, 87 aa, Homo sapiens
YIDTEGTFRPERLLAVAERYGLSGSDVLDNVAYARAFNTDHQTQLLYQASAMMVESRYALLIVDSATALYRTDYSGRGELSARQMHL

SEQ ID NO: 102 - protein, 87 aa, Gallus gallus
YIDTEGTFRPERLLAVAERYGLSGSDVLDNVAYARGFNTDHQTQLLYQASAMMAESRYALLIVDSATALYRTDYSGRGELSARQMHL

SEQ ID NO: 103 - protein, 87 aa, Danio rerio
YIDTEGTFRPERLLAVAERYGLVGSDVLDNVAYARAFNTDHQTQLLYQASAMMTESRYALLIVDSATALYRTDYSGRGELSARQGHL

SEQ ID NO: 104 - protein, 87 aa, Xenopus laevis
YIDTEGTFRPERLLAVAERYGLSGSDVLDNVAYARAFNTDHQTQLLYQASAMMAESRYALLIVDSATALYRTDYSGRGELSARQMHL

SEQ ID NO: 105 - protein, 87 aa, Ustilago maydis
YIDTENTFRPTRLLAVAERFGLNGEEVLDNVAYARAYNADHQLQLLMQASAMMAESRFSLLIVDSLTSLYRTDFSGRGELSARQMHL

SEQ ID NO: 106 - protein, 87 aa, unidentified
  source 1..87: note = Schizosaccharomyces pombe
YIDTEGTFRPVRLLAVADRYGLNGEEVLDNVAYARAYNADHQLELLQQAANMMSESRFSLLVVDSCTALYRTDFSGRGELSARQMHL

SEQ ID NO: 107 - protein, 87 aa, unidentified
  source 1..87: note = Schizosaccharomyces japonicus
YIDTEGTFRPVRLLAVAERYGLNGEEVLDNVAYARAYNADHQLELLQQAANMMAESRFSLLVVDSCTALYRTDFSGRGELSARQMHL

SEQ ID NO: 108 - protein, 87 aa, Pichia sp.
  source 1..87: note = Pichia membranifaciens
FIDTESTFRPIRIVPIARRFGLDEHEAMENIAYARAYNADHQLQLLNQAAQMMAQSRFSLLVVDSIMALYRTDYSGRGELSARQMHV

SEQ ID NO: 109 - protein, 87 aa, Pichia sp.
  source 1..87: note = Pichia kudriavzevii
FIDTESTFRPVRIVPIARRFGLDEREAMENIAYARAYNADHQLQLLNQAAQMMAQSRFSLLIVDSIMALYRTDYSGRGELSARQMHV

SEQ ID NO: 110 - protein, 87 aa, unidentified
  source 1..87: note = Arthrobotrys oligospora
YIDTEGTFRPVRLLAVAQRYGLNGEEVLDNVAYARAYNSDHQLQLLNMAAQMMTETRFSLLIVDSATSLYRTDFSGRGDLSARQMHL

SEQ ID NO: 111 - protein, 87 aa, unidentified
  source 1..87: note = Dactylellina haptotyla
YIDTEGTFRPVRLVSIAQRYGLNPEDCLDNVAYARAYNAEHQFQLLNHAAQMMSESRFSCLIVDSIMSLYRTDYSGRAELSARQTHV

SEQ ID NO: 112 - protein, 87 aa, unidentified
  source 1..87: note = Millerozyma farinose
YIDTEGTFRPVRLVSIAQRYGLNPDDCLDNVAYARAYNADHQFQLLNLAAQMMAESRFSLLIVDSIMSLYRTDYSGRGELSARQSHV

SEQ ID NO: 113 - protein, 87 aa, Debaryomyces hansenii; identical to SEQ ID NO: 111

SEQ ID NO: 114 - protein, 87 aa, unidentified
  source 1..87: note = Ogataea parapolymorpha
YIDTEGTFRPIRLVAIARRFGLDENETLDNVAYARAYNADHQLQLLHQAASMMTESRFSLLIVDSIMALYRTDYSGRGELSARQMHV

SEQ ID NO: 115 - protein, 87 aa, unidentified
  source 1..87: note = Komagataella phaffii
YIDTEGTFRPIRLVSIAKRYGLNEDDTLDNVAYARAYNADHQLQLLNQAAAMMSESRFSLLIVDSIMALYRTDFSGRGELSARQMHV

SEQ ID NO: 116 - protein, 87 aa, unidentified
  source 1..87: note = Aspergillus clavatus
YIDTEGTFRPVRLLAVAQRYGLVGEEVLDNVAYARAYNSDHQLQLLNQASQMMCETRFSLLVVDSATSLYRTDFNGRGELSSRQTHL

SEQ ID NO: 117 - protein, 87 aa, unidentified
  source 1..87: note = Pneumocystis jirovecii
YIDTEGTFRPTRLLAVADRYGLNGEEVLDNVAYARAYNADHQLQLLGQAANMMAESRFSLLIVDSATSLYRTDFAGRGELSARQMHL

SEQ ID NO: 118 - protein, 87 aa, unidentified
  source 1..87: note = Yarrowia lipolytica
YIDTEGTFRPERLHQIARRYGLNGDEVLDNVAYARAHNSDHQMQLLQMAANMMTKSRFSCLIVDSIMALYRTDYAGRGELSARQTHV

SEQ ID NO: 119 - protein, 87 aa, Candida albicans
YIDTEGTFRPNRLVSIAQRYGLNPNDCLDNVAYARAYNAEHQLNLLNIAAEMMAESRFSLLIVDSIMSLYRTDYAGRGELSARQTHV

SEQ ID NO: 120 - protein, 87 aa, unidentified
  source 1..87: note = Wickerhamomyces ciferrii
YIDTEGTFRPVRLVSIARRYGLNEDDALDNVAYARAYNADHQLQLLNQAAAMMSESRFSLLIVDSIMALYRTDFAGRGELSARQMHV

SEQ ID NO: 121 - protein, 87 aa, unidentified
  source 1..87: note = Cyberlindnera fabianii
YIDTEGTFRPVRLVSIARRYGLNEDDALDNVAYARAYNADHQLSLLNQAAAMMSESRFSLLIVDSIMALYRTDFAGRGELSARQMHV

SEQ ID NO: 122 - protein, 87 aa, Candida sp.
  source 1..87: note = Candida dubliniensis
YIDTEGTFRPNRLVSIAQRYGLNPNDCLDNVAYARAYNAEHQLNLLNIASQMMAESRFSLLIVDSIMSLYRTDYAGRGELSARQTHV

SEQ ID NO: 123 - protein, 87 aa, unidentified
  source 1..87: note = Lodderomyces elongisporus
YIDTEGTFRPNRLVSIAERYGLNANDCLDNVAYARAYNAEHQLNLLNLAAEMMAESRFSLLIVDSIMSLYRTDYAGRGELSARQTSV

SEQ ID NO: 124 - protein, 87 aa, Candida sp.
  source 1..87: note = Candida maltosa
YIDTEGTFRPNRLISIAERYGLNPNDCLDNVAYARAYNAEHQLNLLNVAAQMMAESRFSLLIVDSIMSLYRTDYAGRGELSARQTHV

SEQ ID NO: 125 - protein, 87 aa, Candida tropicalis
YIDTEGTFRPNRLISIAERYGLNANDCLDNVAYARAYNAEHQLNLLNIAAQMMAESRFSLLIVDSIMSLYRTDYAGRGELSARQTHV

SEQ ID NO: 126 - protein, 87 aa, unidentified
  source 1..87: note = Zygosaccharomyces bailii
YIDTEGTFRPVRLVSIAQRFGLDPDDALNNVAYARAYNADHQIRLLDAAAQMMSESRFSLVVVDSVMALYRTDFSGRGELSARQMHL

SEQ ID NO: 127 - protein, 87 aa, unidentified
  source 1..87: note = Lachancea mirantina
YIDTEGTFRPIRLVSIAQRFGLDPDDALNNVAYARAYNADHQLRLLDAAAQMMSESRFSLIVVDSIMALYRTDFSGRGELSARQMHL

SEQ ID NO: 128 - protein, 87 aa, Candida sp.
  source 1..87: note = Candida glabrata
YIDTEGTFRPVRLVSIAQRFGLDPDDALNNVAYARAYNADHQMRLLDAAAQMMSESRFSLIVVDSVMALYRTDFSGRGELSARQMHL

SEQ ID NO: 129 - protein, 87 aa, unidentified
  source 1..87: note = Kazachstania saulgeensis
YIDTEGTFRPIRLVSIAQRFGLDPDDALNNVAYARAYNADHQLKLLDAAAQMMSESRFSLIVVDSVMALYRTDFSGRGELSARQMHL

SEQ ID NO: 130 - protein, 87 aa, unidentified
  source 1..87: note = Naumovozyma dairenensis
YIDTEGTFRPVRLVSIAQRFGLDPDDALNNVAYARAYNADHQLKLLDAASQMMSESRFSLIIVDSVMALYRTDFSGRGELSARQMHL

SEQ ID NO: 131 - protein, 87 aa, Kluyveromyces sp.
  source 1..87: note = Kluyveromyces lactis
YIDTEGTFRPIRLVSIAQRFGLDPDDALNNVAYARAYNADHQLKLLDAAAQMMSESRFSLIIVDSIMALYRTDFSGRGELSARQMHL

SEQ ID NO: 132 - protein, 87 aa, Kluyveromyces sp.; identical to SEQ ID NO: 131
  source 1..87: note = Kluyveromyces dobzhanskii

SEQ ID NO: 133 - protein, 87 aa, unidentified; identical to SEQ ID NO: 126
  source 1..87: note = Zygosaccharomyces rouxii

SEQ ID NO: 134 - protein, 87 aa, unidentified
  source 1..87: note = Naumovozyma castelli
YIDTEGTFRPVRLVSIAQRFGLDPDDALNNVAYARAYNADHQLRLLDAAAQMMSESRFSLIIVDSVMALYRTDFSGRGELSARQMHL

SEQ ID NO: 135 - protein, 87 aa, unidentified
  source 1..87: note = Torulaspora delbrueckii
YIDTEGTFRPVRLVSIAQRFGLDPDDALNNVAYARAYNADHQIRLLDAAAQMMSESRFSLIVVDSVMALYRTDFSGRGELSARQMHL

SEQ ID NO: 136 - protein, 87 aa, unidentified
  source 1..87: note = Lachancea dasiensis
YIDTEGTFRPIRLVSIAQRFGLDPDDALNNVAYARAYNADHQLRLLDAAAQMMSESRFSLIVVDSIMALYRTDFAGRGELSARQMHL

SEQ ID NO: 137 - protein, 87 aa, unidentified
  source 1..87: note = Kazachstania africana
YIDTEGTFRPIRLVSIAQRFGLDPDDALNNVAYARAYNADHQLRLLDAAAQMMSESRFSLIIVDSVMALYRTDFSGRGELSARQMHL

SEQ ID NO: 138 - protein, 87 aa, Eremothecium sp.; identical to SEQ ID NO: 127
  source 1..87: note = Eremothecium sinecaudum

SEQ ID NO: 139 - protein, 87 aa, unidentified; identical to SEQ ID NO: 127
  source 1..87: note = Ashbya aceri

SEQ ID NO: 140 - protein, 87 aa, unidentified; identical to SEQ ID NO: 135
  source 1..87: note = Vanderwaltozyma polyspora

SEQ ID NO: 141 - protein, 87 aa, Eremothecium sp.; identical to SEQ ID NO: 127
  source 1..87: note = Eremothecium gossypii

SEQ ID NO: 142 - protein, 87 aa, unidentified
  source 1..87: note = Lachancea nothofagi
YIDTEGTFRPIRLVSIAQRFGLDPDDALNNVAYARAYNADHQLRLLDAAAQMMSESRFSVIVVDSIMALYRTDFSGRGELSARQMHL

SEQ ID NO: 143 - protein, 87 aa, Eremothecium sp.; identical to SEQ ID NO: 127
  source 1..87: note = Eremothecium cymbalariae

SEQ ID NO: 144 - protein, 87 aa, Saccharomyces cerevisiae
YIDTEGTFRPVRLVSIAQRFGLDPDDALNNVAYARAYNADHQLRLLDAAAQMMSESRFSLIVVDSVMALYRTDFSGRGELSARQMHL

(19) United States
(12) Patent Application Publication
(10) Pub. No.: US 2025/0177474 A1
(43) Pub. Date: Jun. 5, 2025

(54) PROTEIN INTERFACES

(71) Applicant: SyntheX, Inc., San Francisco, CA (US)

(72) Inventors: Charly Chahwan, San Francisco, CA (US); Maria Soloveychik, San Francisco, CA (US)

(21) Appl. No.: 18/975,990

(22) Filed: Dec. 10, 2024

Related U.S. Application Data
(62) Division of application No. 17/171,841, filed on Feb. 9, 2021, now Pat. No. 12,214,008.
Application No. 17/171,841 is a continuation of application No. 16/215,432, filed on Dec. 10, 2018, now abandoned.
Application No. 16/215,432 is a continuation of application No. 15/683,586, filed on Aug. 22, 2017, now Pat. No. 10,188,691.
Application No. 15/683,586 is a continuation of application No. PCT/US17/34870, filed on May 26, 2017.
(60) Provisional application No. 62/384,226, filed on Sep. 7, 2016.
(60) Provisional application No. 62/342,840, filed on May 27, 2016.

(51) Int. Cl. A61K 38/02; A61K 38/00; A61K 39/00; A61K 45/06; C07K 5/11; C07K 7/06; C07K 7/08; C07K 14/00; C07K 16/30

(52) U.S. Cl. CPC A61K 38/02; A61K 45/06; C07K 5/1019; C07K 7/06; C07K 7/08; C07K 14/00; C07K 16/30; A61K 38/00; A61K 2039/585; Y02A 50/30

(57) ABSTRACT

The present disclosure provides methods to treat conditions, including cancer, using compounds that can target resistant cancer cells. The compounds can be used to sensitize resistant cancer cells or decrease the proliferation of cells. The compounds can target proteins in the DNA damage repair pathway leading to a decrease in DNA damage repair in target cells.

### CROSS-REFERENCE

This application is a divisional of U.S. patent application Ser. No. 17/171,841, filed Feb. 9, 2021, which is a continuation of U.S. patent application Ser. No. 16/215,432, filed Dec. 10, 2018, which is a continuation of U.S. patent application Ser. No. 15/683,586, filed Aug. 22, 2017, now U.S. Pat. No. 10,188,691, which is a continuation of PCT Application No. PCT/US17/34870, filed on May 26, 2017, which claims the benefit of U.S. Provisional Appl. No. 62/342,840, filed on May 27, 2016, and Provisional Application No. 62/384,226, filed on Sep. 7, 2016. All of these applications are hereby incorporated by reference in their entireties.

### SEQUENCE LISTING

The instant application contains a Sequence Listing which has been submitted electronically in XML format and is hereby incorporated by reference in its entirety. Said XML copy, created on Dec. 10, 2024, is named 50607-701.401.xml and is 220,854 bytes in size.

### BACKGROUND

Cancer, an uncontrolled proliferation of cells, is a multifactorial disease characterized by tumor formation, growth, and in some instances, metastasis. Current cancer therapies include chemotherapy and targeted therapies, which attempt to destroy cancer cells via apoptosis, necrosis, or proliferative inhibition. Deoxyribonucleic acid (DNA) repair pathways are frequently overexpressed in cancer cells, and can be essential to the proliferation of chemotherapy-resistant cancers. Thus, compounds that can attenuate aberrant DNA damage repair pathway signaling could be beneficial to cancer patients. However, such signaling pathways in DNA repair and cancer frequently involve protein-protein interactions as critical regulatory steps, making the traditional enzyme active-site inhibitor-based drug development scheme challenging. Accordingly, there is need for development of methods and compositions that target protein-protein interactions in cancer and DNA repair.

### SUMMARY

The present disclosure is directed to methods, compositions, and techniques for the identification and production of protein-protein interaction inhibitors as well as methods, compositions, and techniques for using protein-protein interaction inhibitors.

In one aspect, the invention relates to a non-naturally occurring compound which interacts with RAD51AP1's binding site on human RAD51. In one embodiment the compound interacts with at least one of residues 202, 205, and 206 of human RAD51. In one embodiment, the compound further interacts with residue 187 of human RAD51. In another embodiment, the compound has a binding constant to RAD51 with a Kd value of 10^{÷}M or less. In another embodiment, the compound comprises a polypeptide. In one embodiment, the polypeptide comprises non-naturally occurring amino acids. In another embodiment, the polypeptide comprises both (L)- and (D)-amino acids. In another embodiment, the polypeptide comprises at least one (D)-amino acid. In another embodiment the compound comprises an amino acid sequence according to formula I, or an invert thereof, wherein any one or more of the amino acids are optionally non-natural amino acids or (D)-amino acids. In another embodiment, the compound comprises an amino acid sequence according to any one of formulas II-IV, or an invert thereof, wherein any one or more of the amino acids are optionally non-natural amino acids or (D)-amino acids. In another embodiment, the compound comprises an amino acid sequence according to SEQ ID NO.: 1, SEQ ID NO.: 5, SEQ ID NO.: 10, SEQ ID NO.: 66, or SEQ ID NO.: 67, wherein any one or more of the amino acids are optionally non-natural amino acids or (D)-amino acids. In another embodiment, the compound is compound 1, compound 5, compound 10, compound 13, or compound 14. In another embodiment, the polypeptide is not an antibody. In another embodiment, the polypeptide consists of fewer than 30 amino acid residues. In another embodiment, the polypeptide consists of 60 or fewer amino acid residues. In another embodiment, the polypeptide consists of fewer than 30 amino acid residues. In another embodiment, the polypeptide consists of fewer than 20, fewer than 15, or fewer than 10 amino acid residues. In another embodiment, the polypeptide further comprises a cell-penetrating peptide sequence. In another embodiment, the compound displays at least one of the following characteristics upon binding to RAD51 in a cell: (a) inhibition of assembly of RAD51 monomers on DNA; (b) inhibition of cellular homologous recombination; (c) lack of inhibition of RAD51 ATPase activity; or (d) lowering of RAD51 affinity for Ca2+.

In another aspect, the invention relates to a method of inducing cell death, wherein the method comprises contacting the cell with a polypeptide that binds to a eukaryotic recombinase in a cell, wherein the binding of the polypeptide to the eukaryotic recombinase in inhibits binding of the eukaryotic recombinase to a protein in the cell, wherein the cell exhibits an increase in intracellular free calcium concentration upon binding of the polypeptide to the eukaryotic recombinase. In one embodiment, the polypeptide comprises an amino acid sequence according to formula I or an invert thereof. In another embodiment, the eukaryotic recombinase is RAD51. In another embodiment, the protein is BRCA2. In another embodiment, the protein is RAD51AP1. In another embodiment the cell death is apoptotic cell death. In another embodiment, the binding of the polypeptide to a eukaryotic recombinase in the cell sensitizes the cell to a chemotherapeutic. In another embodiment, the method further comprises administering a chemotherapeutic agent. In another embodiment, the chemotherapeutic agent is PD-L1, an anti-PD1 agent, or a PARP inhibitor. In another embodiment, the eukaryotic cell is a cancer cell. In another embodiment, the cell is a human cell. In another embodiment, the polypeptide induces death of the cell at a rate at least 10-fold greater than in the absence of the polypeptide. In another embodiment, the polypeptide induces death of a cancer cell at a rate greater than that of a non-cancerous cell. In another embodiment, the polypeptide induces death of a cancer cell at a rate at least 10-fold greater than a non-cancerous cell. In another embodiment, the polypeptide induces death of a cancer cell dependent on elevated RAD51 activity at a rate greater than that of a non-cancerous cell.

In a further aspect, the invention relates to a method of treating a condition associated with aberrant RAD51 activity, comprising administering to a subject in need thereof a therapeutically effective amount of a polypeptide which interacts with RAD51AP1's binding site on human RAD51. In one embodiment the compound interacts with at least one of residues 202, 205, and 206 of human RAD51. In one embodiment, the compound further interacts with residue 187 of human RAD51. In another embodiment, the compound has a binding constant to RAD51 with a Kd value of 10⁻⁴ M or less. In another embodiment, the compound comprises a polypeptide. In another embodiment, the polypeptide comprises non-naturally occurring amino acids. In another embodiment, the polypeptide comprises both (L)- and (D)-amino acids. In another embodiment, the polypeptide comprises at least one (D)-amino acid. In another embodiment the compound comprises an amino acid sequence according to formula I, or an invert thereof, wherein any one or more of the amino acids are optionally non-natural amino acids or (D)-amino acids. In another embodiment, the compound comprises an amino acid sequence according to any one of formulas II-IV, or an invert thereof, wherein any one or more of the amino acids are optionally non-natural amino acids or (D)-amino acids. In another embodiment, the compound comprises an amino acid sequence according to SEQ ID NO.: 1, SEQ ID NO.: 5, SEQ ID NO.: 10, SEQ ID NO.: 66, or SEQ ID NO.: 67, wherein any one or more of the amino acids are optionally non-natural amino acids or (D)-amino acids. In another embodiment, the compound is compound 1, compound 5, compound 10, compound 13, or compound 14. In another embodiment, the polypeptide is not an antibody. In another embodiment, the polypeptide consists of fewer than 30 amino acid residues. In another embodiment, the peptide consists of 60 or fewer amino acid residues. In another embodiment, the polypeptide consists of fewer than 20, fewer than 15, or fewer than 10 amino acid residues. In another embodiment, the polypeptide further comprises a cell-penetrating peptide sequence. In another embodiment, the compound displays at least one of the following characteristics upon binding to RAD51 in a cell: (a) inhibition of assembly of RAD51 monomers on DNA; (b) inhibition of cellular homologous recombination; (c) lack of inhibition of RAD51 ATPase activity; or (d) lowering of RAD51 affinity for Ca2+. In another embodiment, the condition is Bloom's syndrome. In another embodiment, the condition is Bloom's syndrome, Fanconi Anemia, Werner's syndrome, or Nijmegen Breakage syndrome.

In yet a further aspect, the invention relates to a method of reducing drug resistance in a cell, comprising contacting the cell with a polypeptide that binds RADAP1's binding site on human RAD51. In one embodiment the compound interacts with at least one of residues 202, 205, and 206 of human RAD51. In one embodiment, the compound further interacts with residue 187 of human RAD51. In another embodiment, the compound has a binding constant to RAD51 with a Kd value of 10⁻⁴ M or less. In another embodiment, the compound comprises a polypeptide. In another embodiment, the polypeptide comprises non-naturally occurring amino acids. In another embodiment, the polypeptide comprises both (L)- and (D)-amino acids. In another embodiment, the polypeptide comprises at least one (D)-amino acid. In another embodiment the compound comprises an amino acid sequence according to formula I, or an invert thereof, wherein any one or more of the amino acids are optionally non-natural amino acids or (D)-amino acids. In another embodiment, the compound comprises an amino acid sequence according to any one of formulas II-IV, or an invert thereof, wherein any one or more of the amino acids are optionally non-natural amino acids or (D)-amino acids. In another embodiment, the compound comprises an amino acid sequence according to SEQ ID NO.: 1, SEQ ID NO.: 5, SEQ ID NO.: 10, SEQ ID NO.: 66, or SEQ ID NO.: 67, wherein any one or more of the amino acids are optionally non-natural amino acids or (D)-amino acids. In another embodiment, the compound is compound 1, compound 5, compound 10, compound 13, or compound 14. In another embodiment, the polypeptide is not an antibody. In another embodiment, the polypeptide consists of fewer than 30 amino acid residues. In another embodiment, the polypeptide consists of 60 or fewer amino acid residues. In another embodiment, the polypeptide consists of fewer than 20, fewer than 15, or fewer than 10 amino acid residues. In another embodiment, the polypeptide further comprises a cell-penetrating peptide sequence. In another embodiment, the compound displays at least one of the following characteristics upon binding to RAD51 in a cell: (a) inhibition of assembly of RAD51 monomers on DNA; (b) inhibition of cellular homologous recombination; (c) lack of inhibition of RAD51 ATPase activity; or (d) lowering of RAD51 affinity for Ca2+. In another embodiment, the drug is PD-L1, an anti-PD1 agent, or a PARP inhibitor. In another embodiment, the drug is melphalan, doxorubicin, adriamycin, etoposide, camptothecins, mitomycin C, cisplatin, temozolomide, oxaliplatin, carboplatin, or gemcitabine.

In still yet a further aspect, the invention relates to a method for detecting an interaction between a first test protein and a second test protein in a host cell, comprising: a) expressing in the host cell a first fusion protein comprising the first test protein and a DNA-binding moiety; b) expressing in the host cell a second fusion protein comprising the second test protein and a gene activating moiety; and c) expressing in the host cell a death agent, wherein the cytotoxic reporter is activated by a promoter DNA sequence specific for the DNA binding moiety, wherein interaction between the first protein and the second protein causes the gene activating moiety to activate expression of the cytotoxic reporter. In one embodiment, the host cell comprises more than one cytotoxic reporter activated by the promoter DNA sequence specific for the DNA-binding moiety. In another embodiment, the host cell comprises: a) genomic DNA encoding the first fusion protein and genomic DNA encoding the second fusion protein; and b) plasmid DNA encoding the cytotoxic reporter. In another embodiment, the DNA binding moiety is derived from LexA, cI, Glucocorticoid receptor, TetR, or Ume6. In another embodiment, the gene activating moiety is derived from GAL4, B42, VP16 or Dof1. In another embodiment, the cytotoxic reporter is a ribosomally-encoded xenobiotic agent, a ribosomally-encoded poison, a ribosomally-encoded endogenous or exogenous gene that results in severe growth defects upon mild overexpression, a ribosomally-encoded recombinase that excises an essential gene for viability, a limiting factor involved in the synthesis of a toxic secondary metabolite, or any combination thereof. In another embodiment, the cytotoxic reporter is Cholera toxin, SpvB toxin, CARDS toxin, SpyA Toxin, HopU1, Chelt toxin, Certhrax toxin, EFV toxin, ExoT, CdtB, Diphtheria toxin, ExoU/VipB, HopPtoE, HopPtoF, HopPtoG, VopF, YopJ, AvrPtoB, SdbA, SidG, VpdA, Lpg0969, Lpg1978, YopE, SptP, SopE2, SopB/SigD, SipA, YpkA, YopM, Amatoxin, Phallacidin, Killer toxin KP1, Killer toxin KP6, Killer Toxin K1, Killer Toxin K28 (KHR), Killer Toxin K28 (KHS), Anthrax lethal factor endopeptidase, Shiga Toxin, Ricin Toxin, or any combination thereof. In another embodiment, the host cell is fungal or bacterial. In another embodiment, the method further comprises expressing a test gene in the host cell comprising a DNA sequence that encodes a randomized polypeptide library. In another embodiment, the randomized polypeptide library comprises polypeptides 60 or fewer amino acids in length. In another embodiment, the randomized polypeptide library comprises polypeptides 30 or fewer amino acids in length. In another embodiment, the randomized polypeptide library comprises polypeptides 20 or fewer amino acids in length. In another embodiment, the test gene comprises a 3′UTR of a short protein. In another embodiment, the 3′UTR is the 3′UTR of sORF1. In another embodiment, the DNA sequence that encodes a randomized polypeptide library encodes a common N-terminal sequence of Methionine-Valine-Asparagine. In another embodiment, the DNA sequence that encodes a randomized polypeptide library encodes a common N-terminal stabilization sequence. In another embodiment, polypeptides encoded by the randomized peptide library are processed into cyclic peptides in the host cell. In another embodiment, polypeptides encoded by the randomized peptide library are processed into cyclic peptides in the host cell by POPB from G. marginata or A. bisporigera.

In a further aspect, the invention relates to a plasmid vector comprising the nucleotide sequence of SEQ ID NO.: 63. In one embodiment, the plasmid vector has a DNA sequence encoding a first polypeptide inserted in frame with TetR-DBD, and a DNA sequence encoding a second polypeptide inserted in frame with Dof1-AD.

In a further aspect, the invention relates to a host cell, comprising the nucleotide sequence of SEQ ID NO.: 63, or a host cell comprising the nucleotide sequence of SEQ ID NO.: 63 wherein a DNA sequence encoding a first polypeptide is inserted in frame with TetR-DBD, and wherein a DNA sequence encoding a second polypeptide is inserted in frame with Dof1-AD.

In a further aspect, the invention relates to a library of plasmid vectors, each plasmid vector comprising: a) a DNA sequence encoding a different peptide sequence operably linked to a first switchable promoter; and b) a DNA sequence encoding a cytotoxic reporter under control of a second switchable promoter. In one embodiment, the different peptide sequences encode a common N-terminal stabilization sequence. In another embodiment, the DNA sequence encoding a different peptide sequence further encodes a 3′UTR. In another embodiment, the different peptide sequence is 60 or fewer amino acids in length. In another embodiment, the different peptide sequence is 30 or fewer amino acids in length. In another embodiment, the different peptide sequence is 20 or fewer amino acids in length. In another embodiment, the N-terminal stabilization sequence is M-V-A. In another embodiment, the 3′UTR is derived from sORF1. In another embodiment, the different peptide sequences are random. In another embodiment, the different peptide sequences are pre-enriched for binding to a target.

In a further aspect, the invention relates to a library of host cells, each comprising 0 or 1 copy of the plasmid vectors of the plasmid vector library described above.

In a further aspect, the invention relates to a host cell, expressing: a) a first fusion protein comprising a DNA-binding moiety; b) a second fusion protein comprising a gene activating moiety; c) a cytotoxic reporter, wherein the expression of the cytotoxic reporter is under control of a DNA-binding sequence specific for the DNA-binding moiety; and d) an mRNA comprising a nucleotide sequence encoding a polypeptide of 60 or fewer amino acids, wherein the mRNA comprises a 3′UTR 3′ to the polypeptide, and wherein the polypeptide encodes an N-terminal sequence for peptide stabilization. In one embodiment, the host cell is fungal or bacterial. In another embodiment, the host cell is a haploid yeast cell. In another embodiment, the host cell is a diploid yeast cell. In another embodiment, the diploid yeast cell is produced by mating a first host cell comprising DNA sequences encoding the first chimeric gene and the second chimeric gene, to a second host cell comprising DNA sequences encoding the cytotoxic reporter and the mRNA comprising a nucleotide sequence encoding a polypeptide. In another embodiment, the host cell is mammalian.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

Each patent, publication, and non-patent literature cited in the application is hereby incorporated by reference in its entirety as if each was incorporated by reference individually.

### BRIEF DESCRIPTION OF THE FIGURES

The novel features of the disclosure are set forth with particularity in the appended claims. A better understanding of the features and advantages of the present disclosure will be obtained by reference to the following detailed description that sets forth illustrative aspects, in which the principles of the disclosure are utilized, and the accompanying drawings of which:

FIG. 1 depicts the effect of Compounds 1, 3, and 5 on SSP-25 cells.

FIG. 2 depicts the effect of Compounds 1 and 5 on SSP-25 and HeLa cells.

FIG. 3 depicts the effect of Compound 1 on SSP-25 and HeLa cells.

FIG. 4 depicts the effect of Compound 1 on SSP-25 cells.

FIG. 5 depicts the effect of Compounds 1 and 2 on SSP-25 cells.

FIG. 6 depicts the effect of etoposide and Compound 1 on GM08505 cells.

FIG. 7 is an illustrative example of a protein-protein integration plasmid.

FIG. 8 is a sequence for the protein-protein integration plasmid (In the first section of FIG. 8, SEQ ID NO: 60 is disclosed as the top nucleotide sequence and SEQ ID NO: 97 is disclosed as the coded protein. In the second section of FIG. 8, SEQ ID NO: 61 is disclosed as the top nucleotide sequence and SEQ ID NO: 98 is disclosed as the coded protein).

FIG. 9 is an illustrative example of a selection and library plasmid.

FIG. 10 is a sequence for the selection and library plasmid (FIG. 10 discloses the top nucleotide sequence as SEQ ID NO: 62 and the coded protein as SEQ ID NO: 99)

FIG. 11 is an illustrative example of a confirmation plasmid.

FIG. 12 is an illustrative example of a platform of the present disclosure to identify a compound that disrupts a protein-protein interaction.

FIG. 13 shows cell killing of SSP-25 cells using Compound 5 using the xCELLigence assay. The vertical arrows indicate the times of compound administration, and the cell index (Y-axis) represents cell viability.

FIG. 14 compares cell killing of PC3 cells using Compounds 5 and 6 using the xCELLigence assay.

FIG. 15 shows cell killing of A549 cells by Compound 10 using an xCELLigence assay. The vertical line indicates the time of compound administration, and the cell index (Y-axis) represents cell viability

FIG. 16 shows an example of a curve fit to calculate the IC50 for compound 10 based on the data show in in FIG. 15.

FIG. 17 shows the full chemical structure of exemplary compounds according to the invention, alongside measurements of their cell-death IC50 on SSP-25 cells. FIG. 17 discloses SEQ ID NOS 85, 100, 87-89 and 91-96, respectively, in order of appearance.

FIG. 18 shows exemplary compounds according to the invention showing the relative insensitivity of cell-death IC50s to which cell-penetrating peptides are utilized. FIG. 18 discloses SEQ ID NOS 89 and 94-95, respectively, in order of appearance.

FIG. 19 shows the effect of Compound 10 administered intratumorally and intraperitoneally in an in vivo athymic mouse A549 xenograft model.

FIG. 20 shows IC50s of compound 10 in vitro against immortalized cell lines originating from diverse tumor types.

FIG. 21 shows exemplary markush structures encompassing RAD51-interacting compounds as described herein. FIG. 21 discloses SEQ ID NOS 70-73, respectively, in order of appearance.

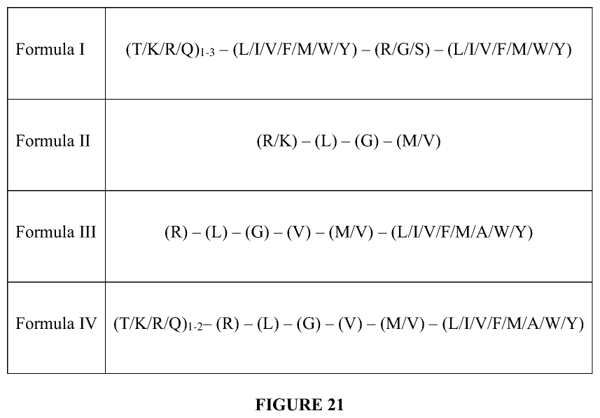

FIG. 22 shows a binding model of an exemplary RAD51 peptide according to the invention bound to RAD51 (as represented by pdb structure 1N0W). Critical residues identified for peptide binding are highlighted. The left panel shows a BRCA2 peptide bound on RAD51 (with the Compound interacting with the highlighted residues around the BRCA2 peptide), the right panel excludes this sequence of BRCA2 and highlights just the Compound interacting residues on RAD51.

FIG. 23 shows a sequence alignment of RAD51 derived from various different species, grouped according to whether RAD51-interacting peptides according to the invention do or do not bind to SEQ ID NO.: 5 as assessed by a Y2H assay using a RAD51/SEQ ID NO.: 5 pair. Highlighted are variant residues that appear to account for the difference in binding. FIG. 23 discloses SEQ ID NOS 101-144, respectively, in order of appearance.

FIG. 24 shows an xCELLigence cell death assay on SSP-25 cells, wherein compound 10 is added alone or combined with the calcium chelator BAPTA-AM, demonstrating that addition of BAPTA-AM rescues cell death due to compound 10.

FIG. 25 shows an xCELLigence cell death assay on SSP-25 cells, wherein compound 10 is added alone or combined with the calcium chelator ruthenium red, demonstrating that addition of ruthenium red rescues cell death due to compound 10.

FIG. 26 shows an xCELLigence cell death assay on SSP-25 cells, wherein compound 10 or olaparib are added alone or in combination, demonstrating that the combination of compound 10 and olaparib enhances cell death relative to compound 10 or olaparib alone.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The present disclosure provides methods for the treatment of cancer using compounds that can bind to proteins involved in the homologous recombination DNA repair pathway. The present compounds can decrease the rate of cellular proliferation in cancer cells, and avoid affecting those cells that do not overexpress proteins specific to the DNA repair pathway. The present compounds can further sensitize cells to chemotherapy, and sensitize those cells that have developed resistance to therapeutics. The compounds disclosed herein can display specificity toward cancer cells harboring specific transcriptional signatures.

Cancer is a collection of related diseases characterized by uncontrolled proliferation of cells with the potential to metastasize throughout the body. Cancer can be classified into five broad categories including, for example, carcinomas, sarcomas, lymphomas, leukemias, and adenomas. Carcinomas can arise from cells that cover internal and external parts of the body such as the lung, breast, and colon. Sarcomas can arise from cells that are located in bone, cartilage, fat, connective tissue, muscle, and other supportive tissues. Lymphomas can arise in the lymph nodes and immune system tissues. Leukemias can arise in the bone marrow and accumulate in the bloodstream. Adenomas can arise in the thyroid, the pituitary gland, the adrenal gland, and other glandular tissues.

Although different cancers can develop in virtually any of the body's tissues, the basic processes that cause cancer can be similar in all forms of the disease. Cancer begins when a cell breaks free from the normal restraints on cell division and begins to grow and divide abnormally. Genetic mutations in the cell can preclude the ability of the cell to control cell division or initiate apoptosis, and can result in uncontrolled growth and division of cells.

Oncogenes and tumor suppressor genes can regulate the proliferation of cells. Genetic mutations can affect oncogenes and tumor suppressors, potentially activating or suppressing activity abnormally, further facilitating uncontrolled cell division. Whereas oncogenes assist in cellular growth, tumor suppressor genes slow cell division by repairing damaged DNA and activating apoptosis. Cellular oncogenes that can be mutated in cancer include, for example, Cdk1, Cdk2, Cdk3, Cdk4, Cdk6, c-MYC, EGFR, HER2, K-Ras, PDGFR, Raf kinase, and VEGF. Tumor suppressor genes that can be mutated in cancer include, for example, BRCA1, BRCA2, cyclin-dependent kinase inhibitor 1C, PTEN, p16, p27, p53, p73, and Retinoblastoma protein (pRb).

### DNA Damage and Cancer

DNA damage can occur as a result of, for example, UV radiation, IR radiation, X-rays, reactive oxygen species, depurination, depyrimidination, single-strand breaks, double-strand breaks, cytosine deamination, O6-methylguanines, base alkylation, cross-linking of DNA, replication errors, or free radicals. Chemical compounds can also cause DNA damage by causing bulky adducts, interstrand crosslinks, intrastrand crosslinks, intercalation between DNA strands, or DNA alkylation. Compounds that can cause DNA damage include, for example, actinomycin-D, benzo[α]pyrenes, cisplatin, daunorubicin, ethidium bromide, nitrogen mustards, methyl methanesulphonate (MMS), N-ethyl-N-nitrosourea (ENU), N-nitroso-N-methylurea (NMU), or psoralens.

Mutations or precocious expression of the DNA damage repair pathway can be found in cancer. Genes that can be affected in the DNA damage repair pathway include, for example, ATM, ATRX, BRCA1, BRCA2, ERCC1, FANCB, FANCF, FEN1, HMGA1, HMGA1, MDC1, MGMT, MLH1, MSH2, MSH4, Mre11A, NBS1, NEIL1, PARP1, PARP2, PMS2, RAD51, RAD52, RAD54, RAD51AP1, WRN, or XPF.

BRCA1 and BRCA2 are tumor suppressors that are involved in the cellular DNA damage repair pathway. Both BRCA1 and BRCA2 can interact with RAD51, a eukaryotic recombinase involved in DNA repair. Germline mutations in BRCA1 or BRCA2 can predispose individuals to various cancers including, for example, breast, ovarian, prostate, lung, and liver cancers. Tumors with BRCA2 mutations can exhibit loss of heterozygosity of the wild-type allele.

BRCA1 can combine with other tumor suppressors, DNA damage sensors, and signal transducers to form a large multi-subunit protein complex known as the BRCA1-associated genome surveillance complex (BASC). BRCA1 can also associate with RNA polymerase II and histone deacetylase complexes. Thus, BRCA1 can play a role in transcription, DNA repair of double-stranded breaks, and recombination. BRCA1 has cell-cycle dependent localization and can be found in, for the example, the nucleus, cytoplasm, or endoplasmic reticulum.

BRCA2 can maintain genome stability, and both BRCA1 and BRCA2 can specifically regulate the homologous recombination pathway for double-strand DNA repair. The BRCA2 protein contains about seven copies of a 70 amino acid motif known as the BRC motif, which can mediate binding to the RAD51 recombinase. RAD51 can perform certain biochemical activities required for homologous recombination and DNA repair, for example, promotion of joint molecule formation and DNA strand exchange between homologous DNA molecules. As a prerequisite for these functions, RAD51 can bind to DNA to form highly ordered nucleoprotein filaments in which the DNA is encased within a protein sheath. RAD51AP1 is a RAD51 accessory protein that can stimulates joint molecule formation through the combination of structure-specific DNA binding and physical contact with RAD51. RAD51AP1 can protect cells from the adverse effects of DNA double-strand break-inducing agents.

Direct and specific interactions between the BRC3 or BRC4 repeats in BRCA2 and RAD51 can sequester RAD51 in a form that is ready to be localized to sites of DNA damage, and thus become activated for DNA repair. Lack of functional BRCA2, or overexpression of BRCA2, can perturb RAD51 function by, for example, preventing RAD51 from localizing to sites of DNA damage. These damaged sites, which can contain double-strand breaks formed at stalled or broken replication forks, or double-strand breaks induced by exogenous agents, can provide the signal for activation of the mammalian SOS repair response. Activation can involve the posttranslational modification of RAD51 or occur via interactions with other repair proteins.

The BRC motifs of BRCA2 can bind monomeric or oligomeric forms of RAD51 in a cell cycle-dependent manner and in response to DNA damage. BRCA2 protein can be directly involved in the nuclear transport of RAD51. For example, the pancreatic adenocarcinoma cell line CAPAN-1 is defective in BRCA2, which can lead to impaired nuclear transportation of RAD51 in CAPAN-1. Thus, RAD51 can require BRCA2 for nuclear translocation and proper homologous recombination processes.

Double-strand DNA breaks can be caused by, for example, natural and medical radiation and other environmental exposures. Double-strand DNA breaks can also occur when chromosomes exchange genetic material during meiosis and during repair of DNA crosslinks. By repairing DNA, BRCA1 and BRCA1 play a role in maintaining the stability of the human genome and reducing the likelihood of dangerous gene rearrangements that can lead to malignancies.

Cancer treatments using chemotherapy or radiotherapy can target and disrupt the function of the DNA of tumor cells by inducing adducts or DNA double-strand or single-strand breaks. Cancer cells can overcome these therapies by developing resistance mechanisms, which can either be induced or intrinsic to the cancer cells. A high level of homologous recombination can be present in cancer cells due to the overexpression of RAD51. This overexpression of RAD51 can be seen in, for example, breast cancer, pancreatic, NSCLC, mCRPC, AML, ICC, and CML. In these cancer cells, the overexpression of RAD51 can provide cancer resistance by promoting the repair of double strand breaks induced by chemotherapy. Thus, the compounds of the present invention can interfere with the activity of RAD51, or other proteins involved in the DNA damage repair pathway, to resensitize cancer cells to chemotherapy, or to potentiate the effect of chemotherapy.

### Two-Hybrid System

Two-hybrid screening can be used to identify and characterize protein-protein interactions. The two-hybrid system was initially developed using yeast as a host organism. However, bacterial two-hybrid systems can also be used to characterize protein-protein interactions. The present disclosure provides a system that can use a unified yeast and bacterial two-hybrid system in which a single bait expression plasmid is used in both organismal contexts. Additionally, an extensive series of leucine zipper fusion proteins of known affinities can be generated to compare the efficiency of interaction detection using both systems. The yeast system can produce a quantitative readout over a dynamic range. “Auto-activation” by baits can be less prevalent in the bacterial system. In addition, modified expression vectors disclosed herein can be used for expression of a protein of interest in both yeast and bacteria.

As used herein, “reporter gene” refers to a gene whose expression can be assayed. Such genes include, for example, LacZ, β-glucuronidase (GUS), amino acid biosynthetic genes, the yeast LEU2, HIS3, LYS2, or URA3 genes, nucleic acid biosynthetic genes, the mammalian chloramphenicol transacetylase (CAT) gene, the green fluorescent protein (GFP) or any surface antigen gene for which specific antibodies are available.

A “promoter” is a DNA sequence located proximal to the start of transcription at the 5′ end of an operably linked transcribed sequence. The promoter can contain one or more regulatory elements or modules, which interact in modulating transcription of the operably linked gene. Promoters can come be switchable or constitutive. Switchable promoters allow for reversible induction or repression of operably linked target genes upon administration of an agent. Examples of switchable promoters include but are not limited to the TetO operator and the alcohol dehydrogenase I (alcA) gene promoter. Examples of constitutive promoters include the human beta-actin gene promoter.

“Operably linked” describes two macromolecular elements arranged such that modulating the activity of the first element induces an effect on the second element. In this manner, modulation of the activity of a promoter element can be used to alter or regulate the expression of an operably-linked coding sequence. For example, the transcription of a coding sequence that is operably-linked to a promoter element is induced by factors that activate the promoter's activity; transcription of a coding sequence that is operably-linked to a promoter element is inhibited by factors that repress the promoter's activity. Thus, a promoter region is operably-linked to the coding sequence of a protein if transcription of such coding sequence activity is influenced by the activity of the promoter.

“In frame” as used herein throughout, refers to the proper positioning of a desired sequence of nucleotides within a DNA fragment or coding sequence operably linked to a promoter sequence that results in optimal transcription or translation.

“Fusion construct” refers to recombinant genes that encode fusion proteins.

A “fusion protein” is a hybrid protein, i.e., a protein that has been constructed to contain domains from at least two different proteins. As used herein, a fusion protein is a hybrid protein that possesses (a) transcriptional regulatory domain from a transcriptional regulatory protein, or (b) a DNA binding domain from a DNA binding protein linked to a heterologous protein to be assayed for interaction. The structure of the fusion protein is such that the transcriptional regulatory domain and the DNA binding domain are arranged in a manner that allows both domains to be biologically active. The protein that is the source of the transcriptional regulatory domain is different from the protein that is the source of the DNA binding domain. In other words, the two domains are heterologous to each other.

The transcriptional regulatory domain of the fusion protein can either activate or repress transcription of target genes, depending on the native biological activity of the domain. The bait proteins of the invention are also fusion proteins encoded by a fusion gene that can contain a protein of interest operably linked to a DNA binding moiety.

The term “fusion protein gene” refers to a DNA sequence which encodes a fusion protein. A fusion protein gene can further provide transcriptional and translational regulatory elements for the transcriptional and translational control thereof.

“Expression” is the process by which the information encoded within a gene is revealed. If the gene encodes a protein, then expression involves both transcription of the DNA into mRNA, the processing of mRNA (if necessary) into a mature mRNA product, and translation of the mature mRNA into protein.

A nucleic acid molecule, such as a DNA or gene is said to be “capable of expressing” a polypeptide if the molecule contains the coding sequences for the polypeptide and the expression control sequences that, in the appropriate host environment, provide the ability to transcribe, process and translate the genetic information contained in the DNA into a protein product, and if such expression control sequences are operably-linked to the nucleotide sequence that encodes the polypeptide.

As used herein, a “cloning vehicle” is any entity that is capable of delivering a nucleic acid sequence into a host cell for cloning purposes. Examples of cloning vehicles include plasmids or phage genomes. A plasmid that can replicate autonomously in the host cell is especially desired. Alternatively, a nucleic acid molecule that can insert (integrate) into the host cell's chromosomal DNA is useful, especially a molecule that inserts into the host cell's chromosomal DNA in a stable manner, that is, a manner that allows such molecule to be inherited by daughter cells.

A “host cell” as described herein can be a bacterial, fungal, or mammalian cell. Examples of bacterial host cells are E. coli and B. subtilis Examples of fungal cells are S. cerevisiae and S. pombe. Non-limiting examples of mammalian cells are immortalized mammalian cell lines, such as HEK293, A549, HeLa, or CHO cells, or isolated patient primary tissue cells that have been genetically immortalized (such as by transfection with hTERT).

Cloning vehicles are often characterized by one or a small number of endonuclease recognition sites at which such DNA sequences may be cut in a determinable fashion without loss of an essential biological function of the vehicle, and into which DNA may be spliced in order to bring about its replication and cloning.

The cloning vehicle can further contain a marker suitable for use in the identification of cells transformed with the cloning vehicle. For example, a marker gene can be a gene that confers resistance to a specific antibiotic on a host cell.

The word “vector” can be used interchangeably with “cloning vehicle”.

As used herein, an “expression vehicle” is a vehicle or vector similar to the cloning vehicle but is especially designed to provide an environment that allows the expression of the cloned gene after transformation into the host. One manner of providing such an environment is to include transcriptional and translational regulatory sequences on such expression vehicles, such transcriptional and translational regulatory sequences being capable of being operably linked to the cloned gene. Another manner of providing such an environment is to provide a cloning site or sites on such vehicle, wherein a desired cloned gene and a desired expression regulatory element can be cloned.

In an expression vehicle, the gene to be cloned is usually operably-linked to certain control sequences such as promoter sequences. Expression control sequences will vary depending on whether the vector is designed to express the operably-linked gene in a prokaryotic or eukaryotic host and can additionally contain transcriptional elements such as enhancer elements, termination sequences, tissue-specificity elements, or translational initiation and termination sites.

A “host” refers to any organism that is the recipient of a cloning or expression vehicle. The host may be a yeast cell or a cultured animal cell such as a mammalian or insect cell. The yeast host may be Saccharomyces cerevisiae.

A “binding moiety” or a “DNA-binding moiety” is a stretch of amino acids that is capable of directing specific polypeptide binding to a particular DNA sequence (i.e., a “protein binding site”). Also referred to herein as a DNA binding domain, these proteins can be homodimers or monomers that bind DNA in a sequence specific manner. Exemplary DNA binding domains of the invention include LexA, cI, glucocorticoid receptor binding domains, and the Ume6 domain.

A “gene activating moiety” is a stretch of amino acids that is capable of weakly inducing the expression of a gene to whose control region it is bound (one example is an activation domain from a transcription factor). As used herein, “weakly” is meant below the level of activation effected by GAL4 activation region II and is preferably at or below the level of activation effected by the B42 activation domain. Levels of activation can be measured using any downstream reporter gene system and comparing, in parallel assays, the level of expression stimulated by the GAL4 region II-polypeptide with the level of expression stimulated by the polypeptide to be tested.

### Screening of Protein-Protein Interaction Inhibitors

The large and broad surfaces that form the contact interface between two proteins can be potential targets of canonical small molecule inhibitors. However, the large and broad surfaces can have size limitations, and evolved resistance can occur readily. The specificity of antibodies can be combined with cell permeability in the form of short peptides, for example, peptides of less than 25 residues. Screening for short peptide blockers of protein-protein interaction (PPIs) can be performed using technologies such as phage display or mRNA display; however, these screens are performed in vitro and can require the purification of one of the interacting proteins of interest. Upon selection of a peptide sequence with affinity toward one of the proteins, secondary screens can be performed to validate that the peptide interferes with the binding interface of the second protein. This secondary screening can further rely upon the proper folding of the proteins and the replication of intracellular biophysical conditions in the assays.

A method of the invention can involve the intracellular selection of potent peptide disruptors of PPIs. A model organism, for example, Saccharomyces cerevisiae, can be employed, and the coexpression of a PPI of interest with a test gene comprising a DNA sequence that encodes randomized peptide library can allow the selection of unbiased peptides that interfere with a PPI using stringent selection mechanisms. The method can begin with a permutation of a yeast two-hybrid system that can rely on the reconstitution of a transcription factor that requires an interaction between a first test protein fused to a DNA binding domain (DBD) and a second test protein fused to a transcription activation domain (AD) or gene activating moiety.

An efficient interaction between the two proteins of interest can direct RNA polymerase to a specific genomic site, and allow for the expression of a genetic element. The genetic element can be, for example, a gene that encodes a protein that enables an organism to grown on selection media. The selection media can be specific to, for example, ADE2, URA3, TRP1, KAN^{R}, or NAT^{R}. Markers that can detect when an interaction is no longer present, for example, disrupted by an external object, can be referred to as counter-selection markers, such as the URA3 gene, and can be poor or leaky (easily masked by the selection of mutants that escape the selection). This leakiness of the selection marker can lead to a high false positive rate.

A method of the invention can combine a strong negative selection marker with the intracellular stabilization of the production of short peptides to screen for blockers of PPIs. An inducible two-hybrid approach can be employed, which can drive the expression of any one or combination of several cytotoxic reporters (death agents). A method of the invention involving induced expression of a combination of cytotoxic reporters in a two-hybrid system can allow for a multiplicative effect in lowering the false-positive rate of the two-hybrid assay, as all of the cytotoxic reporters must simultaneously be “leaky” to allow for an induced cell to survive, The cytotoxic reporters can be, for example:

TABLE 1

| | | |
|---|---|---|
| Cholera | SEQ ID | MVKIIFVFFIFLSSFSYANDDKLYRADSRPPDEIKQSGGLMPRG |
| toxin | NO.: 20 | QSEYFDRGTQMNINLYDHARGTQTGFVRHDDGYVSTSISLRS |
| (CtxA) | | AHLVGQTILSGHSTYYIYVIATAPNMFNVNDVLGAYSPHPDE |
| | | QEVSALGGIPYSQIYGWYRVHFGVLDEQLHRNRGYRDRYYS |
| | | NLDIAPAADGYGLAGFPPEHRAWREEPWIHHAPPGCGNAPRS |
| | | SMSNTCDEKTQSLGVKFLDEYQSKVKRQIFSGYQSDIDTHNRI |
| | | KDEL |
| SpvB | SEQ ID | MLILNGFSSATLALITPPFLPKGGKALSQSGPDGLASITLPLPIS |
| toxin | NO.: 21 | AERGFAPALALHYSSGGGNGPFGVGWSCATMSIARRTSHGVP |
| (Salmonella | | QYNDSDEFLGPDGEVLVQTLSTGDAPNPVTCFAYGDVSFPQS |
| enterica) | | YTVTRYQPRTESSFYRLEYWVGNSNGDDFWLLHDSNGILHLL |
| | | GKTAAARLSDPQAASHTAQWLVEESVTPAGEHIYYSYLAENG |
| | | DNVDLNGNEAGRDRSAMRYLSKVQYGNATPAADLYLWTSA |
| | | TPAVQWLFTLVFDYGERGVDPQVPPAFTAQNSWLARQDPFSL |
| | | YNYGFEIRLHRLCRQVLMFHHFPDELGEADTLVSRLLLEYDE |
| | | NPILTQLCAARTLAYEGDGYRRAPVNNMMPPPPPPPPPMMGG |
| | | NSSRPKSKWAIVEESKQIQALRYYSAQGYSVINKYLRGDDYP |
| | | ETQAKETLLSRDYLSTNEPSDEEFKNAMSVYINDIAEGLSSLPE |
| | | TDHRVVYRGLKLDKPALSDVLKEYTTIGNIIIDKAFMSTSPDK |
| | | AWINDTILNIYLEKGHKGRILGDVAHFKGEAEMLFPPNTKLKI |
| | | ESIVNCGSQDFASQLSKLRLSDDATADTNRIKRIINMRVLNS |
| CARDS | SEQ ID | MSENLYFQGHMPNPVRFVYRVDLRSPEEIFEHGFSTLGDVRN |
| toxin | NO.: 22 | FFEHILSTNFGRSYFISTSETPTAAIRFFGSWLREYVPEHPRRAY |
| (Mycoplasma | | LYEIRADQHFYNARATGENLLDLMRQRQVVFDSGDREMAQ |
| pneumoniae) | | MGIRALRTSFAYQREWFTDGPIAAANVRSAWLVDAVPVEPG |
| | | HAHHPAGRVVETTRINEPEMHNPHYQELQTQANDQPWLPTP |
| | | GIATPVHLSIPQAASVADVSEGTSASLSFACPDWSPPSSNGENP |
| | | LDKCIAEKIDNYNLQSLPQYASSVKELEDTPVYLRGIKTQKTF |
| | | MLQADPQNNNVFLVEVNPKQKSSFPQTIFFWDVYQRICLKDL |
| | | TGAQISLSLTAFTTQYAGQLKVHLSVSAVNAVNQKWKMTPQ |
| | | DIAITQFRVSSELLGQTENGLFWNTKSGGSQHDLYVCPLKNPP |
| | | SDLEELQIIVDECTTHAQFVTMRAASTFFVDVQLGWYWRGY |
| | | YYTPQLSGWSYQMKTPDGQIFYDLKTSKIFFVQDNQNVFFLH |
| | | NKLNKQTGYSWDWVEWLKHDMNEDKDENFKWYFSRDDLTI |
| | | PSVEGLNFRHIRCYADNQQLKVIISGSRWGGWYSTYDKVESN |
| | | VEDKILVKDGFDRF |
| SpyA | SEQ ID | MLKKRYQLAMILLLSCFSLVWQTEGLVELFVCEHYERAVCEG |
| Toxin | NO.: 23 | TPAYFTFSDQKGAETLIKKRWGKGLVYPRAEQEAMAAYTCQ |
| (Streptococcus | | QAGPINTSLDKAKGKLSQLTPELRDQVAQLDAATHRLVIPWN |
| pyogenes) | | IVVYRYVYETFLRDIGVSHADLTSYYRNHQFNPHILCKIKLGT |
| | | RYTKHSFMSTTALKNGAMTHRPVEVRICVKKGAKAAFVEPY |
| | | SAVPSEVELLFPRGCQLEVVGAYVSQDHKKLHIEAYFKGSL |
| HopU1 | SEQ ID | MNINRQLPVSGSERLLTPDVGVSRQACSERHYSTGQDRHDFY |
| (Pseudomonas | NO.: 24 | RFAARLHVDAQCFGLSIDDLMDKFSDKHFRAEHPEYRDVYPE |
| syringae) | | ECSAIYMHTAQDYSSHLVRGEIGTPLYREVNNYLRLQHENSG |
| | | REAEIDNHDEKLSPHIKMLSSALNRLMDVAAFRGTVYRGIRG |
| | | DLDTIARLYHLFDTGGRYVEPAFMSTTRIKDSAQVFEPGTPNN |
| | | IAFQISLKRGADISGSSQAPSEEEIMLPMMSEFVIEHASALSEGK |
| | | HLFVLSQI |
| Chelt | SEQ ID | MKTIISLIFIMFPLFVSAHNGNFYRADSRSPNEIKDLGGLYPRG |
| toxin | NO.: 25 | YYDFFERGTPMSISLYDHARGAPSGNTRYDDGFVSTTTDIDSA |
| | | HEIGQNILSGYTEYYIYLIAPAPNLLDVNAVLGRYSPHPQENE |
| | | YSALGGIPWTQVIGWYVVNNGVLDRNIHRNRQFRADLFNNLS |
| | | PALPSESYQFAGFEPEHPAWRQEPWINFAPPGCGRNVRLTKHI |
| | | NQQDCSNSQEELVYKKLQDLRTQFKVDKKLKLVNKTSSNNII |
| | | FPNHDFIREWVDLDGNGDLSYCGFTVDSDGSRKRIVCAHNNG |
| | | NFTYSSINISLSDYGWPKGQRFIDANGDGLVDYCRVQYVWTH |
| | | LYCSLSLPGQYFSLDKDAGYLDAGYNNSRAWAKVIGTNKYS |
| | | FCRLTSNGYICTDIDSYSTAFKDDDQGWADSRYWMDIDGNG |
| | | GDDYCRLVYNWTHLRCNLQGKDGLWKRVESKYLDGGYPSL |
| | | RFKIKMTSNKDNYCRIVRNHRVMECAYVSDNGEFHNYSLNM |
| | | PFSLYNKNDIQFIDIDGDNRDDICRYNSAPNTMECYLNQDKSF |
| | | SQNKLVLYLSAKPISSLGSGSSKIIRTFNSEKNSSAYCYNAGYG |
| | | TLRCDEFVIY |
| Certhrax | SEQ ID | MKEIIRNLVRLDVRSDVDENSKKTQELVEKLPHEVLELYKNV |
| toxin | NO.: 26 | GGEIYITDKRLTQHEELSDSSHKDMFIVSSEGKSFPLREHFVFA |
| | | KGGKEPSLIIHAEDYASHLSSVEVYYELGKAIIRDTFPLNQKEL |
| | | GNPKFINAINEVNQQKEGKGVNAKADEDGRDLLFGKELKKN |
| | | LEHGQLVDLDLISGNLSEFQHVFAKSFALYYEPHYKEALKSY |
| | | APALFNYMLELDQMRFKEISDDVKEKNKNVLDFKWYTRKAE |
| | | SWGVQTFKNWKENLTISEKDIITGYTGSKYDPINEYLRKYDGE |
| | | IIPNIGGDLDKKSKKALEKIENQIKNLDAALQKSKITENLIVYR |
| | | RVSELQFGKKYEDYNLRQNGIINEEKVMELESNFKGQTFIQHN |
| | | YMSTSLVQDPHQSYSNDRYPILLEITIPEGVHGAYIADMSEYP |
| | | GQYEMLINRGYTFKYDKFSIVKPTREEDKGKEYLKVNLSIYLG |
| | | NLNREK |
| EFV | SEQ ID | MSQLNKWQKELQALQKANYQETDNQLFNVYRQSLIDIKKRL |
| toxin | NO.: 27 | KVYTENAESLSFSTRLEVERLFSVADEINAILQLNSPKVEKTIK |
| | | GYSAKQAEQGYYGLWYTLEQSQNIALSMPLINHDYIMNLVN |
| | | APVAGKRLSKRLYKYRDELAQNVTNNIITGLFEGKSYAEIAR |
| | | WINEETEASYKQALRIARTEAGRTQSVTTQKGYEEAKELGINI |
| | | KKKWLATIDKHTRRTHQELDGKEVDVDEEFTIRGHSAKGPR |
| | | MFGVASEDVNCRCTTIEVVDGISPELRKDNESKEMSEFKSYDE |
| | | WYADRIRQNESKPKPNFTELDFFGQSDLQDDSDKWVAGLKPE |
| | | QVNAMKDYTSDAFAKMNKILRNEKYNPREKPYLVNIIQNLD |
| | | DAISKFKLKHDIITYRGVSANEYDAILNGNVFKEFKSTSINKKV |
| | | AEDFLNFTSANKDGRVVKFLIPKGTQGAYIGTNSSMKKESEFL |
| | | LNRNLKYTVEIVDNILEVTILG |
| ExoT | SEQ ID | MHIQSSQQNPSFVAELSQAVAGRLGQVEARQVATPREAQQLA |
| | NO.: 28 | QRQEAPKGEGLLSRLGAALARPFVAIIEWLGKLLGSRAHAAT |
| | | QAPLSRQDAPPAASLSAAEIKQMMLQKALPLTLGGLGKASEL |
| | | ATLTAERLAKDHTRLASGDGALRSLATALVGIRDGSLIEASRT |
| | | QAARLLEQSVGGIALQQWGTAGGAASQHVLSASPEQLREIAV |
| | | QLHAVMDKVALLRHAVESEVKGEPVDKALADGLVEHFGLEA |
| | | EQYLGEHPDGPYSDAEVMALGLYTNGEYQHLNRSLRQGREL |
| | | DAGQALIDRGMSAAFEKSGPAEQVVKTFRGTQGRDAFEAVK |
| | | EGQVGHDAGYLSTSRDPSVARSFAGLGTITTLFGRSGIDVSEIS |
| | | IEGDEQEILYDKGTDMRVLLSAKDGQGVTRRVLEEATLGERS |
| | | GHSEGLLDALDLATGTDRSGKPQEQDLRLRMRGLDLA |
| CdtB | SEQ ID | MKKIICLFLSFNLAFANLENFNVGTWNLQGSSAATESKWSVS |
| | NO.: 29 | VRQLVSGANPLDILMIQEAGTLPRTATPTGRHVQQGGTPIDEY |
| | | EWNLGTLSRPDRVFIYYSRVDVGANRVNLAIVSRMQAEEVIV |
| | | LPPPTTVSRPIIGIRNGNDAFFNIHALANGGTDVGAIITAVDAH |
| | | FANMPQVNWMIAGDFNRDPSTITSTVDRELANRIRVVFPTSAT |
| | | QASGGTLDYAITGNSNRQQTYTPPLLAAILMLASLRSHIVSDH |
| | | FPVNFRKF |
| Diphtheria | SEQ ID | MSRKLFASILIGALLGIGAPPSAHAGADDVVDSSKSFVMENFS |
| toxin | NO.: 30 | SYHGTKPGYVDSIQKGIQKPKSGTQGNYDDDWKGFYSTDNK |
| | | YDAAGYSVDNENPLSGKAGGVVKVTYPGLTKVLALKVDNA |
| | | ETIKKELGLSLTEPLMEQVGTEEFIKRFGDGASRVVLSLPFAEG |
| | | SSSVEYINNWEQAKALSVELEINFETRGKRGQDAMYEYMAQ |
| | | ACAGNRVRRSVGSSLSCINLDWDVIRDKTKTKIESLKEHGPIK |
| | | NKMSESPNKTVSEEKAKQYLEEFHQTALEHPELSELKTVTGT |
| | | NPVFAGANYAAWAVNVAQVIDSETADNLEKTTAALSILPGIG |
| | | SVMGIADGAVHHNTEEIVAQSIALSSLMVAQAIPLVGELVDIG |
| | | FAAYNFVESIINLFQVVHNSYNRPAYSPGHKTQPFLHDGYAVS |
| | | WNTVEDSIIRTGFQGESGHDIKITAENTPLPIAGVLLPTIPGKLD |
| | | VNKSKTHISVNGRKIRMRCRAIDGDVTFCRPKSPVYVGNGVH |
| | | ANLHVAFHRSSSEKIHSNEISSDSIGVLGYQKTVDHTKVNSKL |
| | | SLFFEIKS |
| ExoU/VipB | SEQ ID | MKLAEIMTKSRKLKRNLLEISKTEAGQYSVSAPEHKGLVLSG |
| | NO.: 31 | GGAKGISYLGMIQALQERGKIKNLTHVSGASAGAMTASILAV |
| | | GMDIKDIKKLIEGLDITKLLDNSGVGFRARGDRFRNILDVIYM |
| | | MQMKKHLESVQQPIPPEQQMNYGILKQKIALYEDKLSRAGIVI |
| | | NNVDDIINLTKSVKDLEKLDKALNSIPTELKGAKGEQLENPRL |
| | | TLGDLGRLRELLPEENKHLIKNLSVVVTNQTKHELERYSEDTT |
| | | PQQSIAQVVQWSGAHPVLFVPGRNAKGEYIADGGILDNMPEI |
| | | EGLDREEVLCVKAEAGTAFEDRVNKAKQSAMEAISWFKARM |
| | | DSLVEATIGGKWLHATSSVLNREKVYYNIDNMIYINTGEVTTT |
| | | NTSPTPEQRARAVKNGYDQTMQLLDSHKQTFDHPLMAILYIG |
| | | HDKLKDALIDEKSEKEIFEASAHAQAILHLQEQIVKEMNDGD |
| | | YSSVQNYLDQIEDILTVDAKMDDIQKEKAFALCIKQVNFLSEG |
| | | KLETYLNKVEAEAKAAAEPSWATKILNLLWAPIEWVVSLFKG |
| | | PAQDFKVEVQPEPVKVSTSENQETVSNQKDINPAVEYRKIIAE |
| | | VRREHTDPSPSLQEKERVGLSTTFGGH |
| HopPtoE | SEQ ID | MNRVSGSSSATWQAVNDLVEQVSERTTLSTTGYQTAMGRLN |
| | NO.: 32 | KPEKSDADALMTMRRAQQYTDSAKRTYISETLMNLADLQQR |
| | | KIYRTNSGNLRGAIEMTPTQLTDCVQKCREEGFSNCDIQALEI |
| | | GLHLRHKLGISDFTIYSNRKLSHNYVVIHPSNAFPKGAIVDSW |
| | | TGQGVVELDFKTRLKFKHREENYAVNANMHEWIERYGQAH |
| | | VID |
| HopPtoF | SEQ ID | MGNICGTSGSRHVYSPSHTQRITSAPSTSTHVGGDTLTSIHQLS |
| | NO.: 33 | HSQREQFLNMHDPMRVMGLDHDTELFRTTDSRYIKNDKLAG |
| | | NPQSMASILMHEELRPNRFASHTGAQPHEARAYVPKRIKATD |
| | | LGVPSLNVMTGSLARDGIRAYDHMSDNQVSVKMRLGDFLER |
| | | GGKVYADASSVADDGETSQALIVTLPKGQKVPVERV |
| HopPtoG | SEQ ID | MQIKNSHLYSASRMVQNTFNASPKMEVTNAIAKNNEPAALSA |
| | NO.: 34 | TQTAKTHEGDSKGQSSNNSKLPFRAMRYAAYLAGSAYLYDK |
| | | TANNFFLSTTSLHDGKGGFTSDARLNDAQDKARKRYQNNHS |
| | | STLENKNSLLSPLRLCGENQFLTMIDYRAATKIYLSDLVDTEQ |
| | | AHTSILKNIMCLKGELTNEEAIKKLNPEKTPKDYDLTNSEAYIS |
| | | KNKYSLTGVKNEETGSTGYTSRSITKPFVEKGLKHFIKATHGE |
| | | KALTPKQCMETLDNLLRKSITLNSDSQFAAGQALLVFRQVYA |
| | | GEDAWGDAERVILKSHYNRGTVLQDEADKIELSRPFSEQDLA |
| | | KNMFKRNTSIAGPVLYHAYIYIQEKIFKLPPDKIEDLKHKSMA |
| | | DLKNLPLTHVKLSNSGVGFEDASGLGDSFTALNATSCVNHAR |
| | | IMSGEPPLSKDDVVILIGCLNAVYDNSSGIRHSLREIARGCFVG |
| | | AGFTVQDGDDFYKQICKNASKQFYNG |
| VopF | SEQ ID | MFKISVSQQANVMSTSDTAQRSSLKISIKSICNKSLSKKLHTLA |
| | NO.: 35 | EKCRRFSQELKEHTASKKQIVEQATTTVRESSLTKSDSELGSS |
| | | RSLLTSDVLSSSSSHEDLTAVNLEDNDSVFVTIESSSELIVKQD |
| | | GSIPPAPPLPGNIPPAPPLPSAGNIPTAPGLPKQKATTESVAQTS |
| | | DNRSKLMEEIRQGVKLRATPKSSSTEKSASDPHSKLMKELINH |
| | | GAKLKKVSTSDIPVPPPLPAAFASKPTDGRSALLSEIAGFSKDR |
| | | LRKAGSSETLNVSQPTVAESSIPEAYDLLLSDEMFNLSPKLSET |
| | | ELNTLADSLADYLFKAADIDWMQVIAEQTKGSTQATSLKSQL |
| | | EQAPEYVKAFCDEILKFPDCYKSADVASPESPKAGPSSVIDVA |
| | | LKRLQAGRNRLFSTIDAKGTNELKKGEAILESAINAARSVMTA |
| | | EQKSALLSSNVKSATFKVFSELPCMEGFAEQNGKAAFNALRL |
| | | AFYSSIQSGDTAQQDIARFMKENLATGFSGYSYLGLTSRVAQL |
| | | EAQLAALTTK |
| YopJ | SEQ ID | MIGPISQINISGGLSEKETSSLISNEELKNIITQLETDISDGSWFH |
| | NO.: 36 | KNYSRMDVEVMPALVIQANNKYPEMNLNLVTSPLDLSIEIKN |
| | | VIENGVRSSRFIINMGEGGIHFSVIDYKHINGKTSLILFEPANFN |
| | | SMGPAMLAIRTKTAIERYQLPDCHFSMVEMDIQRSSSECGIFSF |
| | | ALAKKLYIERDSLLKIHEDNIKGILSDGENPLPHDKLDPYLPVT |
| | | FYKHTQGKKRLNEYLNTNPQGVGTVVNKKNETIVNRFDNNK |
| | | SIVDGKELSVSVHKKRIAEYKTLLKV |
| AvrPtoB | SEQ ID | MAGINGAGPSGAYFVGHTDPEPASGGAHGSSSGASSSNSPRLP |
| | NO.: 37 | APPDAPASQARDRREMLLRARPLSRQTREWVAQGMPPTAEA |
| | | GVPIRPQESAEAAAPQARAEERHTPEADAAASHVRTEGGRTP |
| | | QALAGTSPRHTGAVPHANRIVQQLVDAGADLAGINTMIDNA |
| | | MRRHAIALPSRTVQSILIEHFPHLLAGELISGSELATAFRAALR |
| | | REVRQQEASAPPRTAARSSVRTPERSTVPPTSTESSSGSNQRTL |
| | | LGRFAGLMTPNQRRPSSASNASASQRPVDRSPPRVNQVPTGA |
| | | NRVVMRNHGNNEADAALQGLAQQGVDMEDLRAALERHILH |
| | | RRPIPMDIAYALQGVGIAPSIDTGESLMENPLMNLSVALHRAL |
| | | GPRPARAQAPRPAVPVAPATVSRRPDSARATRLQVIPAREDYE |
| | | NNVAYGVRLLSLNPGAGVRETVAAFVNNRYERQAVVADIRA |
| | | ALNLSKQFNKLRTVSKADAASNKPGFKDLADHPDDATQCLF |
| | | GEELSLTSSVQQVIGLAGKATDMSESYSREANKDLVFMDMK |
| | | KLAQFLAGKPEHPMTRETLNAENIAKYAFRIVP |
| SdbA | SEQ ID | MHKKYNYYSLEKEKKTFWQHILDILKAPFRLPGWVVSFFLAR |
| | NO.: 38 | NITHVALNPNNIPQQRLIHLTKTSNRPEDDIVVINFKKRPPHKW |
| | | FNDTLIKIANTIAALPFVTPRLRTRLHYDNENDINHVNKLLAEI |
| | | DALVQGKSKQKYCKGRAFDWSKIHLKGLEFLDPKMRGYVYE |
| | | QLHEKYGYVSYTTKRKPNIEFFTLKTPDGSELDSVQVTGEDEE |
| | | KKPMGERKFIITCIARDQNFINWIKDLNYTAKNLGATAISFNY |
| | | RGVDYSRGLVWTENNLVDDILAQVQRLISLGADPKNICLDGM |
| | | CIGGAVATIAAAKLHEKGMKVKLNNERSFTSLSSLVFGFIVPE |
| | | LQTANWWSPLTYGRFLLAGVVYALLTPLIWLAGWPVDVTKA |
| | | WNRIPAQDKMYSVVRDKDNGLYDGVIHDHFCSIASLVDSQIN |
| | | SILYKLSTDQPLTEEEKQILCDDQFSHHFKPSQSVLKNPKYKGP |
| | | HFISRQDLVAELGHREEYTNHDYFLDRLREKFQLDRATRPVA |
| | | LAEDGEKDIDGISSQLSNNKERPLIIASSGGTGHISATHGIINDL |
| | | QSKTDNVVITQHHAELYKNKPFSITSVLIRIGVWFTSLPILEDIL |
| | | KGVMRFIGYPVLPSSSIFWDQMSKIQQSETKKENGIETGRTRP |
| | | YVDMLLDIYPEGYEYTAFNNATHLTSSIEDIQTMISFKGHVEE |
| | | DNRNIVYQNILQRLMHAAKQNTPYTRLISTQALSLGAICDAV |
| | | KYYNTVFLPVYNAERGTSYQPIAIDQYMTDLPSLGCIHFMNN |
| | | LEELTSEQRQLMEIHAVNMSEPFKEAHFGKEQGFKAVHNIDP |
| | | RNNPMIRNAFKDPSLTKYLDKTQSFDLHFNVYKKEKQNALPV |
| | | LNGKEKITIKPHAKIASIMIGSLAANASADYAKYLLNQGYEHIF |
| | | LFGGLNDSIAARIDQIINSYPAPTRDEIRKKIILLGNQSDVEMAP |
| | | IMTRSNCVVIRGGGLSVMEQMAMPIMDDKIVLLHHEDNEEGP |
| | | LTSGLSWEDGNSDKLIEYLSEKGAYAKKTSPGLCSGHLHEAE |
| | | KSFEKKYHGQLKSTETKKKVDLTIPQQETYSLKKEWDRKTGY |
| | | TESGHILSHQHRFFNTIPEVREPFCSKEDLHHNELSSQSLVSVS |
| | | AG |
| SidG | SEQ ID | MSRSKDEVLEANDSLFGITVQTWGTNDRPSNGMMNFADQQF |
| | NO.: 39 | FGGDVGHASINMKLPVTDKTKQWIEKYCYSQTYDQFKKVKG |
| | | NEDKTYEEYLKTAKRLIPVELKTQVTRKAQYDSNGNLVTTHE |
| | | KAYEQIYFDIDWSWWPGRLQNTEDDMVWEREGKHFEYDEK |
| | | WKEYLQPEQRVHRGKLGSRKMDYAPTSIIHQRDIPTSELEKIT |
| | | RDHKIHTIEEKLNVVKLLQSKIDEMPHTKMSPSMELMFKNLGI |
| | | NVEKLLDETKDNGVDPTNLEAMREYLTNRLTERKLELETELS |
| | | EAKKEVDSTQVKNKVEDVYYDFEYKLNQVRKKMEEVNSQL |
| | | EKMDSLLHKLEGNTSGPIPYTAEIDELMSVLPFLKEELELENG |
| | | TLSPKSIENLIDHIDELKNELASKQEKKNERNLNLIKKYEELCE |
| | | QYKDDEEGLEEALWEEGIDVEEVNSAKKDISKPAPEIQKLTDL |
| | | QEQLRNHKESGVKLSSELEETLNSSVKMWKTKIDSPCQVISES |
| | | SVKALVSKINSTRPELVKEKEQLPEQEESLSKEAKKAQEELIKI |
| | | QEFSQFYSENSSAYMVIGLPPHHQVSLPLAVNGKRGLHPEAM |
| | | LKKMHELVAGPEKKEFNLHTNNCSLTSIEVLSAGAQHDPLLH |
| | | SIMGTRALGFFGTPQQVLENAKLTSKTINEGKKSNIFTPLVTAS |
| | | PLDRALGYAMSIYMDPEASKAKQNAGLALGVLVGLAKTPGII |
| | | IGSLLNPKQGFNDILNTLNLVYSRNSTGLKVGLTLMALPAMIV |
| | | LAPLAAIQKGVEVIAETIAKPFKLIANLFKQKPESTDEITVSVGS |
| | | KKVAEKEGSYSNTALAGLVNSKIKSKIDENTITVEFQKSPQKM |
| | | IEEFESQLKENPGKVVVLSEKAHNAVLKFVSKSDDEALKQKF |
| | | YDCCNQSVARSQKFAPKTRDEIDELVEEVTSTDKTELTTSPRQ |
| | | EPSMSSTIDEEENIDSEHQIETGTESTMRI |
| VpdA | SEQ ID | MKTKQEVSQQDKLKDSKSSTPLQTKETWFISDALNITFDPYDF |
| | NO.: 40 | SISVTEQAPMPYRIVFSGGGSRILAHIGALDELTRHGLKFTEFS |
| | | GSSAGAMVAAFAYLGYNCSEIKQIISWFNEDKLLDSPLIFNFN |
| | | NIKQIFNKGGLSSAKLMRQAANYVILKKVMDIISDEKFKTRFA |
| | | KFQNFLEENIYRCPENITFQTLARIKEICPECELGEKLFITGTNL |
| | | STQKHEVFSIDTTPSMALADAIIISANLPIAFERICYQGNVYSDG |
| | | GISNNLPAHCFSEKGHKTTFLKHKDDVDFSVLALQFDNGLEE |
| | | NALYSQNPIPKWSWLSNTFYSLITGHPNVTENWYEDLQILRRH |
| | | AHQSILIKTPTIALTNLTISQDTKKALVESGRTAAKTYLELHEF |
| | | YTDDYGNIRHNECLHEKFQKPEELLDYCVLHSHFELLKKIKQ |
| | | AISCSQYLEKGYKHYLCELCDNLLPPQLKCPNEGSGTEQPEIK |
| | | LEKDTIICEKNNNSGLTFSMTFFGVPSPLVKTLNQDSPELKIKL |
| | | FTGLYPILIQNWQNLCPVSGISGILNSIRMSFVEISSTDTCIKTLI |
| | | DKLNEIEIGHFLIFVFKAALKNYDKHDFILLLKNLKHLHHSIELI |
| | | RNKPFHSDDRFYGQWSFEGHDPKRILEFIKSDDISGLMTILEDK |
| | | KALPNNKPN |
| Lpg0969 | SEQ ID | MVSLEHIQKLISECRKLGKDGLDNGTNGLIPELEIDVVPPSAFL |
| | NO.: 41 | GVGNNPAIFVNSKTYKLMRTTHEKWVENKTIVFKSYLLSQPA |
| | | IKIIGAIVHETGHAFNVAAKIPNTEANACIFEIEVLMRLFQVKSP |
| | | LLLGCTELDMQSYFKSRLTDYNKCVKDCQCLAEMVEFITHQF |
| | | KLDEVSISEKENQIPLLSISNKWPGLFAKKQIAPDMDKLLTSPV |
| | | TITPEVKILFYQLVKEHFHSPETEIKLDI |
| Lpg1978 | SEQ ID | MYKIYSYLGWRIDMKTENLPQAGQEAQIDKKIHFIWVGHIMP |
| | NO.: 42 | QKNIQVVSEWAEKNPGYETIIWVDKKIAPAKELDLFILDMKSK |
| | | GITVKDINEEGVCRDSIRHELDQESPNYGMVSDMLRLNILAAE |
| | | GGIYLDSDILCSAPFPDEIYAPFGFLLSPWSQGANNTLCNDIILC |
| | | SKGNQIIQQLADAIEQSYIARDSFEFTHEYASMKETKGERIAKT |
| | | LGVTGPGFLFHQLKKMGILNDKSEMEAIHWELQDQRYLIDGS |
| | | VKEPDYFYVPQNNTNDASWVPSIKRPGIENMSFQERLENAVQ |
| | | LIAFDIQKTGLFNLDHYANELKVKQNSWCIAAETSPELKPDSY |
| | | LLIRPRDKTGEWTLYYVDEDKKLNPVTLPVIKGAIKLSEVSDP |
| | | LRKFHTLLSQVSDPVNPTAHELKQIGRALIELK |
| | | PRQDEWHCKNKWSGAEEIAQELWQRITSNETLRAQIKQCFTQ |
| | | FESLKPRVAELGLTRASGAGTEVEAHESTVKEQEIISQNTVGE |
| | | EGTKEKNSVQLASENSSDEKIKTAHDLIDEIIQDVIQLDGKLGL |
| | | LGGNTRQLEDGRVINIPNGAAMIFDDYKKYKQGELTAESALE |
| | | SMIKIAKLSNQLNRHTFFNQRQPETGQFYKKVAAIDLQTTIAA |
| | | EYDNNHGLRI |
| YopE | SEQ ID | MKISSFISTSLPLPTSVSGSSSVGEMSGRSVSQQTSDQYANNLA |
| | NO.: 43 | GRTESPQGSSLASRIIERLSSVAHSVIGFIQRMFSEGSHKPVVTP |
| | | APTPAQMPSPTSFSDSIKQLAAETLPKYMQQLNSLDAEMLQK |
| | | NHDQFATGSGPLRGSITQCQGLMQFCGGELQAEASAILNTPV |
| | | CGIPFSQWGTIGGAASAYVASGVDLTQAANEIKGLAQQMQKL |
| | | LSLM |
| SptP | SEQ ID | MLKYEERKLNNLTLSSFSKVGVSNDARLYIAKENTDKAYVAP |
| | NO.: 44 | EKFSSKVLTWLGKMPLFKNTEVVQKHTENIRVQDQKILQTFL |
| | | HALTEKYGETAVNDALLMSRINMNKPLTQRLAVQITECVKA |
| | | ADEGFINLIKSKDNVGVRNAALVIKGGDTKVAEKNNDVGAES |
| | | KQPLLDIALKGLKRTLPQLEQMDGNSLRENFQEMASGNGPLR |
| | | SLMTNLQNLNKIPEAKQLNDYVTTLTNIQVGVARFSQWGTCG |
| | | GEVERWVDKASTHELTQAVKKIHVIAKELKNVTAELEKIEAG |
| | | APMPQTMSGPTLGLARFAVSSIPINQQTQVKLSDGMPVPVNTL |
| | | TFDGKPVALAGSYPKNTPDALEAHMKMLLEKECSCLVVLTSE |
| | | DQMQAKQLPPYFRGSYTFGEVHTNSQKVSSASQGEAIDQYN |
| | | MQLSCGEKRYTIPVLHVKNWPDHQPLPSTDQLEYLADRVKNS |
| | | NQNGAPGRSSSDKHLPMIHCLGGVGRTGTMAAALVLKDNPH |
| | | SNLEQVRADFRDSRNNRMLEDASQFVQLKAMQAQLLMTTAS |
| SopE2 | SEQ ID | MTNITLSTQHYRIHRSDVEPVKEKTTEKDIFAKSITAVRNSFISL |
| | NO.: 45 | STSLSDRFSLHQQTDIPTTHFHRGNASEGRAVLTSKTVKDFML |
| | | QKLNSLDIKGNASKDPAYARQTCEAILSAVYSNNKDQCCKLL |
| | | ISKGVSITPFLKEIGEAAQNAGLPGEIKNGVFTPGGAGANPFVV |
| | | PLIASASIKYPHMFINHNQQVSFKAYAEKIVMKEVTPLFNKGT |
| | | MPTPQQFQLTIENIANKYLQNAS |
| SopB/SigD | SEQ ID | MQIQSFYHSASLKTQEAFKSLQKTLYNGMQILSGQGKAPAKA |
| | NO.: 46 | PDARPEIIVLREPGATWGNYLQHQKASNHSLHNLYNLQRDLL |
| | | TVAATVLGKQDPVLTSMANQMELAKVKADRPATKQEEAAA |
| | | KALKKNLIELIAARTQQQDGLPAKEAHRFAAVAFRDAQVKQL |
| | | NNQPWQTIKNTLTHNGHHYTNTQLPAAEMKIGAKDIFPSAYE |
| | | GKGVCSWDTKNIHHANNLWMSTVSVHEDGKDKTLFCGIRHG |
| | | VLSPYHEKDPLLRHVGAENKAKEVLTAALFSKPELLNKALAG |
| | | EAVSLKLVSVGLLTASNIFGKEGTMVEDQMRAWQSLTQPGK |
| | | MIHLKIRNKDGDLQTVKIKPDVAAFNVGVNELALKLGFGLKA |
| | | SDSYNAEALHQLLGNDLRPEARPGGWVGEWLAQYPDNYEV |
| | | VNTLARQIKDIWKNNQHHKDGGEPYKLAQRLAMLAHEIDAV |
| | | PAWNCKSGKDRTGMMDSEIKREIISLHQTHMLSAPGSLPDSG |
| | | GQKIFQKVLLNSGNLEIQKQNTGGAGNKVMKNLSPEVLNLSY |
| | | QKRVGDENIWQSVKGISSLITS |
| SipA | SEQ ID | MVTSVRTQPPVIMPGMQTEIKTQATNLAANLSAVRESATTTL |
| | NO.: 47 | SGEIKGPQLEDFPALIKQASLDALFKCGKDAEALKEVFTNSNN |
| | | VAGKKAIMEFAGLFRSALNATSDSPEAKTLLMKVGAEYTAQI |
| | | IKDGLKEKSAFGPWLPETKKAEAKLENLEKQLLDIIKNNTGGE |
| | | LSKLSTNLVMQEVMPYIASCIEHNFGCTLDPLTRSNLTHLVDK |
| | | AAAKAVEALDMCHQKLTQEQGTSVGREARHLEMQTLIPLLL |
| | | RNVFAQIPADKLPDPKIPEPAAGPVPDGGKKAEPTGINININIDS |
| | | SNHSVDNSKHINNSRSHVDNSQRHIDNSNHDNSRKTIDNSRTF |
| | | IDNSQRNGESHHSTNSSNVSHSHSRVDSTTHQTETAHSASTGA |
| | | IDHGIAGKIDVTAHATAEAVTNASSESKDGKVVTSEKGTTGET |
| | | TSFDEVDGVTSKSIIGKPVQATVHGVDDNKQQSQTAEIVNVK |
| | | PLASQLAGVENVKTDTLQSDTTVITGNKAGTTDNDNSQTDKT |
| | | GPFSGLKFKQNSFLSTVPSVTNMHSMHFDARETFLGVIRKALE |
| | | PDTSTPFPVRRAFDGLRAEILPNDTIKSAALKAQCSDIDKHPEL |
| | | KAKMETLKEVITHHPQKEKLAEIALQFAREAGLTRLKGETDY |
| | | VLSNVLDGLIGDGSWRAGPAYESYLNKPGVDRVITTVDGLH |
| | | MQR |
| YpkA | SEQ ID | MKSVKIMGTMPPSISLAKAHERISQHWQNPVGELNIGGKRYRI |
| | NO.: 48 | IDNQVLRLNPHSGFSLFREGVGKIFSGKMFNFSIARNLTDTLH |
| | | AAQKTTSQELRSDIPNALSNLFGAKPQTELPLGWKGEPLSGAP |
| | | DLEGMRVAETDKFAEGESHISIIETKDKQRLVAKIERSIAEGHL |
| | | FAELEAYKHIYKTAGKHPNLANVHGMAVVPYGNRKEEALLM |
| | | DEVDGWRCSDTLRTLADSWKQGKINSEAYWGTIKFIAHRLLD |
| | | VTNHLAKAGVVHNDIKPGNVVFDRASGEPVVIDLGLHSRSGE |
| | | QPKGFTESFKAPELGVGNLGASEKSDVFLVVSTLLHCIEGFEK |
| | | NPEIKPNQGLRFITSEPAHVMDENGYPIHRPGIAGVETAYTRFI |
| | | TDILGVSADSRPDSNEARLHEFLSDGTIDEESAKQILKDTLTGE |
| | | MSPLSTDVRRITPKKLRELSDLLRTHLSSAATKQLDMGGVLSD |
| | | LDTMLVALDKAEREGGVDKDQLKSFNSLILKTYRVIEDYVKG |
| | | REGDTKNSSTEVSPYHRSNFMLSIVEPSLQRIQKHLDQTHSFSD |
| | | IGSLVRAHKHLETLLEVLVTLSQQGQPVSSETYGFLNRLAEAK |
| | | ITLSQQLNTLQQQQESAKAQLSILINRSGSWADVARQSLQRFD |
| | | STRPVVKFGTEQYTAIHRQMMAAHAAITLQEVSEFTDDMRNF |
| | | TVDSIPLLIQLGRSSLMDEHLVEQREKLRELTTIAERLNRLERE |
| | | WM |
| YopM | SEQ ID | MFINPRNVSNTFLQEPLRHSSNLTEMPVEAENVKSKTEYYNA |
| | NO.: 49 | WSEWERNAPPGNGEQREMAVSRLRDCLDRQAHELELNNLGL |
| | | SSLPELPPHLESLVASCNSLTELPELPQSLKSLQVENNNLKALP |
| | | DLPPSLKKLHVRENDLTDLPELPQSLESLRVDNNNLKALSDLP |
| | | PSLEYLTASSNKLEELPELQNLPFLAAIYADNNLLETLPDLPPS |
| | | LKKLHVRENDLTDLPELPQSLESLQVDNNNLKALSDLPPSLEY |
| | | LTASSNKLEELPELQNLPFLAAIYADNNLLETLPDLPPHLEILV |
| | | ASYNSLTELPELPQSLKSLRVDNNNLKALSDLPPSLEYLTASS |
| | | NKLEELPELQNLPFLAAIYADNNLLETLPDLPPSLKKLHVREN |
| | | DLTDLPELPQSLTFLDVSDNNISGLSELPPNLYYLDASSNEIRSL |
| | | CDLPPSLVDLNVKSNQLSELPALPPHLERLIASFNYLAEVPELP |
| | | QNLKQLHVEQNALREFPDIPESLEELEMDSERVVDPYEFAHET |
| | | TDKLEDDVFE |
| Amatoxin | SEQ ID | MSDINATRLPIWGIGCNPCVGDDVTTLLTRGEALC |
| | NO.: 50 | |
| Phallacidin | SEQ ID | MSDINATRLPAWLVDCPCVGDDVNRLLTRGESLC |
| | NO.: 51 | |
| Killer | SEQ ID | MIKPERSILTILIGILCLLAYVLANGEPHDGDNEWSSYCSDQGF |
| toxin | NO.: 52 | RRSDDGLVTTPDVGQESIGKNSINGSELVDYLQCLKVRLNGQ |
| KP1 | | KQVVSNDGWLLLLVQEPSVNVTQKAMSECNYNVSSGHKAGS |
| | | YIQVTNTPADYKVISRRGSYEGDQLPEDVKPYFGVQKTSDYR |
| | | PISKRINPNLTLRQLAYNFAALNMCSLWCNSCISRSCPYYIAEL |
| | | TVHVNNIHHGTVWLHHFCRNASPQGGNLYSTLTISHKDTAYY |
| | | VGTGWWKVRSTAATTNDVAGDWYPASWNQYWCGPHY |
| Killer | SEQ ID | MLIFSVLMYLGLLLAGASALPNGLSPRNNAFCAGFGLSCKWE |
| toxin | NO.: 53 | CWCTAHGTGNELRYATAAGCGDHLSKSYYDARAGHCLFSD |
| KP6 | | DLRNQFYSHCSSLNNNMSCRSLSKRTIQDSATDTVDLGAELH |
| | | RDDPPPTASDIGKRGKRPRPVMCQCVDTTNGGVRLDAVTRA |
| | | ACSIDSFIDGYYTEKDGFCRAKYSWDLFTSGQFYQACLRYSH |
| | | AGTNCQPDPQYE |
| Killer | SEQ ID | MTKPTQVLVRSVSILFFITLLHLVVALNDVAGPAETAPVSLLP |
| Toxin K1 | NO.: 54 | REAPWYDKIWEVKDWLLQRATDGNWGKSITWGSFVASDAG |
| | | VVIFGINVCKNCVGERKDDISTDCGKQTLALLVSIFVAVTSGH |
| | | HLIWGGNRPVSQSDPNGATVARRDISTVADGDIPLDFSALNDI |
| | | LNEHGISILPANASQYVKRSDTAEHTTSFVVTNNYTSLHTDLI |
| | | HHGNGTYTTFTTPHIPAVAKRYVYPMCEHGIKASYCMALND |
| | | AMVSANGNLYGLAEKLFSEDEGQWETNYYKLYWSTGQWIM |
| | | SMKFIEESIDNANNDFEGCDTGH |
| Killer | SEQ ID | MGHLAILFSIIAVLNIATAVASSDSIYLKGHRVGQDIDSLYRVY |
| Toxin | NO.: 55 | DNGTMYPVTFNEWLNDLTGMNDLATNNATILKRDSSDVSCV |
| K28 | | TETCQYVDYHVDDEGVITIDISTYRIPVEWDSGSAGNASYGVS |
| (KHR) | | KRDTKYETFCKKKICGINVSGFCNAYDFAVHAFDFGGSVYNP |
| | | VSGITDRIKEATKRDKTECLGYELDHVRIDPAVDWSISISTWK |
| | | QGSANCDTQASADSLKCAAQKALESEHNHQKTAFCIHLDNG |
| | | GSFNLDIRLISELSFSKYNPWALPCPKYKGSNSWQVVSDCFQ |
| Killer | SEQ ID | MPRFAIIFALLIAYSLFLSTLFTGSIPDRANTVTSNAPCQVVIWD |
| Toxin | NO.: 56 | WIRTRRICNCCSRLCYSLLGRSNLSRTAKRGVCTIAGAVLATA |
| K28 | | AVIVAAVLVGKSSGSATKRGLTKTISVLNHTIPFTDHILNGQTL |
| (KHS) | | SNGTGSNFVTIGFSGYAVHATIKRASTTDIISWVIPESMEPTLA |
| | | RVASYVSSSSINLAAVPDTGGNASALSFQNAVQEFATSWVSM |
| | | TYDQSYGDLRNVANDEGGEEILILMRKRSYRISFQVIETGSTA |
| | | LLLRTRRVVSQLITMTYLVTVQARVGIQIGDIFQHYGCrIDNYV |
| | | MTSISVLRTLEDKAFHENKLLIVREPPNKSNQDANQSYRLRPF |
| | | SANDLIQNLKSVDIGFLAFCSFFDKYAHYPEIIMMKITIFISKGN |
| | | LWSIIYVIQARYVRKRVMKVRGQMPGGLLTNMESLLNIVSTP |
| | | NLNISEFHIQTHSMSQSKPMYFQKQCYSSQNNIIYIYNSIHITCG |
| | | AVYVIVHDVRTPSVFVLIELRNCKPLKNSWCETTKTSPRDTKI |
| | | KKNEYNETVCRRAGALLDGRVRTIRFLMMRTHWSRVKGVSC |
| | | NTANRLSRFCNHVVSYYPSQNATIHLLPTSLRAESLEQQYTTR |
| | | PLSSSNNRFCCLKSIFINNCKKACESPSLVSCNLQQTAELLMVY |
| | | YLYICEACYVSRNHDLLSKQCMSTVRAVYVARMRLPKFRSTF |
| | | PCMPRLCWLVNGVVVV |
| Anthrax | SEQ ID | MHVKEKEKNKDENKRKDEERNKTQEEHLKEIMKHIVKIEVK |
| lethal | NO.: 57 | GEEAVKKEAAEKLLEKVPSDVLEMYKAIGGKIYIVDGDITKHI |
| factor | | SLEALSEDKKKIKDIYGKDALLHEHYVYAKEGYEPVLVIQSSE |
| endopeptidase | | DYVENTEKALNVYYEIGKILSRDILSKINQPYQKFLDVLNTIKN |
| | | ASDSDGQDLLFTNQLKEHPTDFSVEFLEQNSNEVQEVFAKAF |
| | | AYYIEPQHRDVLQLYAPEAFNYMDKFNEQEINLSLEELKDQR |
| | | MLSRYEKWEKIKQHYQHWSDSLSEEGRGLLKKLQIPIEPKKD |
| | | DIIHSLSQEEKELLKRIQIDSSDFLSTEEKEFLKKLQIDIRDSLSE |
| | | EEKELLNRIQVDSSNPLSEKEKEFLKKLKLDIQPYDINQRLQDT |
| | | GGLIDSPSINLDVRKQYKRDIQNIDALLHQSIGSTLYNKIYLYE |
| | | NMNINNLTATLGADLVDSTDNTKINRGIFNEFKKNFKYSISSN |
| | | YMIVDINERPALDNERLKWRIQLSPDTRAGYLENGKLILQRNI |
| | | GLEIKDVQIIKQSEKEYIRIDAKVVPKSKIDTKIQEAQLNINQE |
| | | WNKALGLPKYTKLITFNVHNRYASNIVESAYLILNEWKNNIQ |
| | | SDLIKKVTNYLVDGNGRFVFTDITLPNIAEQYTHQDEIYEQVH |
| | | SKGLYVPESRSILLHGPSKGVELRNDSEGFIHEFGHAVDDYAG |
| | | YLLDKNQSDLVTNSKKFIDIFKEEGSNLTSYGRTNEAEFFAEA |
| | | FRLMHSTDHAERLKVQKNAPKTFQFINDQIKFIINS |
| Shiga | SEQ ID | MKCILLKWVLCLLLGFSSVSYSREFTIDFSTQQSYVSSLNSIRT |
| Toxin | NO.: 58 | EISTPLEHISQGTTSVSVINHTPPGSYFAVDIRGLDVYQARFDH |
| | | LRLIIEQNNLYVAGFVNTATNTFYRFSDFAHISVPGVTTVSMT |
| | | TDSSYTTLQRVAALERSGMQISRHSLVSSYLALMEFSGNTMT |
| | | RDASRAVLRFVTVTAEALRFRQIQREFRQALSETAPVYTMTP |
| | | GDVDLTLNWGRISNVLPEYRGEDGVRVGRISFNNISAILGTVA |
| | | VILNCHHQGARSVRAVNEESQPECQITGDRPVIKINNTLWESN |
| | | TAAAFLNRKSQSLYTTGE |
| Ricin | SEQ ID | MYAVATWLCFGSTSGWSFTLEDNNIFPKQYPIINFTTAGATVQ |
| Toxin | NO.: 59 | SYTNFIRAVRGRLTTGADVRHDIPVLPNRVGLPINQRFILVELS |
| | | NHAELSVTLALDVTNAYVVGYRAGNSAYFFHPDNQEDAEAI |
| | | THLFTDVQNRYTFAFGGNYDRLEQLAGNLRENIELGNGPLEE |
| | | AISALYYYSTGGTQLPTLARSFIICIQMISEAARFQYIEGEMRTR |
| | | IRYNRRSAPDPSVITLENSWGRLSTAIQESNQGAFASPIQLQRR |
| | | NGSKFSVYDVSILIPIIALMVYRCAPPPSSQFSLLIRPVVPNFNA |
| | | DVCMDPEPIVRIVGRNGLCVDVRDGRFHNGNAIQLWPCKSNT |
| | | DANQLWTLKRDNTIRSNGKCLTTYGYSPGVYVMIYDCNTAA |
| | | TDATRWQIWDNGTIINPRSSLVLAATSGNSGTTLTVQTNIYAV |
| | | SQGWLPTNNTQPFVTTIVGLYGLCLQANSGQVWIEDCSSEKA |
| | | EQQWALYADGSIRPQQNRDNCLTSDSNIRETVVKILSCGPASS |
| | | GQRWMFKNDGTILNLYSGLVLDVRRSDPSLKQIILYPLHGDP |
| | | NQIWLPLF |

A cytotoxic reporter of the invention can be, for example, a ribosomally-encoded xenobiotic agent, a ribosomally-encoded poison, a ribosomally-encoded endogenous or exogenous gene that results in severe growth defects upon mild overexpression, a ribosomally-encoded recombinase that excises an essential gene for viability, or a limiting factor (or multiple factors) that can be involved in the synthesis of a toxic secondary metabolite.

The system of the invention can use the reconstitution of a transcription factor mediated by the interaction between a protein fused to an AD, for example, Dof1, and another protein fused to a DBD, for example, TetR.

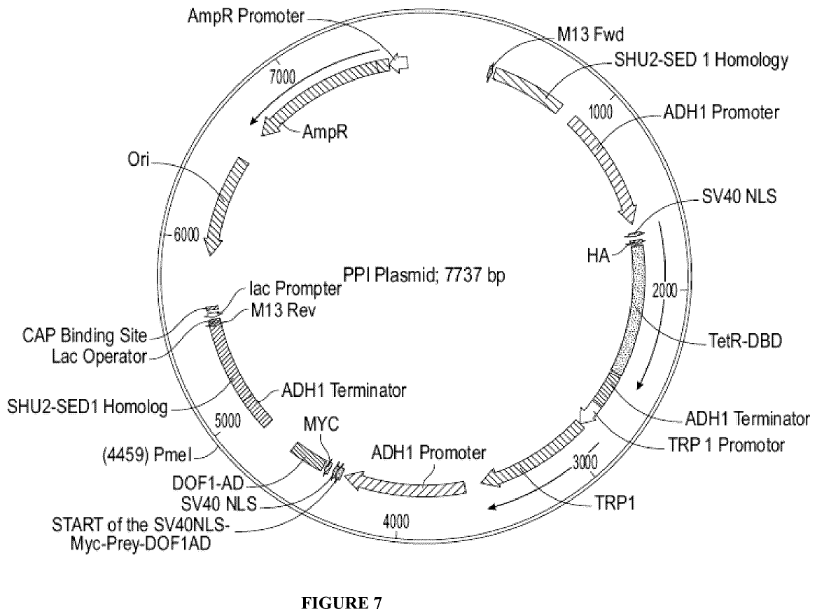

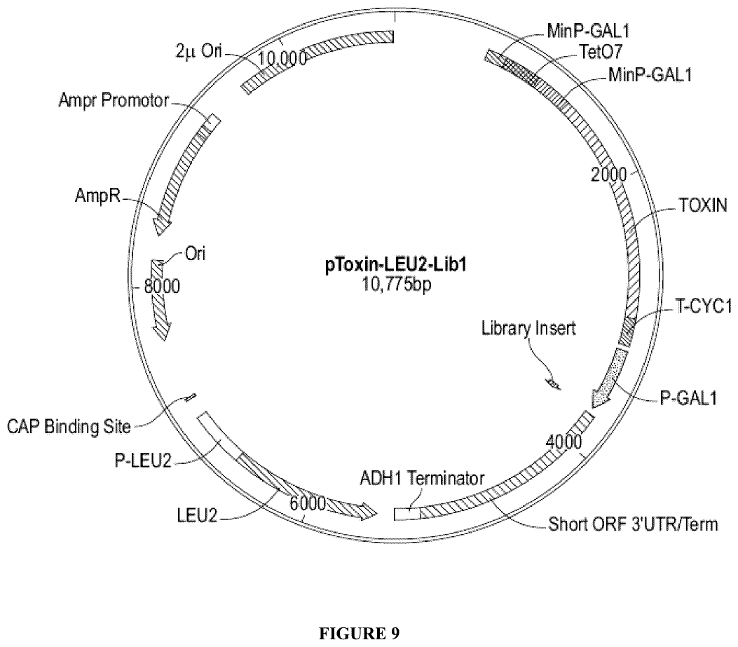

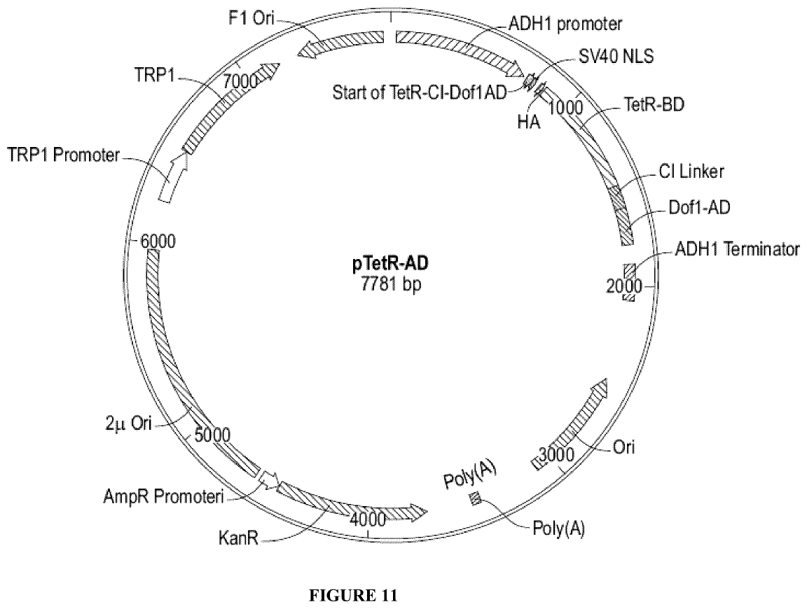

To identify peptides that can disrupt a PPI, a PPI integration plasmid (Plasmid 1; FIG. 7), a selection and library plasmid (Plasmid 2; FIG. 9), and a confirmation (Plasmid 3; FIG. 11) can be used.

Plasmid 1 can contain, for example, two restriction sites that enable the integration of two proteins that constitute the PPI of interest. The PPI of interest can involve a pair of domains having known importance for carcinogesis, such as p53-MDM2, RAS-RASRBD, and MYC-MAX. The PPI of interest can also involve the interaction of an oncogene (such as Cdk1, Cdk2, Cdk3, Cdk4, Cdk6, c-MYC, EGFR, HER2, K-Ras, PDGFR, Raf kinase, and VEGF) or a tumor suppressor (such as BRCA1, BRCA2, cyclin-dependent kinase inhibitor 1C, PTEN, p16, p27, p53, p73, and Retinoblastoma protein (pRb)) with a known cellular interaction partner. The PPI of interest can involve the interaction of a protein involved in the DNA repair pathway (such as ATM, ATRX, BRCA1, BRCA2, ERCC1, FANCB, FANCF, FEN1, HMGA1, HMGA1, MDC1, MGMT, MLH1, MSH2, MSH4, Mre11A, NBS1, NEIL1, PARP1, PARP2, PMS2, RAD51, RAD52, RAD54, RAD51AP1, WRN, and XPF) with another cellular factor.

When designating a DNA sequence, an N means any possible deoxynucleotide.

Plasmid 1 can encode for the fusion of an AD (such as Dof1) or another gene activating moiety and a DBD (such as TetR) to each protein driven by either a strong promoter and terminator (such as ADH1), or by an inducible promoter (such as GAL1). Other exemplary activation domains include those of VP16 and B42AD. Other exemplary DBDs include those of GAL4 or LexA. Each protein fusion can be tagged for subsequent biochemical experiments with, for example, a FLAG, HA, or His tag. Plasmid 1 can also include bacterial selection and propagation markers (i.e. ori and AmpR), and yeast replication and selection markers (i.e. TRP1 and CEN or 2 um). The plasmid can also be integrated into the genome at a specified locus. The sequence of Plasmid 1 can be:

(SEQ ID NO.: 63)
TCGCGCGTTTCGGTGATGACGGTGAAAACCTCTGACACATGCAGCTCCCGGAGACGGTCACAGCTTGTCTGTAAGCGGATGCC
GGGAGCAGACAAGCCCGTCAGGGCGCGTCAGCGGGTGTTGGCGGGTGTCGGGGCTGGCTTAACTATGCGGCATCAGAGCAGAT
TGTACTGAGAGTGCACCATATGCGGTGTGAAATACCGCACAGATGCGTAAGGAGAAAATACCGCATCAGGCGCCATTCGCCAT
TCAGGCTGCGCAACTGTTGGGAAGGGCGATCGGTGCGGGCCTCTTCGCTATTACGCCAGCTGGCGAAAGGGGGATGTGCTGCA
AGGCGATTAAGTTGGGTAACGCCAGGGTTTTCCCAGTCACGACGTTGTAAAACGACGGCCAGTGAATTCGAGCTCGGTACCCG
TCTTCTGTCCCAGTTACCGAATCTAAGGGCACTACCACCAAAGAAACAGGTGTTACTACCAAACAAACCACAGCCAACCCAAG
TCTAACCGTCTCCACAGTCGTCCCAGTTTCATCCTCTGCTTCTTCTCATTCCGTTGTCATCAACAGTAACGGTGCTAACGTCG
TCGTTCCAGGTGCTTTAGGTTTGGCTGGTGTTGCTATGTTATTCTTATAAACGGTGGTGTTTGACACATCCGCCTTCTTAATG
CTTTCTTTCAGTATTATGTTATTTTTTTGTTATTCGTTTTTCACTTCTAGGCTTTTTGACAGACTAGCCCCGTTATACCACCA
TCTTTGTGGGAAAGCCCCTAAATTGCCCTGAGCAGTATCGTTTCATGTCTAGTCTCTTTAAAGATGTTTCTTACACTTCTCCT
ATGCACATATATTAATTAAAGTCCAATGCTAGTAGAGAAGGGGGGTAACACCCCTCCGCGCTCTTTTCCGATTTTTTTCTAAA
CCGTGGAATATTTCGGATATCCTTTTGTTGTTTCCGGGTGTACAATATGGACTTCCTCTTTTCTGGCAACCAAACCCATACAT
CGGGATTCCTATAATACCTTCGTTGGTCTCCCTAACATGTAGGTGGCGGAGGGGAGATATACAATAGAACAGATACCAGACAA
GACATAATGGGCTAAACAAGACTACACCAATTACACTGCCTCATTGATGGTGGTACATAACGAACTAATACTGTAGCCCTAGA
CTTGATAGCCATCATCATATCGAAGTTTCACTACCCTTTTTCCATTTGCCATCTATTGAAGTAATAATAGGCGCATGCAACTT
CTTTTCTTTTTTTTTCTTTTCTCTCTCCCCCGTTGTTGTCTCACCATATCCGCAATGACAAAAAAATGATGGAAGACACTAAA
GGAAAAAATTAACGACAAAGACAGCACCAACAGATGTCGTTGTTCCAGAGCTGATGAGGGGTATCTCGAAGCACACGAAACTT
TTTCCTTCCTTCATTCACGCACACTACTCTCTAATGAGCAACGGTATACGGCCTTCCTTCCAGTTACTTGAATTTGAAATAAA
AAAAAGTTTGCTGTCTTGCTATCAAGTATAAATAGACCTGCAATTATTAATCTTTTGTTTCCTCGTCATTGTTCTCGTTCCCT
TTCTTCCTTGTTTCTTTTTCTGCACAATATTTCAAGCTATACCAAGCATACAATCAACTCCAAGCTTTGCAAAGATGGGGTCA
AAGGCCGAGCTAATCCCAGAGCCCCCTAAAAAAAAGAGAAAGGTCGAGCTGGGAACTGCGGCAGAGTACCCGTATGATGTACC
GGACTATGCCGGAGGTATGTCTAGATTGGACAAGTCTAAGGTTATCAACTCTGCTTTGGAATTGTTGAACGAAGTTGGTATCG
AAGGTTTGACTACTAGAAAGTTGGCTCAAAAGTTGGGTGTTGAACAACCAACTTTGTACTGGCACGTTAAGAACAAGAGAGCT
TTGTTGGACGCTTTGGCTATCGAAATGTTGGACAGACACCACACTCACTTCTGTCCATTGGAAGGTGAATCTTGGCAAGACTT
CTTGAGAAACAACGCTAAGTCTTTCAGATGTGCTTTGTTGTCTCACAGAGACGGTGCTAAGGTTCACTTGGGTACTAGACCAA
CTGAAAAGCAATACGAAACTTTGGAAAACCAATTGGCTTTCTTGTGTCAACAAGGTTTCTCTTTGGAAAACGCTTTGTACGCT
TTGTCTGCTGTTGGTCACTTCACTTTGGGTTGTGTTTTGGAAGACCAAGAACACCAAGTTGCTAAGGAAGAAAGAGAAACTCC
AACTACTGACTCTATGCCACCATTGTTGAGACAAGCTATCGAATTGTTCGACCACCAAGGTGCTGAACCAGCTTTCTTGTTCG
GTTTGGAATTGATCATCTGTGGTTTGGAAAAGCAATTGAAGTGTGAATCTGGTTCTGGGCAACCATCTTTGAGATCTGAATAC
GAATACCCAGTTTTCTCTCACGTTCAAGCTGGTATGTTCTCTCCAGAATTGAGAACTTTCACTAAGGGTGACGCTGAAAGATG
GGTTTCTGGTCCAGGCGCGCCACTTCTAAATAAGCGAATTTCTTATGATTTATGATTTTTATTATTAAATAAGTTATAAAAAA
AATAAGTGTATACAAATTTTAAAGTGACTCTTAGGTTTTAAAACGAAAATTCTTATTCTTGAGTAACTCTTTCCTGTAGGTCA
GGTTGCTTTCTCAGGTATAGCATGAGGTCGCTCTTATTGACCACACCTCTACCGGCAGATCTGTACAATCTTGATCCGGAGCT
TTTCTTTTTTTGCCGATTAAGAATTAATTCGGTCGAAAAAAGAAAAGGAGAGGGCCAAGAGGGAGGGCATTGGTGACTATTGA
GCACGTGAGTATACGTGATTAAGCACACAAAGGCAGCTTGGAGTATGTCTGTTATTAATTTCACAGGTAGTTCTGGTCCATTG
GTGAAAGTTTGCGGCTTGCAGAGCACAGAGGCCGCAGAATGTGCTCTAGATTCCGATGCTGACTTGCTGGGTATTATATGTGT
GCCCAATAGAAAGAGAACAATTGACCCGGTTATTGCAAGGAAAATTTCAAGTCTTGTAAAAGCATATAAAAATAGTTCAGGCA
CTCCGAAATACTTGGTTGGCGTGTTTCGTAATCAACCTAAGGAGGATGTTTTGGCTCTGGTCAATGATTACGGCATTGATATC
GTCCAACTGCATGGAGATGAGTCGTGGCAAGAATACCAAGAGTTCCTCGGTTTGCCAGTTATTAAAAGACTCGTATTTCCAAA
AGACTGCAACATACTACTCAGTGCAGCTTCACAGAAACCTCATTCGTTTATTCCCTTGTTTGATTCAGAAGCAGGTGGGACAG
GTGAACTTTTGGATTGGAACTCGATTTCTGACTGGGTTGGAAGGCAAGAGAGCCCCGAAAGCTTACATTTTATGTTAGCTGGT
GGACTGACGCCAGAAAATGTTGGTGATGCGCTTAGATTAAATGGCGTTATTGGTGTTGATGTAAGCGGAGGTGTGGAGACAAA
TGGTGTAAAAGACTCTAACAAAATAGCAAATTTCGTCAAAAATGCTAAGAAATAGGTTATTACTGAGTAGTATTTATTTAAGT
ATTGTTTGTGCACTTGCCTGCGGTGTGAAATACCGCACAGATGCGTAAGGAATATTTCGGATATCCTTTTGTTGTTTCCGGGT
GTACAATATGGACTTCCTCTTTTCTGGCAACCAAACCCATACATCGGGATTCCTATAATACCTTCGTTGGTCTCCCTAACATG
TAGGTGGCGGAGGGGAGATATACAATAGAACAGATACCAGACAAGACATAATGGGCTAAACAAGACTACACCAATTACACTGC
CTCATTGATGGTGGTACATAACGAACTAATACTGTAGCCCTAGACTTGATAGCCATCATCATATCGAAGTTTCACTACCCTTT
TTCCATTTGCCATCTATTGAAGTAATAATAGGCGCATGCAACTTCTTTTCTTTTTTTTTCTTTTCTCTCTCCCCCGTTGTTGT
CTCACCATATCCGCAATGACAAAAAAATGATGGAAGACACTAAAGGAAAAAATTAACGACAAAGACAGCACCAACAGATGTCG
TTGTTCCAGAGCTGATGAGGGGTATCTCGAAGCACACGAAACTTTTTCCTTCCTTCATTCACGCACACTACTCTCTAATGAGC
AACGGTATACGGCCTTCCTTCCAGTTACTTGAATTTGAAATAAAAAAAAGTTTGCTGTCTTGCTATCAAGTATAAATAGACCT
GCAATTATTAATCTTTTGTTTCCTCGTCATTGTTCTCGTTCCCTTTCTTCCTTGTTTCTTTTTCTGCACAATATTTCAAGCTA
TACCAAGCATACAATCAACTCCAAGCTTTGCAAAGATGGGGTCAAAGGCCGAGCTAATCCCAGAGCCCCCTAAAAAAAAGAGA
AAGGTCGAGCTGGGAACTGCGGCAGAGGAGCAGAAGCTGATCTCAGAGGAGGACCTGTTTAAACCAGGAGGCGGTTCTGGTCC
AGGTACTGAAGACGCTGAAGCTGTTGCTTTGGGTTTGGGTTTGTCTGACTTCCCATCTGCTGGTAAGGCTGTTTTGGACGACG
AAGACTCTTTCGTTTGGCCAGCTGCTTCTTTCGACATGGGTGCTTGTTGGGCTGGTGCTGGTTTCGCTGACCCAGACCCAGCT
TGTATCTTCTTGAACTTGCCATGAGCCCATCTTTTTTTTGGACCTAAATTCTTCATGAAAATATATTACGAGGGCTTATTCAG
AAGCTTTGGACTTCTTCGCCAGAGGTTTGGTCAAGTCTCCAATCAAGGTTGTCGGCTTGTCTACCTTGCCAGAAATTTACGAA
AAGATGGAAAAGGGTCAAATCGTTGGTAGATACGTTGTTGACACTTCTAAATAAGCGAATTTCTTATGATTTATGATTTTTAT
TATTAAATAAGTTATAAAAAAAATAAGTGTATACAAATTTTAAAGTGACTCTTAGGTTTTAAAACGAAAATTCTTATTCTTGA
GTAACTCTTTCCTGTAGGTCAGGTTGCTTTCTCAGGTATAGCATGAGGTCGCTCTTATTGACCACACCTCTACCGGCCGGTCG
AAATTCCCCTACCCTATGAACATATTCCATTTTGTAATTTCGTGTCGCGTTGCGTGTAAAACATCCTCTCATTCAAGACAGGG
TTTTCTAAAAGCAATAGGGGTAGTTTAATAATTCTTATATAATCATCATATACACTATTTTTAGTTCTTAATTCTTTAATACA
AACTTATTAATGTGCTCTCCATTGATCTCTTAATCAGGAGGCGATATATACCGGAAGCGGTGTACTTTTCTTCACCTCTTACT
CAACTATGTTGATGTGCAAGTTTAACCACTCGTCGATATTATCTATTGCTATAACGAAAACTTTATTCGAGTTCACAGTGAAA
AACTTCAGCACATTTATGGAAGATCTAAGCAAAATGGAGAACGCCAGTAGATGCGAACAACAAACTTTATCAAATTTGAAATA
CCACTGCTTTGATAAGCTATAGCTTGGCGTAATCATGGTCATAGCTGTTTCCTGTGTGAAATTGTTATCCGCTCACAATTCCA
CACAACATACGAGCCGGAAGCATAAAGTGTAAAGCCTGGGGTGCCTAATGAGTGAGCTAACTCACATTAATTGCGTTGCGCTC
ACTGCCCGCTTTCCAGTCGGGAAACCTGTCGTGCCAGCTGCATTAATGAATCGGCCAACGCGCGGGGAGAGGCGGTTTGCGTA
TTGGGCGCTCTTCCGCTTCCTCGCTCACTGACTCGCTGCGCTCGGTCGTTCGGCTGCGGCGAGCGGTATCAGCTCACTCAAAG
GCGGTAATACGGTTATCCACAGAATCAGGGGATAACGCAGGAAAGAACATGTGAGCAAAAGGCCAGCAAAAGGCCAGGAACCG
TAAAAAGGCCGCGTTGCTGGCGTTTTTCCATAGGCTCCGCCCCCCTGACGAGCATCACAAAAATCGACGCTCAAGTCAGAGGT
GGCGAAACCCGACAGGACTATAAAGATACCAGGCGTTTCCCCCTGGAAGCTCCCTCGTGCGCTCTCCTGTTCCGACCCTGCCG
CTTACCGGATACCTGTCCGCCTTTCTCCCTTCGGGAAGCGTGGCGCTTTCTCATAGCTCACGCTGTAGGTATCTCAGTTCGGT
GTAGGTCGTTCGCTCCAAGCTGGGCTGTGTGCACGAACCCCCCGTTCAGCCCGACCGCTGCGCCTTATCCGGTAACTATCGTC
TTGAGTCCAACCCGGTAAGACACGACTTATCGCCACTGGCAGCAGCCACTGGTAACAGGATTAGCAGAGCGAGGTATGTAGGC
GGTGCTACAGAGTTCTTGAAGTGGTGGCCTAACTACGGCTACACTAGAAGAACAGTATTTGGTATCTGCGCTCTGCTGAAGCC
AGTTACCTTCGGAAAAAGAGTTGGTAGCTCTTGATCCGGCAAACAAACCACCGCTGGTAGCGGTGGTTTTTTTGTTTGCAAGC
AGCAGATTACGCGCAGAAAAAAAGGATCTCAAGAAGATCCTTTGATCTTTTCTACGGGGTCTGACGCTCAGTGGAACGAAAAC
TCACGTTAAGGGATTTTGGTCATGAGATTATCAAAAAGGATCTTCACCTAGATCCTTTTAAATTAAAAATGAAGTTTTAAATC
AATCTAAAGTATATATGAGTAAACTTGGTCTGACAGTTACCAATGCTTAATCAGTGAGGCACCTATCTCAGCGATCTGTCTAT
TTCGTTCATCCATAGTTGCCTGACTCCCCGTCGTGTAGATAACTACGATACGGGAGGGCTTACCATCTGGCCCCAGTGCTGCA
ATGATACCGCGAGACCCACGCTCACCGGCTCCAGATTTATCAGCAATAAACCAGCCAGCCGGAAGGGCCGAGCGCAGAAGTGG
TCCTGCAACTTTATCCGCCTCCATCCAGTCTATTAATTGTTGCCGGGAAGCTAGAGTAAGTAGTTCGCCAGTTAATAGTTTGC
GCAACGTTGTTGCCATTGCTACAGGCATCGTGGTGTCACGCTCGTCGTTTGGTATGGCTTCATTCAGCTCCGGTTCCCAACGA
TCAAGGCGAGTTACATGATCCCCCATGTTGTGCAAAAAAGCGGTTAGCTCCTTCGGTCCTCCGATCGTTGTCAGAAGTAAGTT
GGCCGCAGTGTTATCACTCATGGTTATGGCAGCACTGCATAATTCTCTTACTGTCATGCCATCCGTAAGATGCTTTTCTGTGA
CTGGTGAGTACTCAACCAAGTCATTCTGAGAATAGTGTATGCGGCGACCGAGTTGCTCTTGCCCGGCGTCAATACGGGATAAT
ACCGCGCCACATAGCAGAACTTTAAAAGTGCTCATCATTGGAAAACGTTCTTCGGGGCGAAAACTCTCAAGGATCTTACCGCT
GTTGAGATCCAGTTCGATGTAACCCACTCGTGCACCCAACTGATCTTCAGCATCTTTTACTTTCACCAGCGTTTCTGGGTGAG
CAAAAACAGGAAGGCAAAATGCCGCAAAAAAGGGAATAAGGGCGACACGGAAATGTTGAATACTCATACTCTTCCTTTTTCAA
TATTATTGAAGCATTTATCAGGGTTATTGTCTCATGAGCGGATACATATTTGAATGTATTTAGAAAAATAAACAAATAGGGGT
TCCGCGCACATTTCCCCGAAAAGTGCCACCTGACGTCTAAGAAACCATTATTATCATGACATTAACCTATAAAAATAGGCGTA
TCACGAGGCCCTTTCGTC.

Plasmid 2 can include, for example, a restriction site for integration of a randomized peptide library (such as a randomized NNK 60-mer sequences) driven by a strong promoter (such as the ADH1 promoter) or an inducible promoter (such as the GAL1 promoter). The library can also initiate with a fixed sequence of, for example, Methionine Valine Asparagine (MVN; SEQ ID NO.: 66) or another combination of high-half-life N-end residues (see, for e.g., Varshavsky. Proc. Natl. Acad. Sci. USA. 93:12142-12149 (1996)) to maximize the half-life of the peptide, and terminate with the UTR of a short protein (such as sORF1). The peptide can also be tagged with a protein tag such as Myc. The peptide can also be the product of a ribosomally synthesized and post-translationally modified peptide (RiPP) whereby the core peptide is flanked by prepropeptide sequence comprising a leader peptide and recognition sequences which signal for the recruitment of maturation, cleavage, and/or modifying enzymes such as excision or cyclization enzymes including, for example, lanthipeptides maturation enzymes from Lactococcus lactis (LanB, LanC, LanM, LanP) patellamide biosynthesis factors from cyanobacteria (PatD, PatG), butelase 1 from Clitoria ternatea, and POPB from Galerina marginata or Amanita bisporigera.

Plasmid 2 can additionally encode for one or more of the agents outlined as ‘death agents’ (e.g. cytotoxic reporters) driven by a promoter which depends on the DBD present in the PPI integration plasmid, for example, the TetO sequence which can become bound by TetR. To ensure repression of the ‘death agents,’ Plasmid 2 can include a silencing construct such as a TetR′-Tup11 fusion driven by a strong promoter (such as ADH1) to bind the DBD and silence transcription in the presence of doxycycline. Plasmid 2 can include bacterial selection and propagation markers (i.e. ori and AmpR), and yeast replication and selection markers (i.e. LEU2 and CEN or 2 um). The sequence of Plasmid 2 can be:

(SEQ ID NO.: 64)
TGCATGCCTGCAGGTCGAGATCCGGGATCGAAGAAATGATGGTAAATGAAATAGGAAATCAAGGAGCATGAAGGCAAAAGACAA
ATATAAGGGTCGAACGAAAAATAAAGTGAAAAGTGTTGATATGATGTATTTGGCTTTGCGGCGCCGAAAAAACGAGTTTACGCA
ATTGCACAATCATGCTGACTCTGTGGCGGACCCGCGCTCTTGCCGGCCCGGCGATAACGCTGGGCGTGAGGCTGTGCCCGGCGG
AGTTTTTTGCGCCTGCATTTTCCAAGGTTTACCCTGCGCTAAGGGGCGAGATTGGAGAAGCAATAAGAATGCCGGTTGGGGTTG
CGATGATGACGACCACGACAACTGGTGTCATTATTTAAGTTGCCGAAAGAACCTGAGTGCATTTGCAACATGAGTATACTAGAA
GAATGAGCCAAGACTTGCGAGACGCGAGTTTGCCGGTGGTGCGAACAATAGAGCGACCATGACCTTGAAGGTGAGACGCGCATA
ACCGCTAGAGTACTTTGAAGAGGAAACAGCAATAGGGTTGCTACCAGTATAAATAGACAGGTACATACAACACTGGAAATGGTT
GTCTGTTTGAGTACGCTTTCAATTCATTTGGGTGTGCACTTTATTATGTTACAATATGGAAGGGAACTTTACACTTCTCCTATG
CACATATATTAATCATAAGTTGAATTCGACAGGTTATCAGCAACAACACAGTCATATCCATTCTCAATTAGCTCTACCACAGTG
TGTGAACCAATGTATCCAGCACCACCTGTAACCAAAACAATTTTACCTCGATCGAGTTTACCACTCCCTATCAGTGATAGAGAA
AAGTTAAAGTCGAGTTTACCACTCCCTATCAGTGATAGAGAACAGTCAAAGTCGAGTTTACCACTCCCTATCAGTGATAGAGAA
AAGTTAAAGTCGAGTTTACCACTCCCTATCAGTGATAGAGAAAAGTTAAAGTCGAGTTTACCACTCCCTATCAGTGATAGAGAA
CAGTCAAAGTCGAGTTTACCACTCCCTATCAGTGATAGAGAATTGTGAAAGTCGAGTTTACCACTCCCTATCAGTGATAGAGAA
AAGTCCAAGTCGAGCTCGGTACCCTATGGCATGCATGTGGATGATAATGCGATTAGTTTTTTAGCCTTATTTCTGGGGTAATTA
ATCAGCGAAGCGATGATTTTTGATCTATTAACAGATATATAAATGGAAAAGCTGCATAACCACTTTAACTAATACTTTCAACAT
TTTCAGTTTGTATTACTTCTTATTCAAATGTCATAAAAGTATCAACAAAAAATTGTTAATATACCTCTATACTTTAACGTCAAG
GAGAAAAAACTATAATGCAAAAATCTCATTCAGAAGAAGTGATTGTACCTGAGTTCAATTCTAGCGCAAAGGAATTACCAAGAC
CATGGCCGAAAAGTGCCCGAGCGGTGCTNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNN(TOXIN)TAATAGCATCATGTAATTAGTTATGTCACGCTTACATT
CACGCCCTCCCCCCACATCCGCTCTAACCGAAAAGGAAGGAGTTAGACAACCTGAAGTCTAGGTCCCTATTTATTTTTTTATAG
TTATGTTAGTATTAAGAACGTTATTTATATTTCAAATTTTTCTTTTTTTTCTGTACAGACGCGTGTACGCATGTAACATTATAC
TGAAAACCTTGCTTGAGAAGGTTTTGGGACGCTCGAAGGCTTTAATTTGATACGGATTAGAAGCCGCCGAGCGGGCGACAGCCC
TCCGACGGAAGACTCTCCTCCGTGCGTCCTCGTCTTCACCGGTCGCGTTCCTGAAACGCAGATGTGCCTCGCGCCGCACTGCTC
CGAACAATAAAGATTCTACAATACTAGCTTTTATGGTTATGAAGAGGAAAAATTGGCAGTAACCTGGCCCCACAAACCTTCAAA
TTAACGAATCAAATTAACAACCATAGGATGATAATGCGATTAGTTTTTTAGCCTTATTTCTGGGGTAATTAATCAGCGAAGCGA
TGATTTTTGATCTATTAACAGATATATAAATGGAAAAGCTGCATAACCACTTTAACTAATACTTTCAACATTTTCAGTTTGTAT
TACTTCTTATTCAAATGTCATAAAAGTATCAACAAAAAATTGTTAATATACCTCTATACTTTAACGTCAAGGAGAAAAAACTAT
AATGGTTAATNNNNNNNNNNNNNNNNNNNNNNNNNNNNNNAATAGACAAGCTACGTTGAAACAAGAACCCGCCTCCTTTCAGAA
CTCACTTACGGTACATTAGCGATGACTACGACTTCATTACTCTTTTTTTCAGAAAATTTTAATCAATATTCATTTATTCTACGA
ACAGTCTCCTTCACCTTAGTTTCTTTCTCTGCTCCTTTGAAACTATTATTGTATTTGGTACATTTTAGAGAAAATAAAACATAT
ATAGAACAATGAGAAGGTACGTAATTTCTTAGCTAATTATTTGTAATCAATTAAGCGTCTTCCTTTAGCAAAGCGTCTCTCTTT
TCAGCAACTCTTTGAGCTCTTCTGTCACGAGCAGCTCTGTTCTTCAATCTTCTAGCTTCAGCTTCTTCGTTCAAAGCCTTTTCA
CGTTGAGCATCAGCCTTAGCTTGAATAATGTGTTCAACCAAAGCTCTCTTGTGCTTGAAGGCGTTACCCTTGGATTCCTTGTAC
AAAACGTGGTACAAATGCTTGTCAATCTTACCAGCGTCACGGTACTTGGCCAATAATCTTCTCAAGACACGTAATCTTCTGATC
CAGACGACTTGGGATGGTAAACGAGCTTCTCTAGTACCCTTTCTCTTACCGTAACCACTGTGACGACCTTCTCTCTTGGATTGA
GCATGGGCTCTAGTTCTAGATTTAGAGTGGACAGTAACGGCCTTCTTGACGATGGTTCCGTTCTTGACCAATTTTCTAATGGCA
TTTCTAGAGTTGGCTTGGGCAATTTCAGAAGTTTCGTTTGGGTCTAACCAAACCTTTCTCTTACCAACACCGACAACAGAAGCG
GCAAGTCTCTTTTGAGTACGCAAGTTAGCCCTGTGAAAAAAAGTTTTTGCAGATTTATTTGCATATTGATGTTAGTAAAGTTGC
TTCATTTTTAAAATCCTGAAACCTAACAGTAAAGAGCATATTCGCAAAGGTTAATGAATTACTTTATCTATCAATCGAATTAAC
GCTTGCAGGAACAGACACGTACCATTGCTGCGATAATTCTATAGTTTGTAATAAACGCGGCAATTCGTACAAGCTTGAAATTTA
TCTGAGGTTCTTCTATGGATGTTGCTACCAACTATGCGACCACCGGATGCTGTATCCTCAATTTTTTTCCTTATCTATTTCTCT
CCAAAGGATGACATTCATAACATATTTAAAGATAAATCTTGTGAAAGGTTCAAAATTTAGTATCACTGTTAAACATACATTTTC
CTCTAATTTATTGGTGACTTTTTATTCGATTTGGTGAAAAGATCTATCAAGTAGCACTAGCGTATAAATGTACTATTAGTATCC
CGATGTAGATACAGTAAGCTTTGGACTTCTTCGCCAGAGGTTTGGTCAAGTCTCCAATCAAGGTTGTCGGCTTGTCTACCTTGC
CAGAAATTTACGAAAAGATGGAAAAGGGTCAAATCGTTGGTAGATACGTTGTTGACACTTCTAAATAAGCGAATTTCTTATGAT
TTATGATTTTTATTATTAAATAAGTTATAAAAAAAATAAGTGTATACAAATTTTAAAGTGACTCTTAGGTTTTAAAACGAAAAT
TCTTATTCTTGAGTAACTCTTTCCTGTAGGTCAGGTTGCTTTCTCAGGTATAGCATGAGGTCGCTCTTATTGACCACACCTCTA
CCGGCCGGTCGAAATTCCCCTACCCTATGAACATATTCCATTTTGTAATTTCGTGTCGTTTCTATTATGAATTTCATTTATAAA
GTTTATGTACAAATATCATAAAAAAAGAGAATCTTTTTAAGCAAGGATTTTCTTAACTTCTTCGGCGACAGCATCACCGACTTC
GGTGGTACTGTTGGAACCACCTAAATCACCAGTTCTGATACCTGCATCCAAAACCTTTTTAACTGCATCTTCAATGGCCTTACC
TTCTTCAGGCAAGTTCAATGACAATTTCAACATCATTGCAGCAGACAAGATAGTGGCGATAGGGTTGACCTTATTCTTTGGCAA
ATCTGGAGCAGAACCGTGGCATGGTTCGTACAAACCAAATGCGGTGTTCTTGTCTGGCAAAGAGGCCAAGGACGCAGATGGCAA
CAAACCCAAGGAACCTGGGATAACGGAGGCTTCATCGGAGATGATATCACCAAACATGTTGCTGGTGATTATAATACCATTTAG
GTGGGTTGGGTTCTTAACTAGGATCATGGCGGCAGAATCAATCAATTGATGTTGAACCTTCAATGTAGGAAATTCGTTCTTGAT
GGTTTCCTCCACAGTTTTTCTCCATAATCTTGAAGAGGCCAAAACATTAGCTTTATCCAAGGACCAAATAGGCAATGGTGGCTC
ATGTTGTAGGGCCATGAAAGCGGCCATTCTTGTGATTCTTTGCACTTCTGGAACGGTGTATTGTTCACTATCCCAAGCGACACC
ATCACCATCGTCTTCCTTTCTCTTACCAAAGTAAATACCTCCCACTAATTCTCTGACAACAACGAAGTCAGTACCTTTAGCAAA
TTGTGGCTTGATTGGAGATAAGTCTAAAAGAGAGTCGGATGCAAAGTTACATGGTCTTAAGTTGGCGTACAATTGAAGTTCTTT
ACGGATTTTTAGTAAACCTTGTTCAGGTCTAACACTACCTGTACCCCATTTAGGACCACCCACAGCACCTAACAAAACGGCATC
AGCCTTCTTGGAGGCTTCCAGCGCCTCATCTGGAAGTGGGACACCTGTAGCTTCGATAGCAGCACCACCAATTAAATGATTTTC
GAAATCGAACTTGACATTGGAACGAACATCAGAAATAGCTTTAAGAACCTTAATGGCTTCGGCTGTGATTTCTTGACCAACGTG
GTCACCTGGCAAAACGACGATCTTCTTAGGGGCAGACATTAGAATGGTATATCCTTGAAATATATATATATATTGCTGAAATGT
AAAAGGTAAGAAAAGTTAGAAAGTAAGACGATTGCTAACCACCTATTGGAAAAAACAATAGGTCCTTAAATAATATTGTCAACT
TCAAGTATTGTGATGCAAGCATTTAGTCATGAACGCTTCTCTATTCTATATGAAAAGCCGGTTCCGGCGCTCTCACCTTTCCTT
TTTCTCCCAATTTTTCAGTTGAAAAAGGTATATGCGTCAGGCGACCTCTGAAATTAACAAAAAATTTCCAGTCATCGAATTTGA
TTCTGTGCGATAGCGCCCCTGTGTGTTCTCGTTATGTTGAGGAAAAAAATAATGGTTGCTAAGAGATTCGAACTCTTGCATCTT
ACGATACCTGAGTATTCCCACAGTTGGGGGATCTCGACTCTAGCTAGAGGATCAATTCGTAATCATGTCATAGCTGTTTCCTGT
GTGAAATTGTTATCCGCTCACAATTCCACACAACATACGAGCCGGAAGCATAAAGTGTAAAGCCTGGGGTGCCTAATGAGTGAG
CTAACTCACATTAATTGCGTTGCGCTCACTGCCCGCTTTCCAGTCGGGAAACCTGTCGTGCCAGCTGATAACTTCGTATAATGT
ATGCTATACGAAGTTATTAGGTCTGAAGAGGAGTTTACGTCCAGCCAAGCTAGCTTGGCTGCAGGTCGAGCGGCCGCGATCCGG
AACCCTTAATATAACTTCGTATAATGTATGCTATACGAAGTTATCAGCTGCATTAATGAATCGGCCAACGCGCGGGGAGAGGCG
GTTTGCGTATTGGGCGCTCTTCCGCTTCCTCGCTCACTGACTCGCTGCGCTCGGTCGTTCGGCTGCGGCGAGCGGTATCAGCTC
ACTCAAAGGCGGTAATACGGTTATCCACAGAATCAGGGGATAACGCAGGAAAGAACATGTGAGCAAAAGGCCAGCAAAAGGCCA
GGAACCGTAAAAAGGCCGCGTTGCTGGCGTTTTTCCATAGGCTCCGCCCCCCTGACGAGCATCACAAAAATCGACGCTCAAGTC
AGAGGTGGCGAAACCCGACAGGACTATAAAGATACCAGGCGTTTCCCCCTGGAAGCTCCCTCGTGCGCTCTCCTGTTCCGACCC
TGCCGCTTACCGGATACCTGTCCGCCTTTCTCCCTTCGGGAAGCGTGGCGCTTTCTCATAGCTCACGCTGTAGGTATCTCAGTT
CGGTGTAGGTCGTTCGCTCCAAGCTGGGCTGTGTGCACGAACCCCCCGTTCAGCCCGACCGCTGCGCCTTATCCGGTAACTATC
GTCTTGAGTCCAACCCGGTAAGACACGACTTATCGCCACTGGCAGCAGCCACTGGTAACAGGATTAGCAGAGCGAGGTATGTAG
GCGGTGCTACAGAGTTCTTGAAGTGGTGGCCTAACTACGGCTACACTAGAAGAACAGTATTTGGTATCTGCGCTCTGCTGAAGC
CAGTTACCTTCGGAAAAAGAGTTGGTAGCTCTTGATCCGGCAAACAAACCACCGCTGGTAGCGGTGGTTTTTTTGTTTGCAAGC
AGCAGATTACGCGCAGAAAAAAAGGATCTCAAGAAGATCCTTTGATCTTTTCTACGGGGTCTGACGCTCAGTGGAACGAAAACT
CACGTTAAGGGATTTTGGTCATGAGATTATCAAAAAGGATCTTCACCTAGATCCTTTTAAATTAAAAATGAAGTTTTAAATCAA
TCTAAAGTATATATGAGTAAACTTGGTCTGACAGTTACCAATGCTTAATCAGTGAGGCACCTATCTCAGCGATCTGTCTATTTC
GTTCATCCATAGTTGCCTGACTCCCCGTCGTGTAGATAACTACGATACGGGAGGGCTTACCATCTGGCCCCAGTGCTGCAATGA
TACCGCGAGACCCACGCTCACCGGCTCCAGATTTATCAGCAATAAACCAGCCAGCCGGAAGGGCCGAGCGCAGAAGTGGTCCTG
CAACTTTATCCGCCTCCATCCAGTCTATTAATTGTTGCCGGGAAGCTAGAGTAAGTAGTTCGCCAGTTAATAGTTTGCGCAACG
TTGTTGCCATTGCTACAGGCATCGTGGTGTCACGCTCGTCGTTTGGTATGGCTTCATTCAGCTCCGGTTCCCAACGATCAAGGC
GAGTTACATGATCCCCCATGTTGTGCAAAAAAGCGGTTAGCTCCTTCGGTCCTCCGATCGTTGTCAGAAGTAAGTTGGCCGCAG
TGTTATCACTCATGGTTATGGCAGCACTGCATAATTCTCTTACTGTCATGCCATCCGTAAGATGCTTTTCTGTGACTGGTGAGT
ACTCAACCAAGTCATTCTGAGAATAGTGTATGCGGCGACCGAGTTGCTCTTGCCCGGCGTCAATACGGGATAATACCGCGCCAC
ATAGCAGAACTTTAAAAGTGCTCATCATTGGAAAACGTTCTTCGGGGCGAAAACTCTCAAGGATCTTACCGCTGTTGAGATCCA
GTTCGATGTAACCCACTCGTGCACCCAACTGATCTTCAGCATCTTTTACTTTCACCAGCGTTTCTGGGTGAGCAAAAACAGGAA
GGCAAAATGCCGCAAAAAAGGGAATAAGGGCGACACGGAAATGTTGAATACTCATACTCTTCCTTTTTCAATATTATTGAAGCA
TTTATCAGGGTTATTGTCTCATGAGCGGATACATATTTGAATGTATTTAGAAAAATAAACAAATAGGGGTTCCGCGCACATTTC
CCCGAAAAGTGCCACCTGACGTCTAAGAAACCATTATTATCATGACATTAACCTATAAAAATAGGCGTATCACGAGGCCCTTTC
GTCTCGCGCGTTTCGGTGATGACGGTGAAAACCTCTGACACATGCAGCTCCCGGAGACGGTCACAGCTTGTCTGTAAGCGGATG
CCGGGAGCAGACAAGCCCGTCAGGGCGCGTCAGCGGGTGTTGGCGGGTGTCGGGGCTGGCTTAACTATGCGGCATCAGAGCAGA
TTGTACTGAGAGTGCACCATAACGCATTTAAGCATAAACACGCACTATGCCGTTCTTCTCATGTATATATATATACAGGCAACA
CGCAGATATAGGTGCGACGTGAACAGTGAGCTGTATGTGCGCAGCTCGCGTTGCATTTTCGGAAGCGCTCGTTTTCGGAAACGC
TTTGAAGTTCCTATTCCGAAGTTCCTATTCTCTAGCTAGAAAGTATAGGAACTTCAGAGCGCTTTTGAAAACCAAAAGCGCTCT
GAAGACGCACTTTCAAAAAACCAAAAACGCACCGGACTGTAACGAGCTACTAAAATATTGCGAATACCGCTTCCACAAACATTG
CTCAAAAGTATCTCTTTGCTATATATCTCTGTGCTATATCCCTATATAACCTACCCATCCACCTTTCGCTCCTTGAACTTGCAT
CTAAACTCGACCTCTACATTTTTTATGTTTATCTCTAGTATTACTCTTTAGACAAAAAAATTGTAGTAAGAACTATTCATAGAG
TGAATCGAAAACAATACGAAAATGTAAACATTTCCTATACGTAGTATATAGAGACAAAATAGAAGAAACCGTTCATAATTTTCT
GACCAATGAAGAATCATCAACGCTATCACTTTCTGTTCACAAAGTATGCGCAATCCACATCGGTATAGAATATAATCGGGGATG
CCTTTATCTTGAAAAAATGCACCCGCAGCTTCGCTAGTAATCAGTAAACGCGGGAAGTGGAGTCAGGCTTTTTTTATGGAAGAG
AAAATAGACACCAAAGTAGCCTTCTTCTAACCTTAACGGACCTACAGTGCAAAAAGTTATCAAGAGACTGCATTATAGAGCGCA
CAAAGGAGAAAAAAAGTAATCTAAGATGCTTTGTTAGAAAAATAGCGCTCTCGGGATGCATTTTTGTAGAACAAAAAAGAAGTA
TAGATTCTTTGTTGGTAAAATAGCGCTCTCGCGTTGCATTTCTGTTCTGTAAAAATGCAGCTCAGATTCTTTGTTTGAAAAATT
AGCGCTCTCGCGTTGCATTTTTGTTTTACAAAAATGAAGCACAGATTCTTCGTTGGTAAAATAGCGCTTTCGCGTTGCATTTCT
GTTCTGTAAAAATGCAGCTCAGATTCTTTGTTTGAAAAATTAGCGCTCTCGCGTTGCATTTTTGTTCTACAAAATGAAGCACAG
ATGCTTCGTTGCT.

Plasmid 3 can be used to confirm expression of the reporters and the successful construction of the strains. Plasmid 3 can include a direct fusion between the AD and DBD. Plasmid 3 can further include bacterial selection and propagation markers (i.e. ori and AmpR), and yeast replication and selection markers (i.e. TRP1 and CEN or 2 um). The sequence of Plasmid 3 can be:

(SEQ ID NO.: 65)
CGGTGCGGGCCTCTTCGCTATTACGCCAGATCCTTTTGTTGTTTCCGGGTGTACAATATGGACTTCCTCTTTTCTGGCAACCAAACC
CATACATCGGGATTCCTATAATACCTTCGTTGGTCTCCCTAACATGTAGGTGGCGGAGGGGAGATATACAATAGAACAGATACCAGA
CAAGACATAATGGGCTAAACAAGACTACACCAATTACACTGCCTCATTGATGGTGGTACATAACGAACTAATACTGTAGCCCTAGAC
TTGATAGCCATCATCATATCGAAGTTTCACTACCCTTTTTCCATTTGCCATCTATTGAAGTAATAATAGGCGCATGCAACTTCTTTT
CTTTTTTTTTCTTTTCTCTCTCCCCCGTTGTTGTCTCACCATATCCGCAATGACAAAAAAATGATGGAAGACACTAAAGGAAAAAAT
TAACGACAAAGACAGCACCAACAGATGTCGTTGTTCCAGAGCTGATGAGGGGTATCTCGAAGCACACGAAACTTTTTCCTTCCTTCA
TTCACGCACACTACTCTCTAATGAGCAACGGTATACGGCCTTCCTTCCAGTTACTTGAATTTGAAATAAAAAAAAGTTTGCTGTCTT
GCTATCAAGTATAAATAGACCTGCAATTATTAATCTTTTGTTTCCTCGTCATTGTTCTCGTTCCCTTTCTTCCTTGTTTCTTTTTCT
GCACAATATTTCAAGCTATACCAAGCATACAATCAACTCCAAGCTTTGCAAAGATGGGGTCAAAGGCCGAGCTAATCCCAGAGCCCC
CTAAAAAAAAGAGAAAGGTCGAGCTGGGAACTGCGGCAGAGTACCCGTATGATGTACCGGACTATGCCGGAGGTATGTCTAGATTGG
ACAAGTCTAAGGTTATCAACTCTGCTTTGGAATTGTTGAACGAAGTTGGTATCGAAGGTTTGACTACTAGAAAGTTGGCTCAAAAGT
TGGGTGTTGAACAACCAACTTTGTACTGGCACGTTAAGAACAAGAGAGCTTTGTTGGACGCTTTGGCTATCGAAATGTTGGACAGAC
ACCACACTCACTTCTGTCCATTGGAAGGTGAATCTTGGCAAGACTTCTTGAGAAACAACGCTAAGTCTTTCAGATGTGCTTTGTTGT
CTCACAGAGACGGTGCTAAGGTTCACTTGGGTACTAGACCAACTGAAAAGCAATACGAAACTTTGGAAAACCAATTGGCTTTCTTGT
GTCAACAAGGTTTCTCTTTGGAAAACGCTTTGTACGCTTTGTCTGCTGTTGGTCACTTCACTTTGGGTTGTGTTTTGGAAGACCAAG
AACACCAAGTTGCTAAGGAAGAAAGAGAAACTCCAACTACTGACTCTATGCCACCATTGTTGAGACAAGCTATCGAATTGTTCGACC
ACCAAGGTGCTGAACCAGCTTTCTTGTTCGGTTTGGAATTGATCATCTGTGGTTTGGAAAAGCAATTGAAGTGTGAATCTGGTTCTG
GGCAACCATCTTTGAGATCTGAATACGAATACCCAGTTTTCTCTCACGTTCAAGCTGGTATGTTCTCTCCAGAATTGAGAACTTTCA
CTAAGGGTGACGCTGAAAGATGGGTTTCTGGTCCAGGTACTGAAGACGCTGAAGCTGTTGCTTTGGGTTTGGGTTTGTCTGACTTCC
CATCTGCTGGTAAGGCTGTTTTGGACGACGAAGACTCTTTCGTTTGGCCAGCTGCTTCTTTCGACATGGGTGCTTGTTGGGCTGGTG
CTGGTTTCGCTGACCCAGACCCAGCTTGTATCTTCTTGAACTTGCCATGAGCCCATCTTTTTTTTGGACCTAAATTCTTCATGAAAA
TATATTACGAGGGCTTATTCAGAAGCTTTGGACTTCTTCGCTTGCAGCCAAGCTAATTCCGGGCGAATTTCTTATGATTTATGATTT
TTATTATTAAATAAGTTATAAAAAAAATAAGTGTATACAAATTTTAAAGTGACTCTTAGGTTTTAAAACGAAAATTCTTATTCTTGA
GTAACTCTTTCCTGTAGGTCAGGTTGCTTTCTCAGGTATAGCATGAGGTCGCTCTTATTGACCACACCTCTACCGGCATGCAAGCTT
GGCGTAATCATGGTCATAGCTGTTTCCTGTGTGAAATTGTTATCCGCTCACAATTCCACACAACATACGAGCCGGAAGCATAAAGTG
TAAAGCCTGGGGTGCCTAATGAGTGAGCTAACTCACATTAATTGCGTTGCGCTCACTGCCCGCTTTCCAGTCGGGAAACCTGTCGTG
CCAGCTGCATTAATGAATCGGCCAACGCGCGGGGAGAGGCGGTTTGCGTATTGGGCGCTCTTCCGCTTCCTCGCTCACTGACTCGCT
GCGCTCGGTCGTTCGGCTGCGGCGAGCGGTATCAGCTCACTCAAAGGCGGTAATACGGTTATCCACAGAATCAGGGGATAACGCAGG
AAAGAACATGTGAGCAAAAGGCCAGCAAAAGGCCAGGAACCGTAAAAAGGCCGCGTTGCTGGCGTTTTTCCATAGGCTCCGCCCCCC
TGACGAGCATCACAAAAATCGACGCTCAAGTCAGAGGTGGCGAAACCCGACAGGACTATAAAGATACCAGGCGTTTCCCCCTGGAAG
CTCCCTCGTGCGCTCTCCTGTTCCGACCCTGCCGCTTACCGGATACCTGTCCGCCTTTCTCCCTTCGGGAAGCGTGGCGCTTTCTCA
TAGCTCACGCTGTAGGTATCTCAGTTCGGTGTAGGTCGTTCGCTCCAAGCTGGGCTGTGTGCACGAACCCCCCGTTCAGCCCGACCG
CTGCGCCTTATCCGGTAACTATCGTCTTGAGTCCAACCCGGTAAGACACGACTTATCGCCACTGGCAGCAGCCACTGGTAACAGGAT
TAGCAGAGCGAGGTATGTAGGCGGTGCTACAGAGTTCTTGAAGTGGTGGCCTAACTACGGCTACACTAGAAGAACAGTATTTGGTAT
CTGCGCTCTGCTGAAGCCAGTTACCTTCGGAAAAAGAGTTGGTAGCTCTTGATCCGGCAAACAAACCACCGCTGGTAGCGGTGGTTT
TTTTGTTTGCAAGCAGCAGATTACGCGCAGAAAAAAAGGATCTCAAGAAGATCCTTTGATCTTTTCTACGGGGTCTGACGCTCAGTG
GAACGAAAACTCACGTTAAGGGATTTTGGTCATGAGATTATCAAAAAGGATCTTCACCTAGATCCTTTTAAATTAAAAATGAAGTTT
TAAATCAATCTAAAGTATATATGAGTAACCTGAGGCTATGGCAGGGCCTGCCGCCCCGACGTTGGCTGCGAGCCCTGGGCCTTCACC
CGAACTTGGGGGGTGGGGTGGGGAAAAGGAAGAAACGCGGGCGTATTGGCCCCAATGGGGTCTCGGTGGGGTATCGACAGAGTGCCA
GCCCTGGGACCGAACCCCGCGTTTATGAACAAACGACCCAACACCGTGCGTTTTATTCTGTCTTTTTATTGCCGTCATAGCGCGGGT
TCCTTCCGGTATTGTCTCCTTCCGTGTTTCAGTTAGCCTCCCCCTAGGGTGGGCGAAGAACTCCAGCATGAGATCCCCGCGCTGGAG
GATCATCCAGCCGGCGTCCCGGAAAACGATTCCGAAGCCCAACCTTTCATAGAAGGCGGCGGTGGAATCGAAATCTCGTGATGGCAG
GTTGGGCGTCGCTTGGTCGGTCATTTCGAACCCCAGAGTCCCGCTCAGAAGAACTCGTCAAGAAGGCGATAGAAGGCGATGCGCTGC
GAATCGGGAGCGGCGATACCGTAAAGCACGAGGAAGCGGTCAGCCCATTCGCCGCCAAGCTCTTCAGCAATATCACGGGTAGCCAAC
GCTATGTCCTGATAGCGGTCCGCCACACCCAGCCGGCCACAGTCGATGAATCCAGAAAAGCGGCCATTTTCCACCATGATATTCGGC
AAGCAGGCATCGCCATGAGTCACGACGAGATCCTCGCCGTCGGGCATGCTCGCCTTGAGCCTGGCGAACAGTTCGGCTGGCGCGAGC
CCCTGATGCTCTTCGTCCAGATCATCCTGATCGACAAGACCGGCTTCCATCCGAGTACGTGCTCGCTCGATGCGATGTTTCGCTTGG
TGGTCGAATGGGCAGGTAGCCGGATCAAGCGTATGCAGCCGCCGCATTGCATCAGCCATGATGGATACTTTCTCGGCAGGAGCAAGG
TGAGATGACAGGAGATCCTGCCCCGGCACTTCGCCCAATAGCAGCCAGTCCCTTCCCGCTTCAGTGACAACGTCGAGCACAGCTGCG
CAAGGAACGCCCGTCGTGGCCAGCCACGATAGCCGCGCTGCCTCGTCTTGCAGTTCATTCAGGGCACCGGACAGGTCGGTCTTGACA
AAAAGAACCGGGCGCCCCTGCGCTGACAGCCGGAACACGGCGGCATCAGAGCAGCCGATTGTCTGTTGTGCCCAGTCATAGCCGAAT
AGCCTCTCCACCCAAGCGGCCGGAGAACCTGCGTGCAATCCATCTTGTTCAATCATACTCTTCCTTTTTCAATATTATTGAAGCATT
TATCAGGGTTATTGTCTCATGAGCGGATACATATTTGAATGTATTTAGAAAAATAAACAAATAGGGGTTCCGCGCACATTTCCCCGA
AAAGTGCCACCTGAACGAAGCATCTGTGCTTCATTTTGTAGAACAAAAATGCAACGCGAGAGCGCTAATTTTTCAAACAAAGAATCT
GAGCTGCATTTTTACAGAACAGAAATGCAACGCGAAAGCGCTATTTTACCAACGAAGAATCTGTGCTTCATTTTTGTAAAACAAAAA
TGCAACGCGAGAGCGCTAATTTTTCAAACAAAGAATCTGAGCTGCATTTTTACAGAACAGAAATGCAACGCGAGAGCGCTATTTTAC
CAACAAAGAATCTATACTTCTTTTTTGTTCTACAAAAATGCATCCCGAGAGCGCTATTTTTCTAACAAAGCATCTTAGATTACTTTT
TTTCTCCTTTGTGCGCTCTATAATGCAGTCTCTTGATAACTTTTTGCACTGTAGGTCCGTTAAGGTTAGAAGAAGGCTACTTTGGTG
TCTATTTTCTCTTCCATAAAAAAAGCCTGACTCCACTTCCCGCGTTTACTGATTACTAGCGAAGCTGCGGGTGCATTTTTTCAAGAT
AAAGGCATCCCCGATTATATTCTATACCGATGTGGATTGCGCATACTTTGTGAACAGAAAGTGATAGCGTTGATGATTCTTCATTGG
TCAGAAAATTATGAACGGTTTCTTCTATTTTGTCTCTATATACTACGTATAGGAAATGTTTACATTTTCGTATTGTTTTCGATTCAC
TCTATGAATAGTTCTTACTACAATTTTTTTGTCTAAAGAGTAATACTAGAGATAAACATAAAAAATGTAGAGGTCGAGTTTAGATGC
AAGTTCAAGGAGCGAAAGGTGGATGGGTAGGTTATATAGGGATATAGCACAGAGATATATAGCAAAGAGATACTTTTGAGCAATGTT
TGTGGAAGCGGTATTCGCAATATTTTAGTAGCTCGTTACAGTCCGGTGCGTTTTTGGTTTTTTGAAAGTGCGTCTTCAGAGCGCTTT
TGGTTTTCAAAAGCGCTCTGAAGTTCCTATACTTTCTAGAGAATAGGAACTTCGGAATAGGAACTTCAAAGCGTTTCCGAAAACGAG
CGCTTCCGAAAATGCAACGCGAGCTGCGCACATACAGCTCACTGTTCACGTCGCACCTATATCTGCGTGTTGCCTGTATATATATAT
ACATGAGAAGAACGGCATAGTGCGTGTTTATGCTTAAATGCGTACTTATATGCGTCTATTTATGTAGGATGAAAGGTAGTCTAGTAC
CTCCTGTGATATTATCCCATTCCATGCGGGGTATCGTATGCTTCCTTCAGCACTACCCTTTAGCTGTTCTATATGCTGCCACTCCTC
AATTGGATTAGTCTCATCCTTCAATGCTATCATTTCCTTTGATATTGGATCATACTAAGAAACCATTATTATCATGACATTAACCTA
TAAAAATAGGCGTATCACGAGGCCCTTTCGTCTCGCGCGTTTCGGTGATGACGGTGAAAACCTCTGACACATGCAGCTCCCGGAGAC
GGTCACAGCTTGTCTGTAAGCGGATGCCGGGAGCAGACAAGCCCGTCAGGGCGCGTCAGCGGGTGTTGGCGGGTGTCGGGGCTGGCT
TAACTATGCGGCATCAGAGCAGATTGTACTGAGAGTGCACCATAGATCAACGACATTACTATATATATAATATAGGAAGCATTTAAT
AGAACAGCATCGTAATATATGTGTACTTTGCAGTTATGACGCCAGATGGCAGTAGTGGAAGATATTCTTTATTGAAAAATAGCTTGT
CACCTTACGTACAATCTTGATCCGGAGCTTTTCTTTTTTTGCCGATTAAGAATTAATTCGGTCGAAAAAAGAAAAGGAGAGGGCCAA
GAGGGAGGGCATTGGTGACTATTGAGCACGTGAGTATACGTGATTAAGCACACAAAGGCAGCTTGGAGTATGTCTGTTATTAATTTC
ACAGGTAGTTCTGGTCCATTGGTGAAAGTTTGCGGCTTGCAGAGCACAGAGGCCGCAGAATGTGCTCTAGATTCCGATGCTGACTTG
CTGGGTATTATATGTGTGCCCAATAGAAAGAGAACAATTGACCCGGTTATTGCAAGGAAAATTTCAAGTCTTGTAAAAGCATATAAA
AATAGTTCAGGCACTCCGAAATACTTGGTTGGCGTGTTTCGTAATCAACCTAAGGAGGATGTTTTGGCTCTGGTCAATGATTACGGC
ATTGATATCGTCCAACTGCATGGAGATGAGTCGTGGCAAGAATACCAAGAGTTCCTCGGTTTGCCAGTTATTAAAAGACTCGTATTT
CCAAAAGACTGCAACATACTACTCAGTGCAGCTTCACAGAAACCTCATTCGTTTATTCCCTTGTTTGATTCAGAAGCAGGTGGGACA
GGTGAACTTTTGGATTGGAACTCGATTTCTGACTGGGTTGGAAGGCAAGAGAGCCCCGAAAGCTTACATTTTATGTTAGCTGGTGGA
CTGACGCCAGAAAATGTTGGTGATGCGCTTAGATTAAATGGCGTTATTGGTGTTGATGTAAGCGGAGGTGTGGAGACAAATGGTGTA
AAAGACTCTAACAAAATAGCAAATTTCGTCAAAAATGCTAAGAAATAGGTTATTACTGAGTAGTATTTATTTAAGTATTGTTTGTGC
ACTTGCCGATCTATGCGGTGTGAAATACCGCACAGATGCGTAAGGAGAAAATACCGCATCAGGAAATTGTAAGCGTTAATATTTTGT
TAAAATTCGCGTTAAATTTTTGTTAAATCAGCTCATTTTTTAACCAATAGGCCGAAATCGGCAAAATCCCTTATAAATCAAAAGAAT
AGACCGAGATAGGGTTGAGTGTTGTTCCAGTTTGGAACAAGAGTCCACTATTAAAGAACGTGGACTCCAACGTCAAAGGGCGAAAAA
CCGTCTATCAGGGCGATGGCCCACTACGTGAACCATCACCCTAATCAAGTTTTTTGGGGTCGAGGTGCCGTAAAGCACTAAATCGGA
ACCCTAAAGGGAGCCCCCGATTTAGAGCTTGACGGGGAAAGCCGGCGAACGTGGCGAGAAAGGAAGGGAAGAAAGCGAAAGGAGCGG
GCGCTAGGGCGCTGGCAAGTGTAGCGGTCACGCTGCGCGTAACCACCACACCCGCCGCGCTTAATGCGCCGCTACAGGGCGCGTCCA
TTCGCCATTCAGGCTGCGCAACTGTTGGGAAGGGCGAT.

The host cell used for identification of the peptide that disrupts a PPI can be Saccharomyces cerevisiae of either mating type or a diploid, and include the genomic integration of genetic reporters (such as ADE2, HIS3, and/or URA3) driven by the recognition sequence of the DBD used in Plasmid 1 (such as TetO). The host cell used for identification of the peptide that disrupts a PPI can also be another host cell as previously described herein, such as a bacterial cell (e.g. E. coli, B. subtilis) or a mammalian cell (e.g. immortalized primary cells or immortalized cell lines). The host cell can also express enzymes necessary for the cyclization and methylation of peptides (e.g. lanthipeptides maturation enzymes from Lactococcus lactis (LanB, LanC, LanM, LanP), patellamide biosynthesis factors from cyanobacteria (PatD, PatG), butelase 1 from Clitoria ternatea, or POPB from Galerina marginata or Amanita bisporigera. Exemplary POPs necessary for cyclization of peptides can also comprise any of the sequences outlined below in Table 2.

TABLE 2

| | Sequence | |
|---|---|---|
| Species/Gene | Reference No | Amino Acid Sequence |
| Amanita | GenBank | MPPTPWAPHS YPPTRRSDHV DVYQSASRGE VPVPDPYQWL |
| bisporigera | HQ225841.1 | EENSNEVDEW TTAQTAFTQG YLDKNADRQK LEEKFRASKD |
| POPB | | YVKFSAPTLL DSGHWYWFYN SGVQSQAVLY RSKKPVLPDF |
| | | QRGTRKVGEV YFDPNVLSAD GTAIMGTCRF SPSGEYFAYA |
| | | VSHLGVDYFT IYVRPTSSSL SQAPEAEGGD GRLSDGVKWC |
| | | KFTTITWTKD SKGFLYQRYP ARESLVAKDR DKDAMVCYHR |
| | | VGTTQLEDII VQQDKENPDW TYGTDASEDG KYIYLVVYKD |
| | | ASKQNLLWVA EFDKDGVKPE IPWRKVINEF GADYHVITNH |
| | | GSLIYVKTNV NAPQYKVVTI DLSTGEPEIR DFIPEQKDAK |
| | | LTQVKCVNKG YFVAIYKRNV KDEIYLYSKA GDQLSRLASD |
| | | FIGVASITNR EKQPHSFLTF SGFNTPGTIS RYDFTAPDTQ |
| | | RLSILRTTKL NGLNADDFES TQVWYKSKDG TKVPMFIVRH |
| | | KSTKFDGTAP AIQNGYGGFA ITADPFFSPI MLTFMQTYGA |
| | | ILAVPNIRGG GEFGGEWHKA GRRETKGNTF DDFIAAAQFL |
| | | VKNKYAAPGK VAITGASNGG FLVCGSVVRA PEGTFGAAVS |
| | | EGGVADLLKF NKFTGGMAWT SEYGNPFIKE DFDFVQALSP |
| | | VHNVPKDRVL PATLLMTNAG DDRVVPMHSL KFVANLQYNV |
| | | PQNPHPLLIR VDKSWLGHGF GKTTDKHTKD AADKWSFVAQ |
| | | SLGLEWKTVD (SEQ ID NO: 83) |
| Galerina | GenBank | MSSVTWAPGN YPSTRRSDHV DTYQSASKGE VPVPDPYQWL |
| marginata | JN827314.2 | EESTDEVDKW TTAQADLAQS YLDQNADIQK LAEKFRASRN |
| POPB | | YAKFSAPTLL DDGHWYWFYN RGLQSQSVLY RSNEPALPDF |
| | | SNGDDNVGDV FFDPNVLATD GSAGMVLCKF SPDGKFFAYA |
| | | VSHLGGDYST IYIRSTSSPL SQASAVQGTD GRLSDEVKWF |
| | | KFSTIIWTKD SKGFLYQRYP ARERHEGTRS DRNAMMCYHK |
| | | VGTTQEEDII VYQDNEHPEW IYGADTSEDG KYLYLYQFKD |
| | | TSKKNLLWVA ELNEDGVKSG IQWRKVVNEY VADYNVITNH |
| | | GSLVYIKTNL NAPQYKVITI DLSKDEPEIR DFIPEEKDAK |
| | | LAQVNCANEE YFVAIYKRNV KDEIYLYSKA GVQLTRLAPD |
| | | FVGAASIANR QKQTHFFLTL SGFNTPGTIA RYDFTAPETQ |
| | | RFSILRTTKV NELDPDDFES TQVWYESKDG TKIPMFIVRH |
| | | KSTKFDGTAA AIQYGYGGFA TSADPFFSPI ILTFLQTYGA |
| | | IFAVPSIRGG GEFGEEWHKG GRRETKVNTF DDFIAAAQFL |
| | | VKNKYAAPGK VAINGASNGG LLVMGSIVRA PEGTFGAAVP |
| | | EGGVADLLKF HKFTGGQAWI SEYGNPSIPE EFDYIYPLSP |
| | | VHNVRTDKVM PATLITVNIG DGRVVPMHSF KFIATLQHNV |
| | | PQNPHPLLIK IDKSWLGHGM GKPTDKNVKD AADKWGFIAR |
| | | ALGLELKTVE (SEQ ID NO: 84) |

Envisioned within the scope of this disclosure are kits comprising Plasmid 1, Plasmid 2, Plasmid 3, transfectable host cells compatible with Plasmids 1-3, or any combination thereof. In some embodiments, Plasmids 1 or 2 are provided already transfected into host cells. In some embodiments, kits according to this disclosure include selectable agents for use with host cells transfected with Plasmids 1-3. In some embodiments a library of variants of Plasmid 1 are provided, wherein more than a single pair of Y2H interactors are represented. Such a library can be used to, for example, screen for protein-protein interactions that are inhibited by a defined agent. In some embodiments a library of variants of Plasmid 1 are provided, wherein a plurality of different short test polypeptide sequences for screening are represented. The plurality of different short peptide sequences can be randomly generated by any method (e.g. NNK or NNN nucleotide randomization). The plurality of different short peptide sequences can also be preselected, either by previous experiments selecting for binding to a target, or from existing data sets in the scientific literature that have reported rationally-designed peptide libraries.

Plasmids 1, 2, and 3 can be used in various permutations. In a first example, integration of Plasmid 1 into the genome of the host cell (as confirmed using Plasmid 3) can be followed by transformation of a library of Plasmid 2 with randomly encoded peptides using, for example, NNK or NNN codons.

In this first example, to perform a screen to identify a peptide that can disrupt a PPI, the host cell 1 can be propagated in selection media to ensure the presence of Plasmid 1 and that a faithful PPI is occurring (e.g. on media lacking Trp for yeast, or in media containing antibiotic for human or bacterial cells). This host cell can then be transformed with Plasmid 2, and immediately be transferred to selection media to ensure all components are present (i.e. on media lacking Trp and Leu for yeast, or antibiotics for bacterial or mammalian cells), and are inducing expression of any inducible component (e.g. with Gal, doxycycline, etc).

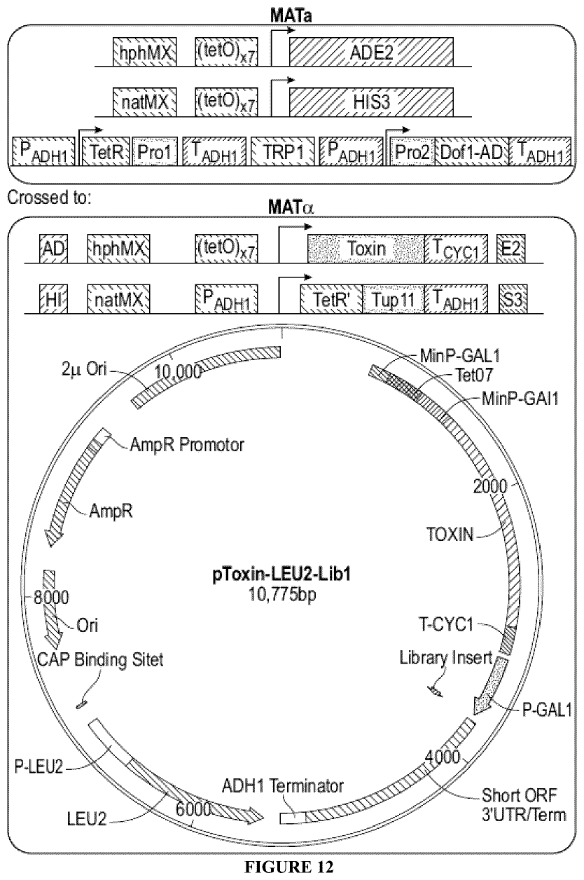

In a second example, the plasmids can be used as a ‘plug and play platform’ utilizing the yeast mating type system (FIG. 12), where the 2 or more plasmids (or the genetic elements therein) can be introduced into the same cell by cell fusion or cell fusion followed by meiosis instead of transfection. This cell fusion involves two different yeast host cells bearing different genetic elements. In this method, yeast host cell 1 can be one of MATa or MATalpha and include an integration of Plasmid 1. This strain can be propagated on positive selection media to ensure a faithful PPI is present. Also in this method, the yeast host cell 2 is of the opposite mating type. This strain carries (or has integrated) the randomized peptide library and ‘death agent’ (e.g. cytotoxic reporter) plasmid (Plasmid 2). Yeast host cell 2 can be generated via large batch high efficiency transformation protocols which ensure a highly diversified library variation within the cell culture. Aliquots of this library batch can then be frozen to maintain consistency.

In this second example, the strains are mated in batch to result in a diploid strain that carries all the markers, the PPI, ‘death agents’ and peptide. This batch culture then can be propagated on solid medium that enables selection of all the system components (i.e. media lacking Leu and Trp), and inducing expression of any inducible component (i.e. with Gal).

Surviving colonies from limiting dilution experiments performed on host cells bearing both the Y2H and library/cytotoxic constructs (either introduced to the cell by transfection or mating) constitute colonies with a PPI that has been disrupted by a peptide and no longer triggers the death cascade triggered by the encoded ‘death agents’ (e.g. cytotoxic reporters). The peptide sequence can be obtained by DNA sequencing the peptide-encoding region of Plasmid 2 in each surviving colony.

To ensure that survival is due to inhibition of the PPI rather than stochastic chance or faulty gene expression, the inducible marker can be used to inactivate the production of either the PPI or the peptide and confirm specificity. For example, observation of cell survival only on media with galactose wherein all the components are expressed, and no survival on media without galactose when expression of the peptide is lost.

The plasmid can also be isolated and re-transformed into a fresh host cell to confirm specificity. Biochemical fractionation of the viable host cells which contains the PPI, peptide and ‘death agent’ followed by pull-down experiments can confirm an interaction between the peptide sequence and either PPI partner using the encoded tags (e.g. Myc-tag, HA-tag, His-tag).

Once enough surviving host cell colonies are sequenced, highly conserved sequence patterns can emerge and can be readily identified using a multiple-sequence alignment. Any such pattern can be used to ‘anchor’ residues within the library peptide insert sequence and permute the variable residues to generate diversity and achieve tighter binding. This can also be done using an algorithm developed for pattern recognition and library design. Upon convergence, the disrupting peptide pattern, as identified through sequencing, can be used to define a necessary and sufficient peptide disruptor sequence. Convergence is defined by the lack of retrieval of any new sequences in the last iteration relative to the penultimate one.

### Compounds

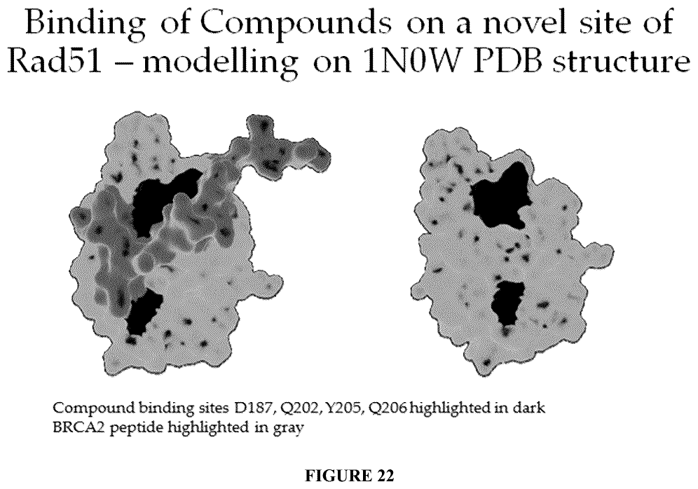

Disclosed herein are non-naturally occurring compounds that can bind to a protein interface of RAD51 and inhibit the function of RAD51 in vitro or in cells. The protein interface can be a subregion of the ATPase domain of RAD51. The protein interface can be RAD51AP1's binding site on RAD51. The protein interface can be amino acid residues 190-218 of human RAD51. An exemplary model of a protein interface where non-naturally occurring compounds according to the invention can bind on RAD51 is presented in FIG. 22, wherein compound interacts with residues highlighted in black. Gray shows the binding of BRCA2 to RAD51.

In vitro, compounds as disclosed herein can inhibit RAD51 multimerization, RAD51 interaction with another known interacting partner of RAD51 (e.g. BRCA2 or RAD51AP1), or RAD51 chelation/binding of Ca2+ ions. The inhibition of interaction with another known interacting partner of RAD51 may be competitive or allosteric. Inhibition of RAD51 multimerization, RAD51 interaction with another known interacting partner of RAD51 (e.g. BRCA2 or RAD51AP1), or RAD51 chelation/binding of Ca2+ ions may be accompanied by inhibition of RAD51 ATPase activity. Inhibition of RAD51 multimerization, RAD51 interaction with another known interacting partner of RAD51 (e.g. BRCA2 or RAD51AP1), or RAD51 chelation/binding of Ca2+ ions may be without inhibition of RAD51 ATPase activity.

In cells, compounds as disclosed herein can inhibit assembly of RAD51 filaments on DNA. In cells, compounds as disclosed herein can inhibit DNA damage repair. In cells, compounds as disclosed herein can inhibit cellular homologous recombination. In cells, compounds as disclosed herein can result in sensitization to genotoxic chemotherapeutics of cancer cells. In cells, compounds as disclosed herein can reduce drug resistance to chemotherapeutic agents, either through inhibition of DNA damage repair, or through inducing cellular stress through increase in intracellular free calcium concentration. In cells that depend on RAD51 overexpression, compounds as disclosed herein can result in cell death. In cells, compounds as disclosed herein may cause death in cellular conditions depending on RAD51 overexpression. In some embodiments, cells can be cancer cells or cells of patients with inheritable benign proliferative disorders (e.g. Cowden's syndrome). Further, any of the compounds disclosed herein can be used in combination with, for example, immuno-oncology agents or PARP inhibitors, or other chemotherapeutics for the purpose of inducing cell death.

The compounds disclosed herein can be used in methods of treatment of a disorder or condition where cell growth inhibition occurs by downregulation of homologous recombination, or where there is overexpression of proteins involved in the DNA damage repair pathway. The compounds disclosed herein can be used in methods of treatment of a disorder or condition associated with aberrant RAD51 activity.

The interface of RAD51 that can be targeted by a compound of the invention can be important for the control of the activity of the BRCA1/2 homologous recombination DNA repair pathway. Blocking of this interface important for the control of the activity of the BRCA1/2 homologous recombination DNA repair pathway with the compounds disclosed herein can have clinical relevance for several therapeutic indications.

A compound disclosed herein can be used as a monotherapy for the treatment of, for example, intrahepatic cholangiocarcinoma (ICC), metastatic castration-resistant prostate cancer (mCRPC), and other cancers that display upregulation of effectors of the RAD51/BRCA2 DNA damage repair pathway.

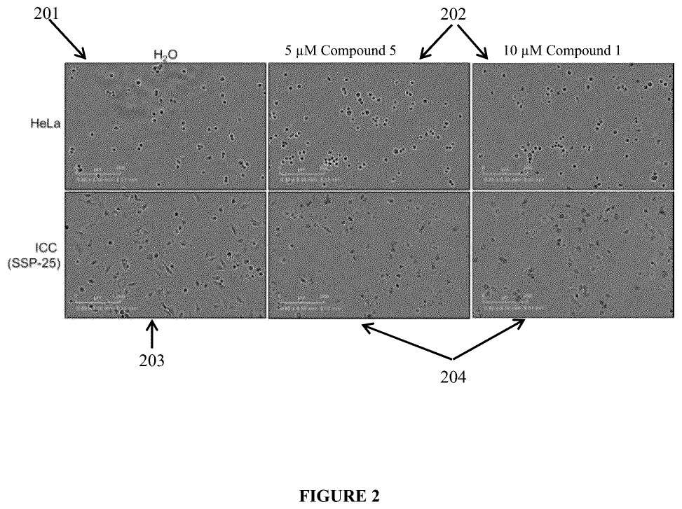

ICC cells can depend on the BRCA2 pathway for survival and render subjects untreatable. The inactivation of the homologous recombination pathway through RAD51 disrupting compounds can lead to cell death. This cell death can be selective for cells that depend on the BRCA2 pathway such as ICC cells. An example of this kind of selectivity is demonstrated in FIG. 2, wherein both HeLa cells (cervical cancer cells) and SSP-25 cells (ICC-derived cells) are treated with compounds 1 and 5 and little cell death is generated in the HeLa cells compared to the SSP-25 cells upon compound treatment

In castration-resistant prostate cancers that overexpress the BRCA2 pathway, the inactivation of BRCA2 via inhibition of RAD51 disrupting compounds disclosed herein can lead to cell death. Several cancers exhibit upregulation of the homologous recombination pathway, specifically of RAD51 and BRCA2, which can render the cancer cells resistant to genotoxic chemotherapy. Cancers that spontaneously overexpress the RAD51/BRCA2 pathway components include, for example, hepatocellular carcinoma, acute myeloid leukemia (AML), aggressive mantle cell lymphoma, ovarian cancer, and imatinib-resistant BCR/ABL cancers.

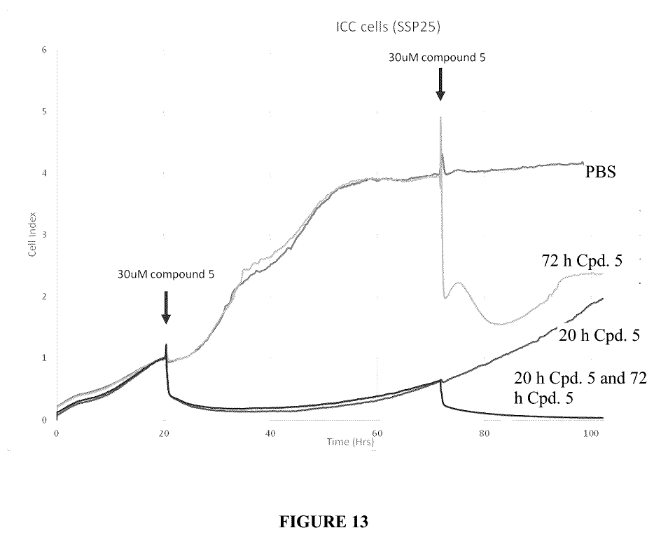

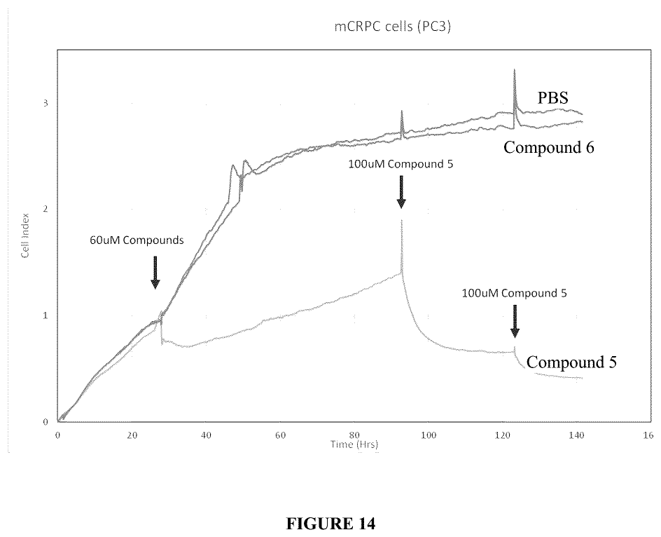

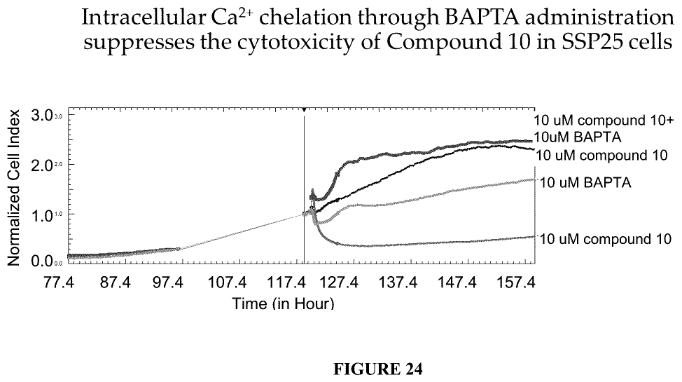

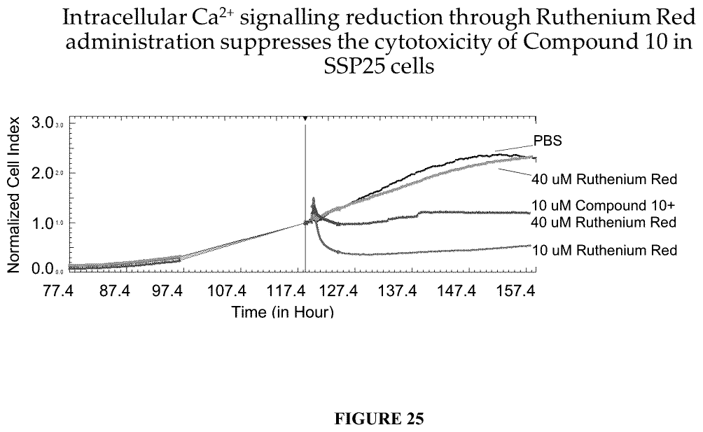

The mechanism of action of the compounds disclosed herein can involve, for example, dislodging a pair of calcium ions coordinated by the RAD51 polymer in an acute manner leading to an acute intracellular free calcium concentration increase. The increase in the intracellular free calcium concentration can lead to cell death. This mechanism of cell death can occur in cells overexpressing the RAD51 protein, and the cell death can be orders of magnitude higher than cells without RAD51 overexpression. The cell death in cells contacted with compounds disclosed herein that overexpress the RAD51 protein may be at least 3, at least 10, at least 50, or at least 100 times greater than in cells contacted with compounds disclosed herein that do not overexpress RAD51. The sequestering of calcium ions by the RAD51 filaments to, for example, micromolar levels and the subsequent acute release of the pool of calcium ions into the cytosol upon compound binding can result in cell death in cancers dependent on the RAD51/BRCA2 pathway such as ICC and mCRPC. Evidence for this sequestration/release mechanism can be seen in FIGS. 24 and 25, wherein the intracellular calcium chelators BAPTA-AM and Ruthenium red are able to counteract the cell-death inducing effects of compound 10, suggesting that the cell death mechanism of compound 10 involves the release of Ca2+ ions. This cell death can occur acutely in a few minutes and can be p53- and cell cycle-independent. An example of the rapidity of this cell death can be seen in FIGS. 13 and 14, where administration of compound (arrows) causes rapid cell death (decrease in cell index).

The mechanism of action of the compounds disclosed herein in cell death can involve inactivation of the recombination pathway through RAD51 alongside increases in cellular free calcium concentration. The mechanism of action of the compounds in cell death can involve increases in cellular free calcium concentration without inhibition of the recombination pathway through RAD51. The mechanism of action of the compounds in cell death can involve inhibition of RAD51 ATPase activity alongside increases in cellular free calcium concentration. The mechanism of action of the compounds in cell death can involve increases in cellular free calcium concentration without inhibition RAD51 ATPase activity.

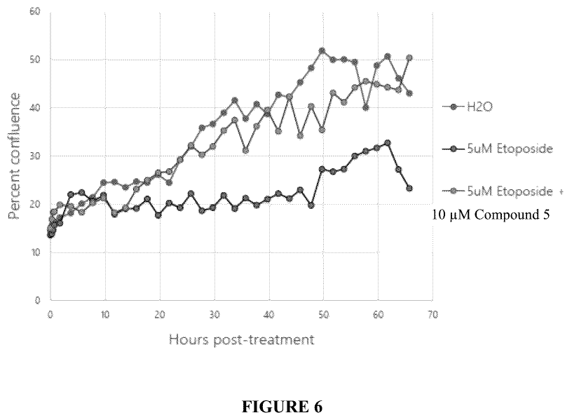

The compounds can also be used in the treatment of rare and orphan diseases including, for example, Bloom's syndrome, Fanconi Anemia, Werner's syndrome, and Nijmegen Breakage Syndrome, which can display an increase in homologous recombination in their patients' cells. Downregulation of recombination by administration of RAD51 disrupting compounds as disclosed herein to individuals' cells can reverse the DNA damage hypersensitivity of the cells. Downregulation of recombination by contact of the RAD51 disrupting compounds as disclosed herein to the cells can reverse the DNA damage hypersensitivity of the cells. An example of this phenomenon can be seen in FIG. 6, where compound 5 is able to rescue the hypersensitivity of a Bloom's cell line (GM08505) to a genotoxic stress (etoposide).

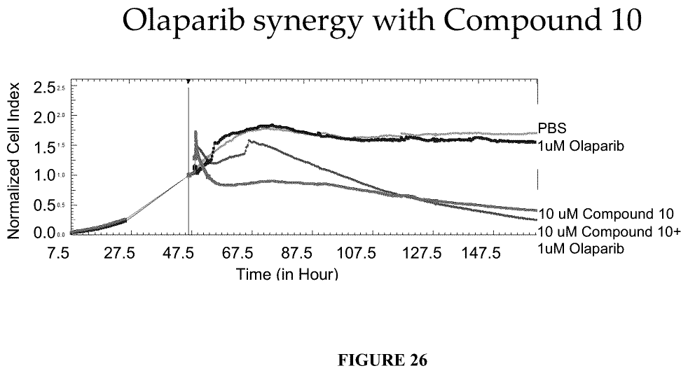

The compounds can also be used in combination with other therapeutic agents including, for example, immuno-oncology agents, PARP inhibitors, and canonical chemotherapeutics. For example, metastatic melanoma patients who are responsive to anti-PD1 therapy can be highly enriched for somatic mutations in the BRCA2 gene within the patient tumors. This correlation can indicate that inactivation of the BRCA2 pathway sensitizes cells to anti-PD1 immunotherapy. The anti-PD1 agents can be, for example, nivolumab, pembrolizumab, or pidilizumab. An example of synergy of compounds as described herein with other therapeutic agents targeting the BRCA pathway can be seen in FIG. 26, where co-administration of compound 10 and Olaparib (a PARP inhibitor) cause enhanced cell death relative to either alone.

PARP inhibitors can exhibit potent and selective activity against BRCA1 and BRCA2 mutated breast, ovarian, and other cancers. The use of a RAD51-disrupting compound can mimic the effects of a BRCA2 mutation and can potentially render a wider array of cancers treatable by PARP inhibitors. The PARP inhibitors can be, for example, olaparib, veliparib, niraparib, talazoparib, rucaparib, and CEP-9722.

A compound disclosed herein can be used in combination with other chemotherapeutic agents. The chemotherapeutic agents can include, for example, anti-PD1 agents, pembrolizumab, melphalan, doxorubicin, adrianmycin, etoposide, camptothecins, mitomycin C, cisplatin, oxaliplatin, carboplatin, or gemcitabine.

The compounds can be, for example, small molecules, biologics, antibodies, peptidomimetics, or peptides. The compound may be a peptide.

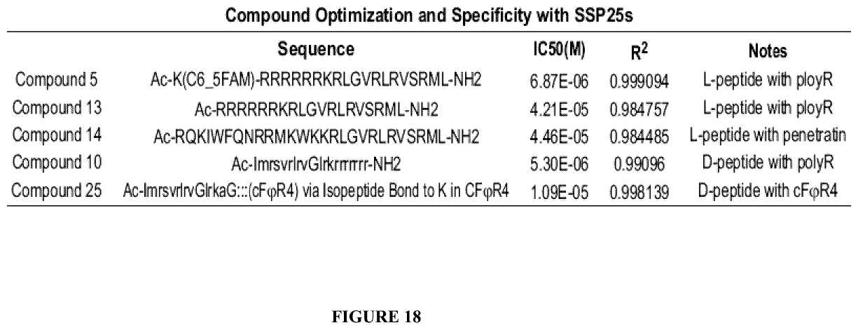

The compounds disclosed herein can harbor a cell penetration entity (CPP) or a protein transduction domain (PTD) to facilitate entry into the target cell. Protein transduction can refer to the delivery of peptides, proteins, and other molecules across cytoplasmic membranes into cells. The compounds can include at least one cell-penetration peptide (CPP) signal sequence. Examples of CPPs include HIV-TAT (GRKKRRQRRRPPQ; SEQ ID NO.: 13), R8 (RRRRRRRR; SEQ ID NO.: 14), MAP (KLALKLALKALKAALKLA; SEQ ID NO.: 15), transportan (GWTLNSAGYLLGKINLKALAALAKKIL; SEQ ID NO.: 16), pegelin (RGGRLSYSRRRFSTSTGR; SEQ ID NO.: 17), penetrin (RQIKIWFQNRRMKWKK; SEQ ID NO.: 18), cFΦR4 (cyclic heptapeptide cyclo (FØRrRrK) where Φ is 1-2-naphthylalanine linked via an isopeptide bond between the epsilon amino of the lysine to the carboxyl group of the terminal glycine in the peptide sequence, see Qian et al. Biochemistry. 2014 Jun. 24; 53 (24): 4034-46.), and derivatives or combinations thereof. A comparison of various compounds according to the invention with various cell-penetrating peptides can be seen in FIG. 18, wherein analogous sequences are tested with various different cell-penetrating peptides and demonstrate similar cellular activity against SSP25 cells.

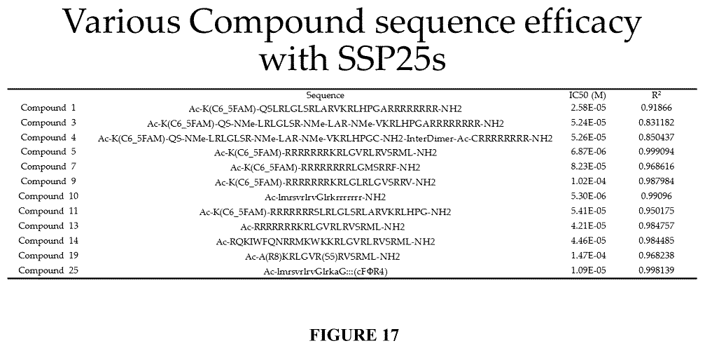

Any of the peptide compounds referred to herein can be N-terminally (e.g. alpha-amine) acetylated, C-terminally amidated, or backbone N-methylated. Any of the compounds referred to herein can be modified to include a 5FAM (Fluorescein amidite) label linked via a 6 carbon chain to the epsilon amino group of a lysine residue. Examples of compounds according to the invention that encompass various amino acid chemical modifications and display activity against SSP25 cells can be seen in FIG. 17.

The peptides disclosed herein can also be stabilized by conversion to peptidomimetic entities. A peptidomimetic can be a polymer encompassing amino acid residues joined together through amide bonds. Such stabilization approaches can include, for example, cyclization to macrocycles, lactam esterification, N-methylation of the backbone residues, hydrocarbon stapling, and combinations thereof.

The peptides disclosed herein can be stabilized or modified by conversion to “retro-inverso” entities. Retro-inverso peptides are linear peptides whose amino acid sequence is reversed and the alpha-carbon-center chirality of the amino acid subunits is inverted as well. Such a modification is known to increase stability of peptides when the original peptide is an (L)-peptide.

A compound of the invention can have, for example, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acid residues. In some embodiments, a compound of the invention is not an antibody. In some embodiments, a compound of the invention is an antibody or a functional binding fragment/derivative thereof (e.g. a Fab fragment or an ScFv). A compound of the invention can contain a RAD51 interacting motif with the following consensus sequence: R-L-G-L/M/V-S-R-R/L/K-R/F/V (SEQ ID NO.: 19).

A compound of the invention can comprise a sequence according to any one of the formulas in Table 3 below, or an invert thereof:

TABLE 3

| | |
|---|---|
| Formula 1 | [T/K/R/Q]₁₋₃-[L/I/V/F/M/W/Y]-[R/G/S]- |
| | [L/I/V/F/M/W/Y] (formula I) |
| | (SEQ ID NO.: 70) |
| Formula II | [R/K]-[L]-[G]-[M/V]-(formula II) |
| | (SEQ ID NO.: 71) |
| Formula III | [R]-[L]-[G]-[V]-[M/V]-[L/I/V/F/M/A/ |
| | W/Y] (formula III) |
| | (SEQ ID NO.: 72) |
| Formula IV | [T/K/R/Q]₁₋₂-[R]-[L]-[G]-[V]-[M/V]- |
| | [L/I/V/F/M/A/W/Y] (formula IV) |
| | (SEQ ID NO.: 73) |

Any of the polypeptides encompassed by formula I, II, III, or IV can include one or more, 2 or more, 3 or more, 4 or more, 5 or more, 6 or more non-naturally occurring amino acids or (D)-amino acids. In some embodiments, the entire peptide is composed of D-amino acids or non-naturally occurring amino acids.

Thus, in some embodiments, the present disclosure contemplates a compound comprising Formula I, II, III or IV, wherein any one or more of the amino acids are optionally non-natural amino acids or (D)-amino acids.

A compound of the invention can comprise an amino acid sequence according to any of the SEQ IDs in Table 4 below:

TABLE 4
Compounds and amino acid sequences they contain

| | | |
|---|---|---|
| Compound 1 | SEQ ID NO.: 1 | KQSLRLGLSRLARVKRLHPGARRR |
| | | RRRRR |
| Compound 2 | SEQ ID NO.: 2 | KQSLRLGQSRLARVKRLHPGARRR |
| | | RRRRR |
| Compound 3 | SEQ ID NO.: 3 | KQSLRLGLSRLARVKRLHPGARRR |
| | | RRRRR |
| Compound 4 | SEQ ID NO.: 4 | KQSNLRLGLSRLARVKRLHPGCCR |
| | | RRRRRRR |
| Compound 5 | SEQ ID NO.: 5 | KRRRRRRRKRLGVRLRVSRML |
| Compound 6 | SEQ ID NO.: 6 | KRRRRRRRKRLGQRLRQSRML |
| Compound 7 | SEQ ID NO.: 7 | KRRRRRRRRLGMSRRF |
| Compound 9 | SEQ ID NO.: 8 | KRRRRRRRKRLGLRLGVSRRV |
| Compound 10 | SEQ ID NO.: 9 | LMRSVRLRVGLRKRRRRRRR |
| Compound 11 | SEQ ID NO.: 10 | KRRRRRRRSLRLGLSRLARVKRLH |
| | | PG |
| Compound 13 | SEQ ID NO.: 11 | RRRRRRRKRLGVRLRVSRML |
| Compound 14 | SEQ ID NO.: 12 | RQKIWFQNRRMKWKKRLGVRLRVS |
| | | RML |
| Compound 19 | SEQ ID NO.: 13 | A(R8)KRLGVR(S5)RVSRML |
| Compound 25 | SEQ ID NO.: 14 | LMRSVRLRVGLRKAG |

(R8) refers to the non-natural amino acid D-2-(7′-octenyl)alanine, and (S5) refers to the non-natural amino acid 2-(4′-pentenyl)alanine

Any of SEQ ID NO: 1-11 or 66-69 can include one or more, 2 or more, 3 or more, 4 or more, 5 or more, 6 or more non-naturally occurring amino acids or (D)-amino acids. In some embodiments, the entire peptide described in Table 4 is composed of (D)-amino acids or non-naturally occurring amino acids.

Exemplary compounds of the disclosure are provided below in Table 5:

TABLE 5

| | |
|---|---|
| Compound 1 | Ac-K(C6_5FAM)-QSLRLGLSRLARVKRLHPGARRRRRRRR-NH2 (SEQ ID NO: 85) |
| Compound 2 | Ac-K(C6_5FAM)-QSLRLGQSRLARVKRLHPGARRRRRRRR-NH2 (SEQ ID NO: 86) |
| Compound 4 | Ac-K(C6_5FAM)-QS-NMe-LRLGLSR-NMe-LAR-NMe-VKRLHPGC-NH2- |
| | InterDimer-Ac-CRRRRRRRR-NH2 (SEQ ID NOS 87 and 88) |
| Compound 5 | Ac-K(C6_5FAM)-RRRRRRRKRLGVRLRVSRML-NH2 (SEQ ID NO: 89) |
| Compound 6 | Ac-K(C6_5FAM)-RRRRRRRKRLGQRLRQSRML-NH2 (SEQ ID NO: 90) |
| Compound 7 | Ac-K(C6_5FAM)-RRRRRRRRLGMSRRF-NH2 (SEQ ID NO: 91) |
| Compound 9 | Ac-K(C6_5FAM)-RRRRRRRKRLGLRLGVSRRV-NH2 (SEQ ID NO: 92) |
| Compound 10 | Ac-lmrsvrlrvGlrkrrrrrrr-NH2 |
| Compound 11 | Ac-K(C6_5FAM)-RRRRRRRSLRLGLSRLARVKRLHPG-NH2 (SEQ ID NO: 93) |
| Compound 13 | Ac-RRRRRRRKRLGVRLRVSRML-NH2 (SEQ ID NO: 94) |
| Compound 14 | Ac-RQKIWFQNRRMKWKKRLGVRLRVSRML-NH2 (SEQ ID NO: 95) |
| Compound 19 | Ac-A(R8)KRLGVR(S5)RVSRML-NH2 (SEQ ID NO: 96) |
| Compound 25 | Ac-lmrsvrlrvGlrkaG:::(cFΦR4) |

K(C6_5FAM) −> refers to a 5FAM (Fluorescein amidite) label linked via a 6 carbon chain to the epsilon amino group of a lysine.
Ac −> refers to alpha amine acetylation
NH2 −> refers to amidation of the carboxy terminal
R8 and S5 −> refer to a i + 7 hydrocarbon staple between R8, which is D-2-(7′-octenyl)alanine, and S5, which is 2-(4′-pentenyl)alanine
cFΦR4 −> refers to cyclic heptapeptide cyclo(FΦRrRrK) where Φ is 1-2-naphthylalanine linked via an isopeptide bond between the epsilon amino of the lysine to the carboxyl group of the terminal glycine in the peptide sequence
For the purposes of Table 5, lower case letters indicate (D)-amino acids while upper
case letters indicate (L)-amino acids

The compounds disclosed herein can also include, for example, non-binding, negative control peptides that harbor point mutations. The negative control peptides would not disrupt the interaction between RAD51 and BRCA2, and can be used as experimental controls.

A compound disclosed herein can inhibit a protein-protein interaction by, for example, competitive or allosteric inhibition. A compound herein can bind a cellular target that is associated with, for example, the DNA damage repair pathway. The binding can cause a decrease in the deleterious effects of the mutated gene in the DNA damage repair pathway.

A compound disclosed herein can target the interaction between RAD51 and BRCA2 or RAD51AP1. The compound can inhibit the interaction between RAD51 and RAD51AP1.

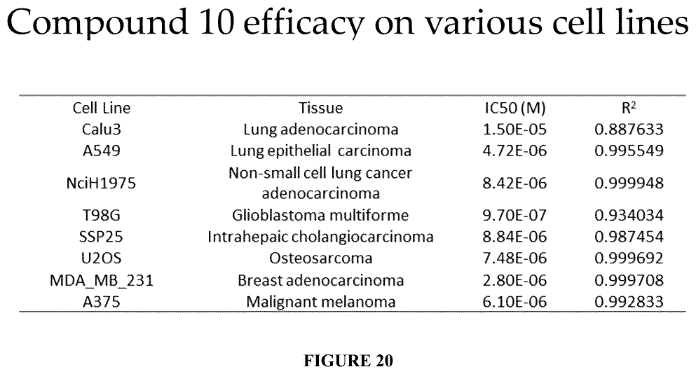

The compound can be tested on cell lines that harbor resistant mutations, are programmed to become resistant to drugs or apoptosis, or have mutations specific to the DNA damage repair pathway. Cell lines that can be tested in a method disclosed herein include, for example, HEK-293T, H1299, HCT-116, MCF-7, U2OS, U251, U87, T98G, human GBM, A549 NSCLC, H1993, H2073, MES-SA, MES-SA/Dx5, HT1080, HeLa, Saos-2, IMR90, SSP-25, PC3, LnCAP, Calu3, NciH1975, MDA_MB_231, A375, and mouse embryonic fibroblasts (MEFs). An example of this testing can be seen in FIG. 20, wherein compound 10 is tested on a panel of cell lines and displays IC50s in the 10\hat{ }−7 to 10\hat{ }−5 range, suggesting that a wide variety of cancer cells may be sensitive to RAD51 perturbation by compounds disclosed here.

A compound disclosed herein can bind to a subregion of human RAD51. Human RAD51 can comprise the sequence:

(SEQ ID NO.: 82)
MAMQMQLEANADTSVEEESFGPQPISRLEQCGINANDVKKLEEAGFHTVE
AVAYAPKKELINIKGISEAKADKILAEAAKLVPMGFTTATEFHQRRSEII
QITTGSKELDKLLQGGIETGSITEMFGEFRTGKTQICHTLAVTCQLPIDR
GGGEGKAMYIDTEGTFRPERLLAVAERYGLSGSDVLDNVAYARAFNTDHQ
TQLLYQASAMMVESRYALLIVDSATALYRTDYSGRGELSARQMHLARFLR
MLLRLADEFGVAVVITNQVVAQVDGAAMFAADPKKPIGGNIIAHASTTRL
YLRKGRGETRICKIYDSPCLPEAEAMFAINADGVGDAKD.

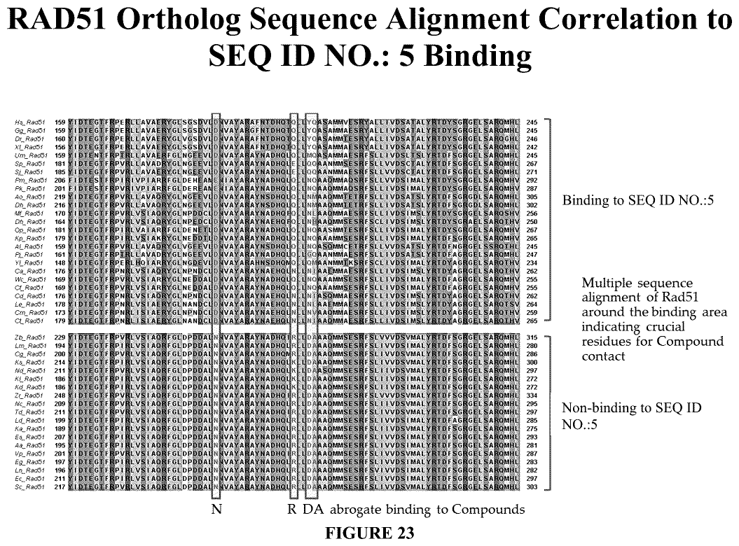

A compound as disclosed herein can also interact with a subregion of a sequence at least 80 or at least 90 percent identical to human RAD51 (e.g. SEQ ID NO.: 82). A compound as disclosed herein can bind to RAD51AP1's binding site on RAD51. A compound as disclosed herein can bind within a subregion of human RAD51 (e.g. SEQ ID NO.: 82), wherein the subregion is amino acids 190-339 of human RAD51 (e.g. SEQ ID NO.: 82). A compound as disclosed herein can interact with a subregion of human RAD51 (e.g. SEQ ID NO.: 82), wherein the subregion is amino acids 190-218 of human RAD51 (e.g. SEQ ID NO.: 82). A compound as disclosed herein can interact with at least one of residues 202, 205, and 206 of human RAD51 (e.g. SEQ ID NO.: 82). A compound as disclosed herein can interact with at least two of residues 202, 205, and 206 of human RAD51 (e.g. SEQ ID NO.: 82). A compound as disclosed herein can interact with all three of residues 202, 205, and 206 of human RAD51 (e.g. SEQ ID NO.: 82). A compound as disclosed herein can interact with residue 187 of human RAD51 (e.g. SEQ ID NO.: 82). A compound as disclosed herein can interact with at least one of residues 187, 202, 205, and 206 of human RAD51 (e.g. SEQ ID NO.: 82). A compound as disclosed herein can interact with at least two of residues 187, 202, 205, and 206 of human RAD51 (e.g. SEQ ID NO.: 82). A compound as disclosed herein can interact with at least three of residues 187, 202, 205, and 206 of human RAD51 (e.g. SEQ ID NO.: 82). A compound as disclosed herein can interact with all four of residues 187, 202, 205, and 206 of human RAD51 (e.g. SEQ ID NO.: 82). An illustration of these residues can be found in FIG. 23, where sequences alignments are clustered based on their ability to bind or not bind an exemplary peptide according to the invention (SEQ ID NO.: 5), and mutation of residues 187, 202, 205, and 206 correlates with binding ability.

### Amino Acids

Any of the compounds described herein can be include hydrophilic amino acids, hydrophobic amino acids, charged amino acids, uncharged amino acids, acidic amino acids, basic amino acids, neutral amino acids, aromatic amino acids, aliphatic amino acids, natural amino acids, non-natural amino acids, synthetic amino acids, artificial amino acids, capped amino acids, genetically-encoded amino acids, non-genetically encoded amino acids, and amino acid analogues, homologues, and congeners.

The peptides and polypeptides herein can include one or more D-amino acids or non-naturally occurring amino acids or all D-amino acids or non-naturally occurring amino acids. The peptides and polypeptides herein can be inverted and include one or more D-amino acids or non-naturally occurring amino acids, or all D-amino acids or non-naturally occurring amino acids.

Amino acids herein can be designated using their one letter or three letter abbreviation. IUPAC designation is provided below:

| Abbreviation | | Amino acid name |
|---|---|---|
| Ala | A | Alanine |
| Arg | R | Arginine |
| Asn | N | Asparagine |
| Asp | D | Aspartic acid (Aspartate) |
| Cys | C | Cysteine |
| Gln | Q | Glutamine |
| Glu | E | Glutamic acid (Glutamate) |
| Gly | G | Glycine |
| His | H | Histidine |
| Ile | I | Isoleucine |
| Leu | L | Leucine |
| Lys | K | Lysine |
| Met | M | Methionine |
| Phe | F | Phenylalanine |
| Pro | P | Proline |
| Ser | S | Serine |
| Thr | T | Threonine |
| Trp | W | Tryptophan |
| Tyr | Y | Tyrosine |
| Val | V | Valine |
| Asx | B | Aspartic acid or Asparagine |
| Glx | Z | Glutamine or Glutamic acid. |

A non-natural amino acid used in compounds described herein can be, for example, an amino acid that is prepared chemically or expressed by tRNA synthetase technology. A non-limiting example of an achiral amino acid that can be used in compounds described herein is glycine (G, Gly). Non-limiting examples of L-enantiomeric and D-enantiomeric amino acids that can be used in compounds described herein are: alanine (A,Ala); arginine (R, Arg); asparagine (N, Asn); aspartic acid (D, Asp); cysteine (C, Cys); glutamic acid (E, Glu); glutamine (Q, Gln); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); and valine (V, Val). In some embodiments, conservative or non-conservative substitutions of amino acids are possible of any compounds described herein.

Any of the compounds described herein can be modified by conservative amino acid substitution. Conservative amino acid substitutions involve the substitution of an amino acid with a chemically similar amino acid. Conservative substitution tables providing functionally similar amino acids are available from a variety of references (see, for e.g., Creighton, Proteins: Structures and Molecular Properties (W H Freeman & Co.; 2^{nd} edition (December 1993)). The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M)

Non-natural amino acids can be substituted for natural/canonical amino acids in any of the compounds described herein, particularly when the non-natural amino acids have similar chemical properties (e.g. hydrophobicity, hydrophilicity). Non-natural amino acids are amino acids that are not one of the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) or pyrolysine or selenocysteine; other terms that may be used synonymously with the term “non-natural amino acid” are “non-naturally encoded amino acid, “unnatural amino acid,” “non-naturally-occurring amino acid,” and variously hyphenated and non-hyphenated versions thereof. The term “non-natural amino acid” includes, but is not limited to, amino acids that occur naturally by modification of a naturally encoded amino acid (including but not limited to, the 20 common amino acids or pyrolysine and selenocysteine) but are not themselves incorporated into a growing polypeptide chain by the translation complex. Examples of naturally-occurring amino acids that are not naturally-encoded include, but are not limited to, N-acetylglucosaminyl-L-serine, N-acetylglucosaminyl-L-threonine, and O-phosphotyrosine.

Non-natural amino acids include amino acid analogs. Amino acid analogs are compounds that have the same basic chemical structure as a naturally occurring amino acid, i.e., a central carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid.

### Peptide Synthesis

Compounds as described herein can be synthetic peptides. Synthetic peptides were synthesized following standard solid-phase peptide synthesis protocols. The identity and purity of the peptides were confirmed and determined by RP-HPLC, MS/MS, and peptide content analysis. The trifluoroacetic acid (TFA) was exchanged for a non-toxic salt form (e.g. acetate or HCl) and the purity of the peptides was at least 95% before use in experiments.

### Pharmaceutically-Acceptable Salts

The invention provides the use of pharmaceutically-acceptable salts of any therapeutic compound disclosed herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Metal salts can arise from the addition of an inorganic base to a compound of the invention. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the invention. In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrrazole, pipyrrazole, imidazole, pyrazine, or pipyrazine.

In some embodiments, an ammonium salt is a triethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrrazole salt, a pipyrrazole salt, an imidazole salt, a pyrazine salt, or a pipyrazine salt.

Acid addition salts can arise from the addition of an acid to a compound of the invention. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a methanesulfonate (mesylate) salt, an ethanesulfonate salt, a benzenesulfonate salt, a p-toluenesulfonate salt, a citrate salt, an oxalate salt, or a maleate salt.

### Purity of Compounds

Any compound herein can be purified. A compound herein can be least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure.

In some embodiments, compounds of the invention can be applied topically to the skin, or a body cavity, for example, oral, vaginal, bladder, cranial, spinal, thoracic, or pelvic cavity of a subject. In some embodiments, the compounds of the invention can be applied to an accessible body cavity.

Compounds disclosed herein can increase cell death or inhibit cell growth in a cell by, for example, about 2-fold, about 3-fold, about 4-fold, about 5-fold, about 6-fold, about 7-fold, about 8-fold, about 9-fold, about 10-fold, about 11-fold, about 12-fold, about 13-fold, about 14-fold, about 15-fold, about 16-fold, about 17-fold, about 18-fold, about 19-fold, about 20-fold, about 25-fold, about 30-fold, about 35-fold, about 40-fold, about 45-fold, about 50-fold, about 55-fold, about 60-fold, about 65-fold, about 70-fold, about 75-fold, about 80-fold, about 85-fold, about 90-fold, about 95-fold, about 100-fold, about 110-fold, about 120-fold, about 130-fold, about 140-fold, about 150-fold, about 160-fold, about 170-fold, about 180-fold, about 190-fold, about 200-fold, about 250-fold, about 300-fold, about 350-fold, about 400-fold, about 450-fold, about 500-fold, about 550-fold, about 600-fold, about 650-fold, about 700-fold, about 750-fold, about 800-fold, about 850-fold, about 900-fold, about 950-fold, about 1000-fold, about 1500-fold, or about 2000-fold greater than when the cell is not exposed to the compound.

Compounds disclosed herein can increase free calcium concentration in a cell by, for example, about 2-fold, about 3-fold, about 4-fold, about 5-fold, about 6-fold, about 7-fold, about 8-fold, about 9-fold, about 10-fold, about 11-fold, about 12-fold, about 13-fold, about 14-fold, about 15-fold, about 16-fold, about 17-fold, about 18-fold, about 19-fold, or about 20-fold. Compounds disclosed herein can increase free calcium concentration in a cell by, for example, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, atleast about 15-fold, at least about 16-fold, at least about 17-fold, at least about 18-fold, at least about 19-fold, or at least about 20-fold.

Compounds disclosed herein can display GI₅₀ values that are, for example, about 0.1 nM, about 0.2 nM, about 0.3 nM, about 0.4 nM, about 0.5 nM, about 0.6 nM, about 0.7 nM, about 0.8 nM, about 0.9 nM, about 1 nM, about 1.5 nM, about 2 nM, about 2.5 nM, about 3 nM, about 3.5 nM, about 4 nM, about 4.5 nM, about 5 nM, about 5.5 nM, about 6 nM, about 6.5 nM, about 7 nM, about 7.5 nM, about 8 nM, about 8.5 nM, about 9 nM, about 9.5 nM, about 10 nM, about 15 nM, about 20 nM, about 25 nM, about 30 nM, about 35 nM, about 40 nM, about 45 nM, about 50 nM, about 60 nM, about 70 nM, about 80 nM, about 90 nM, about 100 nM, about 110 nM, about 120 nM, about 130 nM, about 140 nM, about 150 nM, about 200 nM, about 250 nM, about 300 nM, about 350 nM, about 400 nM, about 450 nM, about 500 nm, about 600 nM, about 700 nM, about 800 nM, about 900 nM, about 1 μM, about 1.5 μM, about 2 μM, about 2.5 μM, about 3 μM, about 3.5 μM, about 4 μM, about 4.5 μM, about 5 μM, about 6 μM, about 7 μM, about 8 μM, about 9 μM, about 10 μM, about 15 μM, about 20 μM, about 25 μM, about 30 μM, about 35 μM, about 40 μM, about 45 μM, about 50 μM, about 60 μM, about 70 μM, about 80 μM, about 90 μM, about 100 μM, about 150 μM, about 200 μM, about 300 M, about 400 μM, about 500 μM, about 600 μM, about 700 μM, about 800 μM, about 900 μM, or about 1 mM.

Compounds disclosed herein can be used to treat cancer in a subject. A compound disclosed herein can, for example, slow the proliferation of cancer cells, or kill cancer cells. Non-limiting examples of cancer that can be treated by a compound of the invention include: acute lymphoblastic leukemia, acute myeloid leukemia, adrenocortical carcinoma, AIDS-related cancers, AIDS-related lymphoma, anal cancer, appendix cancer, astrocytomas, basal cell carcinoma, bile duct cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas, Burkitt lymphoma, carcinoma of unknown primary origin, central nervous system lymphoma, cerebellar astrocytoma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma, germ cell tumors, gallbladder cancer, gastric cancer, gastrointestinal carcinoid tumor, gastrointestinal stromal tumor, gliomas, hairy cell leukemia, head and neck cancer, heart cancer, hepatocellular (liver) cancer, Hodgkin lymphoma, Hypopharyngeal cancer, intraocular melanoma, islet cell carcinoma, Kaposi sarcoma, kidney cancer, laryngeal cancer, lip and oral cavity cancer, liposarcoma, liver cancer, lung cancers, such as non-small cell and small cell lung cancer, lymphomas, leukemias, macroglobulinemia, malignant fibrous histiocytoma of bone/osteosarcoma, medulloblastoma, melanomas, mesothelioma, metastatic squamous neck cancer with occult primary, mouth cancer, multiple endocrine neoplasia syndrome, myelodysplastic syndromes, myeloid leukemia, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer, ovarian germ cell tumor, pancreatic cancer, pancreatic cancer islet cell, paranasal sinus and nasal cavity cancer, parathyroid cancer, penile cancer, pharyngeal cancer, pheochromocytoma, pineal astrocytoma, pineal germinoma, pituitary adenoma, pleuropulmonary blastoma, plasma cell neoplasia, primary central nervous system lymphoma, prostate cancer, rectal cancer, renal cell carcinoma, renal pelvis and ureter transitional cell cancer, retinoblastoma, rhabdomyosarcoma, salivary gland cancer, sarcomas, skin cancers, skin carcinoma merkel cell, small intestine cancer, soft tissue sarcoma, squamous cell carcinoma, stomach cancer, T-cell lymphoma, throat cancer, thymoma, thymic carcinoma, thyroid cancer, trophoblastic tumor (gestational), cancers of unknown primary site, urethral cancer, uterine sarcoma, vaginal cancer, vulvar cancer, Waldenström macroglobulinemia, and Wilms tumor.

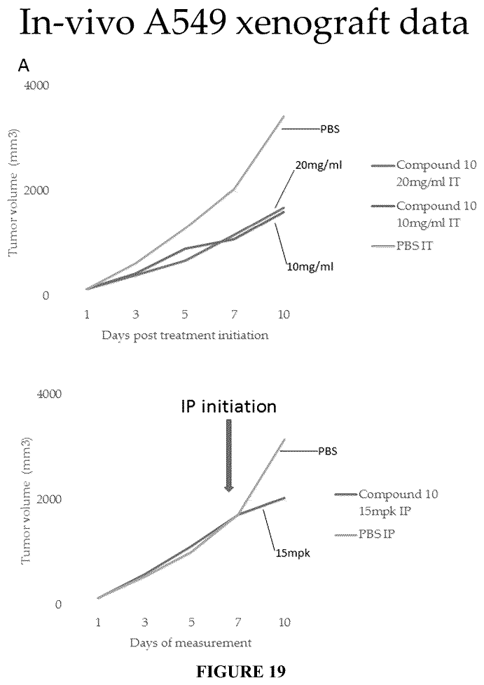

FIGS. 19 and 20 provide data showing suggestive cancer-related indications and administration methods for compounds according to the invention. FIG. 19 displays the activity of compound 10 in a xenograft model using A549 (a non-small cell lung cancer derived cell line), wherein compound 10 exhibits significant effects on tumor volume after only a few days of treatment. FIG. 20 shows the activity of compound 10 against various immortalized cell lines derived from cancer indications as described above, suggesting the efficacy of compounds related to 10 for various cancer indications.

Cholangiocarcinoma, with an incidence of about 1-2 cases per 100,000, is a rare cancer characterized by mutated epithelial cells, which originate in the bile ducts. Cholangiocarcinoma can be characterized as intrahepatic, perihilar, or distal bile duct cancer. Intrahepatic cholangiocarcinoma is a form of a cholangiocarcinoma that occurs within the bile ducts of the liver. The cancer in the bile duct can lead to the blockage of bile ducts and the accumulation of bilirubin. The major symptoms of cholangiocarcinoma include, for example, abnormal liver function tests, abdominal discomfort, jaundice, weight loss, pruritus, fever, loss of appetite, and changes in color of stool or urine.

Risk factors for cholangiocarcinoma include, for example, chronic inflammation or dysfunction of the bile ducts. Dysfunction of the bile ducts can manifest as, for example, primary sclerosing cholangitis, bile duct stones, choledochal cysts, liver fluke infections, polycystic liver disease, Caroli syndrome, cirrhosis, hepatitis B infection, or hepatitis C infection. Mutations in BRCA1/BRCA2 can also cause cholangiocarcinoma. Liver flukes are parasites that are commonly found in Asian countries in raw or poorly cooked fish. Other risk factors for cholangiocarcinoma include, for example, inflammatory bowel disease, age, obesity, exposure to thorium dioxide, diabetes, smoking, pancreatitis, HIV infection, asbestos exposure, or radon exposure.

Treatment for cholangiocarcinoma can include, for example, curative surgery, palliative surgery, laparoscopic procedures, external beam radiation therapy, three-dimensional conformal radiation therapy, intensity-modulated radiation therapy, stereotactic body radiotherapy, brachytherapy, 5-fluorouracil (5-FU), gemcitabine, cisplatin, capecitabine, or oxaliplatin.

In metastatic castration-resistant prostate cancer (CRPC), despite castrate levels of androgens, the androgen receptor (AR) remains active and drives cancer progression. The major symptoms of early stage prostate cancer include, for example, difficulty urinating, painful urination, frequent urination, hematuria, or pelvic pain. Prostate cancer often metastasizes to the bone and lymph nodes.

Hormone-dependent prostate cancer can become resistant to treatment after one to three years of therapy. Treatment for CRPC, includes, for example, anti-mitotic chemotherapeutics, docetaxel, cabazitaxel, bevacizumab, thalidomide, prednisone, sipuleucel-T, abiraterone, enzalutamide, or any combination thereof.

Pancreatic cancer arises when cells in the pancreas begin to multiply out of control and form a mass, which can metastasize to other parts of the body. The major symptoms of pancreatic cancer include, for example, upper abdominal pain, back pain, jaundice, loss of appetite, weight loss, and blood clots. Exocrine cancer can be, for example, pancreatic adenocarcinoma, acinar cell carcinoma, cystadenocarcinoma, pancreatoblastoma, adenosquamous carcinoma, signet ring cell carcinoma, hepatoid carcinoma, colloid carcinoma and pancreatic mucinous cystic neoplasm. Pancreatic neuroendocrine tumors (PanNETs) can arise elsewhere in the pancreas.

Treatment of pancreatic cancer can include, for example, surgical removal of the pancreas or the affected region of the pancreas, chemotherapy, 5-fluorouracil, gemcitabine, erlotinib, nab-paclitaxel, folic acid, irinotecan, oxaliplatin, FOLFIRINOX regimen, octreotide, lanreotide, everolimus, sunitinib, radiation therapy, or any combination thereof.

Small-cell carcinoma arises in the lungs, and can be highly malignant. Small-cell carcinoma is a neuroendocrine carcinoma that can exhibit aggressive behavior, rapid growth, early spread to distant sites, sensitivity to chemotherapy and radiation, and frequent association with distinct paraneoplastic syndromes, including, for example, hypercalcemia, Eaton-lambert syndrome, or syndrome of inappropriate diuretic hormone. Symptoms of small-cell carcinoma can include, for example, cough, dyspnea, weight loss, and frailty. Treatment for small-cell carcinoma can include, for example, cyclophosphamide, cisplatin, doxorubicin, etoposide, vincristine, paclitaxel, radiation therapy, or any combination thereof.

Compounds disclosed herein can be used to treat Bloom's syndrome in a subject. Bloom's syndrome is a rare autosomal recessive genetic disorder caused by a mutation in the BLM gene, which encodes for a DNA helicase enzyme. Cells for subjects afflicted with Bloom's syndrome exhibit marked chromosomal instability leading to increased sensitivity to UV radiation and higher risk for cancer. The features of Bloom's syndrome include, for example, unusually small stature, sparse fat tissue, high-pitched voice, long and narrow face, prominent nose, prominent ears, sun sensitivity, skin rash upon exposure to the sun, hypopigmentation, hyperpigmentation, reduced fertility in women, infertility in men, increased risk for diabetes, and chronic obstructive pulmonary disease, mild immune system abnormalities, and a shortened life expectancy.

Subjects can be, for example, elderly adults, adults, adolescents, pre-adolescents, children, toddlers, infants, and non-human animals. In some embodiments, a subject is a patient.

### Pharmaceutical Compositions

A pharmaceutical composition disclosed herein can be used, for example, before, during, or after treatment of a subject with another pharmaceutical agent.

A pharmaceutical composition disclosed herein can be a combination of any pharmaceutical compounds disclosed herein with other chemical components, such as carriers, stabilizers, diluents, dispersing agents, suspending agents, thickening agents, and/or excipients. The pharmaceutical composition facilitates administration of the compound to an organism. Pharmaceutical compositions can be administered in therapeutically-effective amounts as pharmaceutical compositions by various forms and routes including, for example, intravenous, subcutaneous, intramuscular, oral, parenteral, ophthalmic, subcutaneous, transdermal, nasal, vaginal, and topical administration.

A pharmaceutical composition can be administered in a local manner, for example, via injection of the compound directly into an organ, optionally in a depot or sustained release formulation or implant. Pharmaceutical compositions can be provided in the form of a rapid release formulation, in the form of an extended release formulation, or in the form of an intermediate release formulation. A rapid release form can provide an immediate release. An extended release formulation can provide a controlled release or a sustained delayed release.

For oral administration, pharmaceutical compositions can be formulated by combining the active compounds with pharmaceutically-acceptable carriers or excipients. Such carriers can be used to formulate liquids, gels, syrups, elixirs, slurries, or suspensions, for oral ingestion by a subject. Non-limiting examples of solvents used in an oral dissolvable formulation can include water, ethanol, isopropanol, saline, physiological saline, DMSO, dimethylformamide, potassium phosphate buffer, phosphate buffer saline (PBS), sodium phosphate buffer, 4-2-hydroxyethyl-1-piperazineethanesulfonic acid buffer (HEPES), 3-(N-morpholino)propanesulfonic acid buffer (MOPS), piperazine-N,N′-bis(2-ethanesulfonic acid) buffer (PIPES), and saline sodium citrate buffer (SSC). Non-limiting examples of co-solvents used in an oral dissolvable formulation can include sucrose, urea, cremaphor, DMSO, and potassium phosphate buffer.

Pharmaceutical preparations can be formulated for intravenous administration. The pharmaceutical compositions can be in a form suitable for parenteral injection as a sterile suspension, solution or emulsion in oily or aqueous vehicles, and can contain formulatory agents such as suspending, stabilizing and/or dispersing agents. Pharmaceutical formulations for parenteral administration include aqueous solutions of the active compounds in water-soluble form. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. The suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

The active compounds can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives.

The compounds can also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, and PEG. In suppository forms of the compositions, a low-melting wax such as a mixture of fatty acid glycerides, optionally in combination with cocoa butter, can be melted.

In practicing the methods of treatment or use provided herein, therapeutically-effective amounts of the compounds disclosed herein are administered in pharmaceutical compositions to a subject having a disease or condition to be treated. In some embodiments, the subject is a mammal such as a human. A therapeutically-effective amount can vary widely depending on the severity of the disease, the age and relative health of the subject, the potency of the compounds used, and other factors. The compounds can be used singly or in combination with one or more therapeutic agents as components of mixtures.

Pharmaceutical compositions can be formulated using one or more physiologically-acceptable carriers comprising excipients and auxiliaries, which facilitate processing of the active compounds into preparations that can be used pharmaceutically. Formulation can be modified depending upon the route of administration chosen. Pharmaceutical compositions comprising a compound disclosed herein can be manufactured, for example, by mixing, dissolving, emulsifying, encapsulating, entrapping, or compression processes.

The pharmaceutical compositions can include at least one pharmaceutically-acceptable carrier, diluent, or excipient and compounds described herein as free-base or pharmaceutically-acceptable salt form. Pharmaceutical compositions can contain solubilizers, stabilizers, tonicity enhancing agents, buffers and preservatives.

Methods for the preparation of compositions comprising the compounds disclosed herein include formulating the compounds with one or more inert, pharmaceutically-acceptable excipients or carriers to form a solid, semi-solid, or liquid composition. Solid compositions include, for example, powders, tablets, dispersible granules, capsules, and cachets. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use in the invention include liquid, powder, gel, nanosuspension, nanoparticle, microgel, aqueous or oily suspensions, emulsion, and any combination thereof.

Non-limiting examples of pharmaceutically-acceptable excipients suitable for use in the invention include binding agents, disintegrating agents, anti-adherents, anti-static agents, surfactants, anti-oxidants, coating agents, coloring agents, plasticizers, preservatives, suspending agents, emulsifying agents, anti-microbial agents, spheronization agents, and any combination thereof.

A composition can be, for example, an immediate release form or a controlled release formulation. An immediate release formulation can be formulated to allow the compounds to act rapidly. Non-limiting examples of immediate release formulations include readily dissolvable formulations. A controlled release formulation can be a pharmaceutical formulation that has been adapted such that release rates and release profiles of the active agent can be matched to physiological and chronotherapeutic requirements or, alternatively, has been formulated to effect release of an active agent at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

In some, a controlled release formulation is a delayed release form. A delayed release form can be formulated to delay a compound's action for an extended period of time. A delayed release form can be formulated to delay the release of an effective dose of one or more compounds, for example, for about 4, about 8, about 12, about 16, or about 24 hours.

A controlled release formulation can be a sustained release form. A sustained release form can be formulated to sustain, for example, the compound's action over an extended period of time. A sustained release form can be formulated to provide an effective dose of any compound disclosed herein (e.g., provide a physiologically-effective blood profile) over about 4, about 8, about 12, about 16 or about 24 hours.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

Multiple therapeutic agents can be administered in any order or simultaneously. In some embodiments, a compound of the invention is administered in combination with, before, or after an antibiotic. If simultaneously, the multiple therapeutic agents can be provided in a single, unified form, or in multiple forms, for example, as multiple separate pills. The agents can be packed together or separately, in a single package or in a plurality of packages. One or all of the therapeutic agents can be given in multiple doses. If not simultaneous, the timing between the multiple doses can vary to as much as about a month.

Therapeutic agents disclosed herein can be administered before, during, or after the occurrence of a disease or condition, and the timing of administering the composition containing a therapeutic agent can vary. For example, the compositions can be used as a prophylactic and can be administered continuously to subjects with a propensity to conditions or diseases in order to lessen a likelihood of the occurrence of the disease or condition. The compositions can be administered to a subject during or as soon as possible after the onset of the symptoms. The administration of the therapeutic agents can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein. A therapeutic agent can be administered as soon as is practicable after the onset of a disease or condition is detected or suspected, and for a length of time necessary for the treatment of the disease, such as, for example, from about 1 month to about 3 months. The length of treatment can vary for each subject.

Pharmaceutical compositions disclosed herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are packaged injectables, vials, or ampoules. Aqueous suspension compositions can be packaged in single-dose non-reclosable containers. Multiple-dose reclosable containers can be used, for example, in combination with or without a preservative. Formulations for injection can be presented in unit dosage form, for example, in ampoules, or in multi-dose containers with a preservative.

Pharmaceutical compositions provided herein, can be administered in conjunction with other therapies, for example, chemotherapy, radiation, surgery, anti-inflammatory agents, and selected vitamins. The other agents can be administered prior to, after, or concomitantly with the pharmaceutical compositions.

Depending on the intended mode of administration, the pharmaceutical compositions can be in the form of solid, semi-solid or liquid dosage forms, such as, for example, tablets, suppositories, pills, capsules, powders, liquids, suspensions, lotions, creams, or gels, for example, in unit dosage form suitable for single administration of a precise dosage.

For solid compositions, nontoxic solid carriers include, for example, pharmaceutical grades of mannitol, lactose, starch, magnesium stearate, sodium saccharin, talc, cellulose, glucose, sucrose, and magnesium carbonate.

Non-limiting examples of pharmaceutically active agents suitable for combination with compositions of the disclosure include anti-infectives, i.e., aminoglycosides, antiviral agents, antimicrobials, anticholinergics/antispasmotics, antidiabetic agents, antihypertensive agents, antineoplastics, cardiovascular agents, central nervous system agents, coagulation modifiers, hormones, immunologic agents, immunosuppressive agents, and ophthalmic preparations.

Compounds can be delivered via liposomal technology. The use of liposomes as drug carriers can increase the therapeutic index of the compounds. Liposomes are composed of natural phospholipids, and can contain mixed lipid chains with surfactant properties (e.g., egg phosphatidylethanolamine). A liposome design can employ surface ligands for attaching to unhealthy tissue. Non-limiting examples of liposomes include the multilamellar vesicle (MLV), the small unilamellar vesicle (SUV), and the large unilamellar vesicle (LUV). Liposomal physicochemical properties can be modulated to optimize penetration through biological barriers and retention at the site of administration, and to reduce a likelihood of developing premature degradation and toxicity to non-target tissues. Optimal liposomal properties depend on the administration route: large-sized liposomes show good retention upon local injection, small-sized liposomes are better suited to achieve passive targeting. PEGylation reduces the uptake of the liposomes by the liver and spleen, and increases the circulation time, resulting in increased localization at the inflamed site due to the enhanced permeability and retention (EPR) effect. Additionally, liposomal surfaces can be modified to achieve selective delivery of the encapsulated drug to specific target cells. Non-limiting examples of targeting ligands include monoclonal antibodies, vitamins, peptides, and polysaccharides specific for receptors concentrated on the surface of cells associated with the disease.

Non-limiting examples of dosage forms suitable for use in the disclosure include liquid, elixir, nanosuspension, aqueous or oily suspensions, drops, syrups, and any combination thereof. Non-limiting examples of pharmaceutically-acceptable excipients suitable for use in the disclosure include granulating agents, binding agents, lubricating agents, disintegrating agents, sweetening agents, glidants, anti-adherents, anti-static agents, surfactants, anti-oxidants, gums, coating agents, coloring agents, flavoring agents, coating agents, plasticizers, preservatives, suspending agents, emulsifying agents, plant cellulosic material and spheronization agents, and any combination thereof.

Compositions of the invention can be packaged as a kit. In some embodiments, a kit includes written instructions on the administration/use of the composition. The written material can be, for example, a label. The written material can suggest conditions methods of administration. The instructions provide the subject and the supervising physician with the best guidance for achieving the optimal clinical outcome from the administration of the therapy. The written material can be a label. In some embodiments, the label can be approved by a regulatory agency, for example the U.S. Food and Drug Administration (FDA), the European Medicines Agency (EMA), or other regulatory agencies.

### Dosing

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are liquids in vials or ampoules. Aqueous suspension compositions can be packaged in single-dose non-reclosable containers. Multiple-dose reclosable containers can be used, for example, in combination with a preservative. Formulations for parenteral injection can be presented in unit dosage form, for example, in ampoules, or in multi-dose containers with a preservative.

A compound disclosed herein can be present in a composition in a range of from about 1 mg to about 2000 mg; from about 100 mg to about 2000 mg; from about 10 mg to about 2000 mg; from about 5 mg to about 1000 mg, from about 10 mg to about 500 mg, from about 50 mg to about 250 mg, from about 100 mg to about 200 mg, from about 1 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 550 mg, from about 550 mg to about 600 mg, from about 600 mg to about 650 mg, from about 650 mg to about 700 mg, from about 700 mg to about 750 mg, from about 750 mg to about 800 mg, from about 800 mg to about 850 mg, from about 850 mg to about 900 mg, from about 900 mg to about 950 mg, or from about 950 mg to about 1000 mg.

A compound disclosed herein can be present in a composition in an amount of about 1 μg, about 10 μg, about 100 μg, about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg.

A therapeutically-effective amount of the compound can be, for example, about 0.1 nM, about 0.2 nM, about 0.3 nM, about 0.4 nM, about 0.5 nM, about 0.6 nM, about 0.7 nM, about 0.8 nM, about 0.9 nM, about 1 nM, about 1.5 nM, about 2 nM, about 2.5 nM, about 3 nM, about 3.5 nM, about 4 nM, about 4.5 nM, about 5 nM, about 5.5 nM, about 6 nM, about 6.5 nM, about 7 nM, about 7.5 nM, about 8 nM, about 8.5 nM, about 9 nM, about 9.5 nM, about 10 nM, about 15 nM, about 20 nM, about 25 nM, about 30 nM, about 35 nM, about 40 nM, about 45 nM, about 50 nM, about 60 nM, about 70 nM, about 80 nM, about 90 nM, about 100 nM, about 110 nM, about 120 nM, about 130 nM, about 140 nM, about 150 nM, about 200 nM, about 250 nM, about 300 nM, about 350 nM, about 400 nM, about 450 nM, about 500 nm, about 600 nM, about 700 nM, about 800 nM, about 900 nM, about 1 μM, about 1.5 μM, about 2 μM, about 2.5 μM, about 3 μM, about 3.5 μM, about 4 μM, about 4.5 μM, about 5 μM, about 6 μM, about 7 μM, about 8 μM, about 9 μM, about 10 μM, about 15 μM, about 20 μM, about 25 μM, about 30 μM, about 35 μM, about 40 μM, about 45 μM, about 50 μM, about 60 μM, about 70 μM, about 80 μM, about 90 μM, about 100 μM, about 150 μM, about 200 μM, about 300 μM, about 400 μM, about 500 μM, about 600 μM, about 700 μM, about 800 μM, about 900 μM, or about 1 mM.

In some embodiments, a dose can be expressed in terms of an amount of the drug divided by the mass of the subject, for example, milligrams of drug per kilograms of subject body mass. In some embodiments, a compound is administered in an amount ranging from about 5 mg/kg to about 50 mg/kg, 250 mg/kg to about 2000 mg/kg, about 10 mg/kg to about 800 mg/kg, about 50 mg/kg to about 400 mg/kg, about 100 mg/kg to about 300 mg/kg, or about 150 mg/kg to about 200 mg/kg.

### EXAMPLES

### Example 1: Method for Identifying Protein-Protein Interaction Disruptor

To achieve stabilization of short peptides the terminator sequence and untranslated region (UTR) or a short protein (such as sORF1) was used. The N-end rule of protein stability to begin the peptide sequence with residues that minimize proteolysis (such as Met, Gly, Ala, Ser, Thr, Val, or Pro) was used.

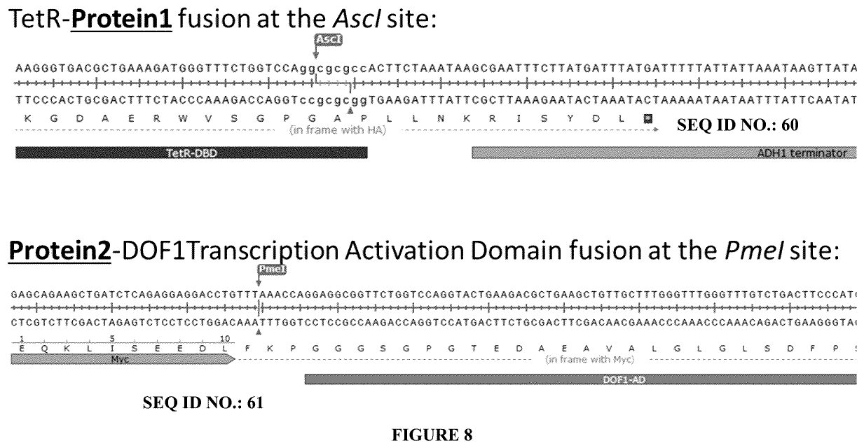

The PPI integration plasmid (Plasmid 1; FIG. 7) contained two restriction sites to enable the integration of two proteins that constituted the PPI of interest. The plasmid encoded for the fusion of an AD (such as Dof1; FIG. 8 (bottom)) and a DBD (such as TetR; FIG. 8 (top)) to each protein driven by either a strong promoter and terminator (such as ADH1), or by an inducible promoter (such as GAL1). Each protein fusion sequence was tagged with either FLAG or HA. The plasmid further included bacterial selection and propagation markers (i.e. ori and AmpR), and yeast replication and selection markers (i.e. TRP1 and CEN or 2 um). The plasmid also had sites for integration into the genome at a specified locus.

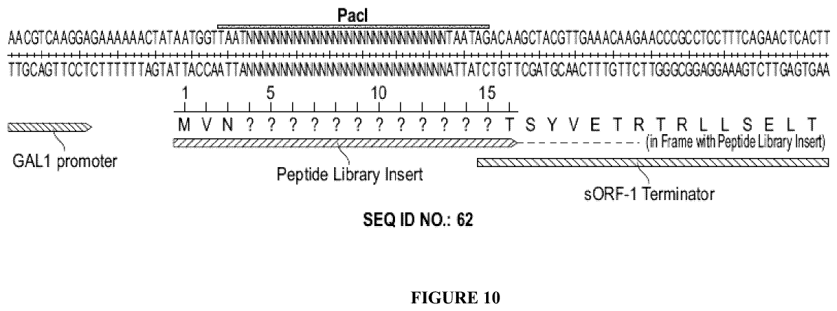

The selection and library plasmid (FIG. 9) included a restriction site for integration of a randomized peptide library (i.e. randomized NNK 60-mer sequences) driven by a strong promoter (such as the ADH1 promoter) or an inducible promoter (such as the GAL1 promoter). The initiation sequence of the selection and library plasmid was a fixed sequence of Methionine Valine Asparagine (MVN) to maximize the half-life of the peptide, and terminated with the UTR of a short protein (sORF1); (FIG. 10; SEQ ID NO.: 62). The peptide was also tagged with a Myc tag.

The selection and library plasmid additionally encoded for one or more of the agents outlined as ‘death agents’ (e.g. cytotoxic reporters) driven by a promoter, which depends on the DBD present in the PPI integration plasmid (FIG. 7). The TetO sequence in the selection and library plasmid can become bound by TetR in the PPI integration plasmid. The random library was inserted at the PacI site right in between the GAL1 promoter and the sORF-1 terminator.

To ensure repression of the ‘death agents,’ the selection and library plasmid included a silencing construct, TetR′-Tup11 fusion driven by a strong promoter (ADH1) to bind the DBD and silence transcription in the presence of doxycycline.

The selection and library plasmid further included bacterial selection and propagation markers (ori and AmpR), and yeast replication and selection markers (LEU2 and CEN or 2 um) (FIG. 9).

The confirmation plasmid (FIG. 11) was used to confirm expression of the reporters and the successful construction of the strains. It includes a direct fusion between the AD and DBD. The confirmation plasmid included bacterial selection and propagation markers (i.e. ori and AmpR), and yeast replication and selection markers (i.e. TRP1 and CEN or 2 um). The confirmation plasmid is used to confirm proper promoter integration in the background strain, for example, integration of TetO7 promoter before the ADE2 gene. Transformation with the confirmation plasmid allows for the activation of ADE2 only if the promoter is properly integrated.

The yeast background strain that was used for the experiment was Saccharomyces cerevisiae of either mating type or a diploid, and included the genomic integration of genetic reporters (such as ADE2, HIS3, and/or URA3) driven by the recognition sequence of the DBD used in the PPI Integration Plasmid (FIG. 7), such as TetO.

The background strain also expressed the enzymes necessary for the cyclization and methylation of peptides (e.g. lanthipeptides maturation enzymes from Lactococcus lactis (LanB, LanC, LanM, LanP), patellamide biosynthesis factors from cyanobacteria (PatD, PatG), butelase 1 from Clitoria ternatea, and GmPOPB from Galerina marginata).

The screen was performed using two methods. In the first method, the parental strain was propagated on selection media to ensure the presence of the PPI Integration plasmid, and that a faithful protein-protein interaction occurred, which was confirmed via use of the confirmation plasmid. The strain was cultured on media lacking Trp to ensure selection of colonies where the desired interaction occurred. The strain was then transformed with the selection and library plasmid, and immediately plated on selection media to ensure all components are present (i.e. on media lacking Trp and Leu) and inducing expression of any inducible component (i.e. with Gal).

In the second method, the strains were mated in batch to result in a diploid strain, which carried all the markers, the PPI, the ‘death agents,’ and the peptide. This batch culture was then propagated on solid medium, which enabled selection of all the system components (i.e. media lacking Leu and Trp) and induced expression of any inducible component (i.e. with Gal).

Surviving colonies from either option described above constituted colonies with a PPI that had been disrupted by a peptide and no longer triggered the death cascade triggered by the encoded ‘death agents’.

The peptide sequence that was able to disrupt the PPI was obtained by DNA sequencing the peptide-encoding region of the selection and library plasmid in each surviving colony.

To confirm specificity, the inducible marker was used to inactivate the production of either the PPI or the peptide and confirm specificity. For example, observation of cell survival only on media with Galactose wherein all the components are expressed, and no survival on media without Galactose when expression of the peptide is lost. The plasmid was then isolated and re-transformed into a fresh parental strain to confirm specificity.

Biochemical fractionation of the viable strain that contained the PPI, peptide and ‘death agent’, followed by pull-down experiments confirmed an interaction between the peptide sequence and either PPI partner using the encoded tags.

### Example 2: Treatment of SSP-25 Cells (Cholangiocarcinoma) With Compounds Disclosed Herein

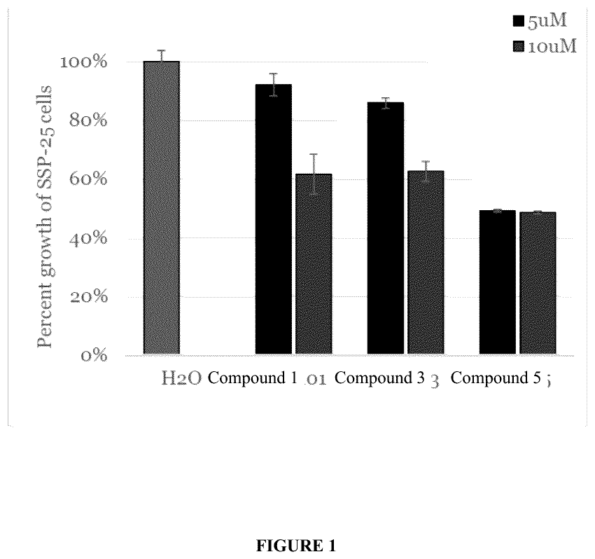

To assess the growth inhibitory properties of compounds disclosed herein, 5 μM or 10 μM of compounds 1, 3, and 5 were tested against a water mock treatment for growth inhibition of SSP-25 cells using an MTT assay after 48 hours of cell growth. FIG. 1 shows that compounds 1 and 3 had a dose-dependent effect on the growth of SSP-25, while compound 5 had the strongest inhibitory effect, regardless of concentration.

FIG. 13 shows the results of compound 5 treatment displaying acute cell death kinetics in SSP-25 cells as measured by an xCELLigence™ instrument. First, 5,000 SSP-25 cells were plated, and adherence and growth was measured for 20 hours using live measurements of current impedance through the plate using the xCELLigence™ instrument. At the 20 hour mark, 30 μM of compound 5 or a PBS control was added to the SSP-25 cells, which were cultured in 100 μM RPMI media with 10% FBS. Acute death kinetics were observed in real time and the cells were allowed to keep growing. At 72 hours, 30 μM more of compound 5 was added to both a pre-treated well and to a well previously treated with PBS only. FIG. 13 shows that resulting cell growth after initial treatment was not due to resistance, but to insufficient dosing of the compound. Further, singly-dosed cells continued to recover, which indicated reduced general cytotoxicity of the compound in the media or the lysed cells. This result supported the idea that an acute treatment with a dose above a certain threshold can be needed to activate the rapid killing mechanism due to increased calcium concentration. Additionally, treatment with compound 5 of previously untreated cells grown in PBS after 72 hours demonstrated the efficacy of compound 5 in acidified media.

### Example 3: Treatment of PC3 Cells (mCRPC Cells) With Compounds Disclosed Herein

FIG. 14 shows the results of compound 5 treatment displaying acute cell death kinetics in mCRPC cells as measured by an xCELLigence™ instrument. First, 5,000 PC3 cells were plated, and adherence and growth was measured for 24 hours using live measurements of current impedance through the plate using the xCELLigence™ instrument. At the 24 hour mark, 60 μM of compound 5, 60 μM of compound 6, or a PBS control was added to the PC3 cells grown in 100 μM DMEM with 10% FBS. Acute death kinetics were observed in real time and the cells were allowed to keep growing. At 84 hours, 100 μM more of compound 5 was added to the pre-treated cells. This was repeated again at the 124 hour mark. This regimen was successful in eliminating over 75% of the PC3 cells compared to the compound 6 and PBS controls.

FIG. 14 showed that compound 5, but not compound 6, was highly potent against PC3 cells. Furthermore, increased dosing was more effective in killing PC3 cells in a rapid way, but at higher concentrations than required for killing of SSP-25 cells, indicating either altered compound uptake kinetics or altered threshold required to trigger the acute cell death due to increased intracellular calcium concentrations.

### Example 4: Selective Killing of Cells

To assess the specificity of compounds disclosed herein in targeting cancers with mutations in the BRCA2 pathway, compounds 1 and 5 were tested against HeLa (human cervical cancer) and SSP-25 cells (cholangiocarcinoma cell line) (FIG. 2). To measure killing of the cells by compounds 1 and 5, a cell stain (CytoTox Red™) was used, which can be measured spectrophotometrically, and is indicative of cell viability. First, 5,000 cells were incubated in 96-well culture plates overnight. The media (RPMI with 10% FBS) was removed and replaced with serum free media (RPMI) containing 125 nM CytoTox Red™. FITC-labeled compounds 5 and 1 were added to concentrations of 5 μM and 10 μM, respectively, and imaging commenced using an IncuCyte™ instrument using 15 minute time points. 2× serum-containing media was added after one hour incubation, and the cells were imaged every 2 hours. To detect CytoTox Red™ the following parameters were used: Excitation Wavelength: 585 nm; Passband: [565,605] nm and Emission Wavelength: 635 nm; Passband: [625,705] nm. To detect compounds 5 and 1 using the FITC label, the following parameters were used: Excitation Wavelength: 460 nm; Passband: [440,480] nm and Emission Wavelength: 524 nm; Passband: [504,544] nm was used.

FIG. 2 provides an image of the stained cells, which prior to treatment show little staining in both the HeLa (201) and SSP-25 (203) cell populations. After treatment with compounds 1 or 5, there was little cell death in the HeLa cells (202), while the compounds showed greatest efficacy in the SSP-25 cells as compared to placebo treatment with PBS (204), as indicated by the lighter gray staining of the cells (204). Both the HeLa and SSP-25 cells showed efficient uptake of the compounds as indicated by the light gray staining of the cells.

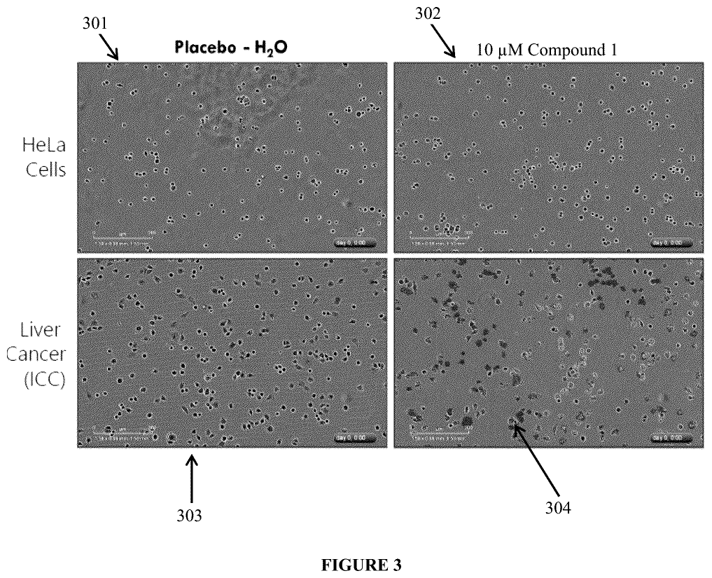

The same viability assay as above was repeated using 10 μM of compound 1. FIG. 3 displays the results of the experiment, and shows that compound 1 was specific to the SSP-25 cells as compared to a placebo treatment of PBS.

FIG. 3 provides an image of the stained cells, which prior to treatment show little staining in both the HeLa (301) and SSP-25 (302) cell populations. After treatment with compound 1, there is little cell death in the HeLa cells (303), while the liver cancer cells showed show the greatest staining (304), as indicated by the dark gray staining (304).

### Example 5: Specificity of the Compounds to RAD51

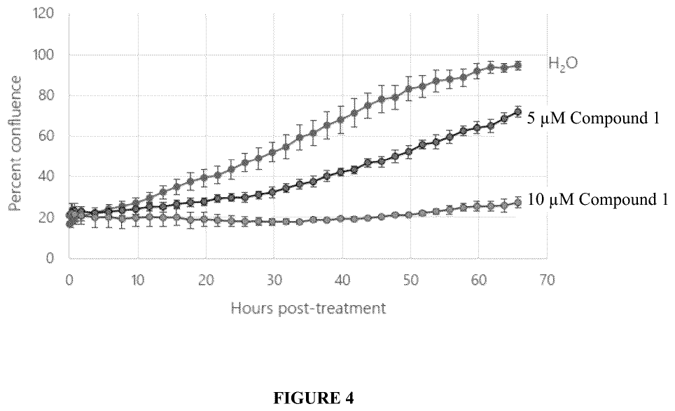

A growth assay was performed using SSP-25 cells with 5 μM or 10 μM of compound 1. FIG. 4 shows that treatment with compound decreased the percent confluence of the cells over the tested time period as compared to treatment with PBS. The experimental protocol described in Example 3 was used for the growth assay.

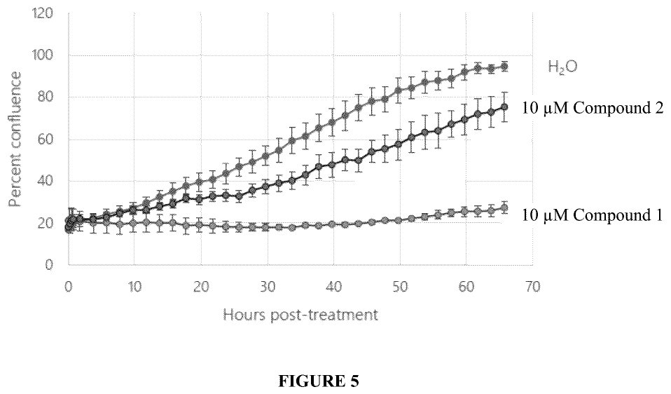

The same growth assay was repeated using 10 μM of compound 1 and compound 2. Compound 2 contains a single substitution within the core consensus sequence, which interrupts the binding of compound 2 to RAD51. Thus, as shown in FIG. 5, compound 2 was not able to decrease the growth of the SSP-25 cells to the same extent as compound 1, indicating the specificity of compound 1 to RAD51.

### Example 6: Counteracting Increased Sensitivity to Stress in Bloom's Cells

To assess the ability of compounds disclosed herein to rescue the drug-sensitivity of Bloom's Syndrome patient cells, GM08505, Blm cells were treated with PBS, 5 μM etoposide, or 5 μM etoposide and 10 μM compound 5. FIG. 6 shows that the growth of the Blm cells was affected by 5 μM etoposide treatment. However, treatment with PBS or the combination of etoposide and compound 5 was comparable, indicating suppression of cell growth defects induced by etoposide using compound 5 as measured by the percent confluence of the cells during the time period tested.

### Example 7: General Peptide Preparation and Optimization for Cellular Activity

Synthetic peptide compounds (compounds 1, 3, 4, 5, 7, 9, 10, 11, 13, 14, 19, and 25; see Table 5 and FIG. 17) were prepared with a diversity of modifications, amino acid stereoisomers, non-natural amino acids, and cell-penetrating peptides according to the consensus RAD51 binding sequence identified by the method of Example 1. Peptides were synthesized following standard solid-phase peptide synthesis protocols. The identity and purity of the peptides were confirmed and determined by RP-HPLC, MS/MS, and peptide content analysis. The trifluoroacetic acid (TFA) was exchanged for a non-toxic salt form (e.g. acetate or HCl) and the purity of the peptides was at least 95% before use in experiments.

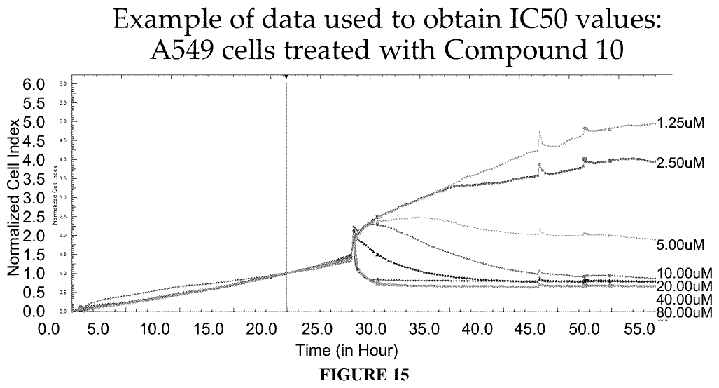

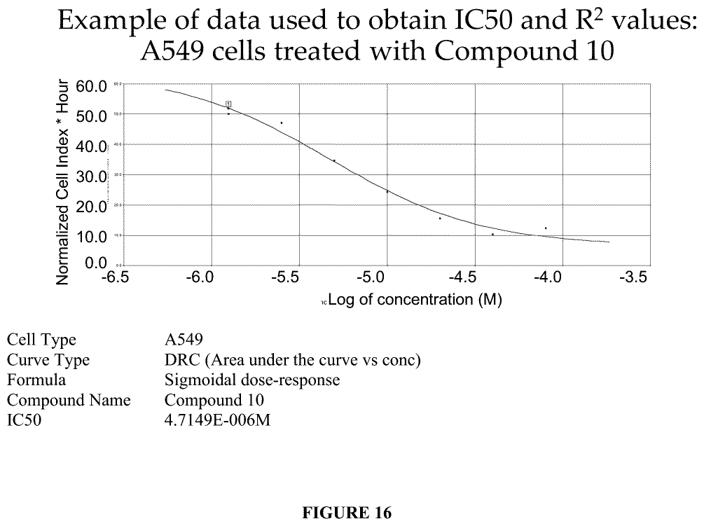

To optimize cellular activity, compounds were tested in xCELLigence cell death assays against various cell lines. An example of the workflow used is shown using A549 cells and compound 10 in FIGS. 15 and 16. FIG. 15 shows an exemplary xCELLigence treatment experiment, where the vertical line indicates the time of compound administration, and the cell index (Y-axis) represents cell viability. Data from xCELLigence traces at different compound concentrations from FIG. 15 was then used to generate a dose-response curve, which was then subjected to nonlinear regression using a sigmoidal dose-response model to generate IC50s and R2 values for cell death for each compound/cell line combination (FIG. 16).

An example of an optimization experiment is shown in FIG. 17, where compounds 1, 3, 4, 5, 7, 9, 10, 11, 13, 14, 19, and 25 were tested against SSP-25 cells. The cell death IC50s of the compounds varied from the 10⁻⁴ M to the 10⁻⁶ M range. An analysis of analogous peptides with different cell-penetrating peptides sequences incorporated into them (FIG. 18) demonstrated that the cellular activity is largely agnostic to which cell-penetrating peptide is chosen (as compounds 5, 13, 14, 10, and 25 all have similar IC50 values).

### Example 8: Mapping of Binding Site of RAD51-Binding Peptides Using Mutational Analysis in Y2H Assay

RAD51-binding peptides were tested for ability to bind RAD51 of several different species using a “conventional” Y2H assay, where interaction between the two-hybrid pair drove expression of ADE2 and HIS genes. In this Y2H assay the two-hybrid pair was RAD51 and the compound 5 peptide sequence (SEQ ID NO.: 5), so that cells having the interaction would survive (+) and cells not having the interaction would die (−) on selective media (−ADE, −HIS). While peptides successfully bound to H. sapiens, P. pastoris, and S. pombe RAD51, they did not bind to S. cerevisiae RAD51, indicating a sequence divergence between cerevisiae and human that precludes binding of the peptide. A subset of this data is summarized in Table 6.

TABLE 6
SEQ ID NO.: 5 binding to diverse species
orthologs of RAD51

| | Compound 5 |
|---|---|
| | Sequence |
| Species | Binding |
| H.sapiens Rad51 | + |
| G.gallus Rad51 | + |
| D.rerio Rad51 | + |
| S.pombe Rad51 | + |
| K.pastoris Rad51 | + |
| C.albicans Rad51 | + |
| K.lactis Rad51 | − |
| S.cerevisiae Rad51 | − |

Based on sequence comparisons of S. cerevisiae and H. sapiens RAD51, multiple cerevisiae-sapiens hybrid constructs of RAD51 were constructed and tested for their ability to bind RAD51-binding peptide in the same RAD51/SEQ ID NO.: 5 hybrid system described above. The RAD51 peptides only successfully bound a construct containing at least residues 190-339 of human RAD51, and did not bind a construct that included residues 218-339 of human RAD51, indicating the binding site lay somewhere in the 190-218 region. The data is summarized in Table 7.

TABLE 7
RAD51 peptide binding to RAD51 hybrid constructs

| | SEQ ID NO.: |
|---|---|
| Constructs | 5 Binding |
| S.cerevisiae Rad51 (1-226) + | + |
| H.sapiens Rad51 (168-339) | |
| S.cerevisiae Rad51 (1-250) + | + |
| H.sapiens Rad51 (190-339) | |
| S.cerevisiae Rad51 (1-278) + | − |
| H.sapiens Rad51 (218-339) | |

The ability of peptides to bind human RAD51 with various mutations was then tested by Y2H assay. The mutations encompassed both mutations described in the literature known to affect binding of RAD51 interaction partners such as BRCA2 and RAD51-AP1, and residues identified by a broader sequence alignment analysis of RAD51 species orthologs that do/do not bind SEQ ID NO.: 5 (FIG. 23, see residues “R”, “D” and “A” boxed). While mutations affecting BRCA2 binding had no effect on peptide binding, mutations that affected RAD51-AP1 binding did disrupt peptide binding. As these mutations were at residues 202, 205, and 206, they were consistent with the previous fusion data showing binding somewhere in the 190-218 region, and indicated the peptide SEQ ID NO.: 5 contacts at least residues 202, 205, and 206 of RAD51.

TABLE 8
RAD51 peptide binding to human
RAD51 bearing protein-
protein interaction-disrupting mutations

| | | | Rad51- | SEQ ID |
|---|---|---|---|---|
| Construct | BRCA2 | Rad51 | AP1 | NO.: 5 |
| Rad51 (F86E; A89E) | + | − | + | + |
| Rad51 (A190L; A192L) | − | − | + | + |
| Rad51 (S208L; A209E; | − | − | + | + |
| M210A) | | | | |
| Rad51 (S208E; A209D) | − | + | + | + |
| Rad51 (Q202R, Y205D, | + | + | − | − |
| Q206A) | | | | |

+ denotes a successful interaction between the two Y2H baits,
− denotes a lack of interaction
“BRCA2” denotes residues 1517-1551 of human BRCA2 used as Y2H bait.
“RAD51AP1” denotes residues 245-352 of human RAD51AP1 (GenBank AAH16330.1) used as Y2H bait.
“SEQ ID NO.: 5” denotes SEQ ID NO.: 5 used as Y2H bait.

It should be noted that the sequence alignment of FIG. 23 also indicates that residue 187 relative to human RAD51 is variant between orthologs that do/do not bind compounds, suggesting residue 187 of human RAD51 additionally plays a role in compound binding.

### Example 9: RAD51-Binding Peptide Interaction With Calcium Binding to RAD51

To test the interaction between calcium binding and peptide binding to RAD51, the Kds of active (Compound 5, 10) peptides for RAD51 were tested by a microscale thermophoresis assay, and the Kd of compound 5 was additionally performed in the presence of 1 mM calcium.

Microscale thermophoresis was performed as previously described (see, for e.g. Jerabek-Willemsen, M., Wienken, C. J., Braun, D., Baaske, P. & Duhr, S. Molecular interaction studies using microscale thermophoresis. Assay Drug Dev. Technol. 9, 342-353 (2011)). The affinity of the Compound was measured using the Monolith NT.115 from Nanotemper Technologies. Rad51 was fluorescently labelled according to the manufacturer's protocol and the labelled protein used for each assay was about 500 nM. A solution of unlabelled peptide was diluted for appropriate serial concentration gradient. The samples were loaded into silica capillaries (Polymicro Technologies). Measurements were performed at room temperature in buffer containing PBS, 0.1% Tween 20, 0.5 mM ATP, 2.5 μM ssDNA, with 1 mM CaCl2 (when mentioned). Measurements were done using 12% LED power and 60% MST power. The assays were repeated three times for each affinity measurement. Data analyses were performed using Nanotemper Analysis software and OriginPro 8.0 software provided by the manufacturer.

| Peptide | Buffer conditions | Kd (nM) |
|---|---|---|
| Compound 5 | PBS + 1 mM Ca²⁺ | 28.87 |
| Compound 5 | PBS | 8.37 |
| Compound 10 | PBS | 8.55 |

The microscale thermophoresis data is summarized in Table 9. Both active peptides (5 and 10) bound with a Kd approximately 8 nanomolar. However, when the in vitro concentration of calcium (Ca2+) in the RAD51 binding assay was increased to from 0 to 1 mM, the Kd increased 3.4-fold, indicating that there is allosteric communication between the calcium binding sites and the peptide binding interface on RAD51.

### Example 10: Compound Efficacy in an Athymic Xenograft Mouse Model of Cancer

Female athymic nu/nu mice (6-8 weeks old) were purchased from Simonsen labs (n=12) and were housed at the Murigenics vivarium in Vallejo. The mice were acclimated to the setting of the vivarium for 5 days and maintained on a standard chow diet; 12:12 dark/light cycle; and group housed (3 mice per cage) in hepa-filtered cages. After acclimation, each mouse was injected subcutaneously (lower left abdominal flank) with 5×10\hat{ }6 A549 (ATCC: CCL-185) cells mixed with 1:1 (v/v) with Matrigel solution. When the tumors reached a volume of ˜100 mm3, the mice were divided into separate treatment groups for the desired treatments. Experiments using both intra-tumoral (IT) and intraperitoneal (IP) administration were performed and the data are reported in FIG. 19.

### Experiment 1: Intra-Tumoral (IT) Dosing

Mice prepared as described above were divided into 4 groups:
Group 1: were not treated with anything (n=3)
Group 2: received an intra-tumoral injection of 50 microliters of Phosphate Buffered Saline (mock) (n=3)
Group 3: received an intra-tumoral injection of 50 microliters of 10 mg/ml compound 10 in Phosphate Buffered Saline (n=3)
Group 4: received an intra-tumoral injection of 50 microliters of 20 mg/ml compound 10 in Phosphate Buffered Saline (n=3)

The 50 microliter injections were carried out with a (30 g needle). The injections were administered on day zero and then again on day 3. The length (L) and the width (W) of the tumor mass was measured with a Vernier caliper and the tumor volume (V) was calculated as V=(L×W\hat{ }2)/2. The relative tumor volume of each tumor mass on each measurement day was normalized against the initial volume of the same mass on day zero right before initiation of injections. Measurements were performed on days 0, 3, and 6, and the data is summarized in FIG. 19, which shows measurable decreases in tumor size as early as day 3.

### Experiment 2: Intra-Peritoneal (IP) Dosing

Mice prepared as described above were divided into 2 groups:
Group 1: received an intra-peritoneal injection of 125 microliters of Phosphate Buffered Saline (mock) (n=2)
Group 2: received an intra-peritoneal injection of 125 microliters of 3 mg/ml compound 10 in Phosphate Buffered Saline (n=3)

The injections were carried out with a 30 g needle. The injections were administered on day seven. The length (L) and the width (W) of the tumor mass was measured with a Vernier caliper and the tumor volume (V) was calculated as V=(L×W\hat{ }2)/2. The relative tumor volume of each tumor mass on each measurement day was normalized against the initial volume of the same mass on day zero right before initiation of injections. Measurements were performed through days 0-10, with IP dosing on day 7, and the data is summarized in FIG. 19, which shows measurable decreases in tumor size as early as 3 days after injection (day 10).

### Example 11: Intracellular Ca2+ Chelation Suppresses the Cytotoxicity of Compound 10 in SSP-25 Cells

To further investigate the hypothesis that peptide binding to RAD51 in cells causes cell death via increases in intracellular free calcium levels, compound 10 alone and in combination with two different intracellular calcium chelators (1,2-Bis(2-aminophenoxy)ethane-N,N,N′,N′-tetraacetic acid tetrakis (acetoxymethyl ester) aka “BAPTA-AM”, and ammoniated ruthenium oxychloride aka “ruthenium red”) was tested in an xCELLigence™ cell death assay as in Example 3. The intracellular calcium chelators alone were also evaluated on the cells. In both cases, administration of calcium chelators rescues cell death, suggesting that the RAD51 binding compounds herein kill cells as a result of a rise in intracellular free Ca2+ ions, and that counteracting the rise in intracellular free Ca2+ ions prevents cell death due to the RAD51 binding compounds.

FIG. 23 demonstrates that addition of 10 μM BAPTA-AM to cells treated with 10 μM compound 10 rescues cell death relative to compound alone (see curves in FIG. 23, where the curve representing the BAPTA-AM+compound combination is roughly equivalent to PBS alone, while the curves representing compound 10 alone displays significant cell death).

FIG. 24 demonstrates that addition of 40 μM Ruthenium red to cells treated with 10 μM compound 10 rescues cell death relative to compound alone (see curves in FIG. 24, where the curve representing the ruthenium red+compound combination is roughly equivalent to PBS alone, while the curve representing compound alone displays significant cell death).

### Example 12: Olaparib Synergy With Compound 10 on SSP-25 Cells

To further investigate the hypothesis that peptide binding to RAD51 causes defective DNA repair and thus synergizes with other agents that target DNA repair, compound 10 or the poly ADP ribose polymerase (PARP) inhibitor Olaparib (AZD-2281, Lynparza, 4-[4-Fluoro-3-[(4-methoxypiperidin-1-yl)carbonyl]benzyl]phthalazin-1(2H)-one, 4-[[4-Fluoro-3-[(4-methoxy-1-piperidinyl)carbonyl]phenyl]methyl]-1(2H)-phthalazinone) alone or in combination were tested in an xCELLigence™ cell death assay as in Example 3.

FIG. 25 demonstrates that addition of 1 μM Olaparib to cells treated with 10 μM compound 10 enhances cell death relative to compound alone or Olaparib alone (see curves in FIG. 25, where the curve representing the olaparib+compound combination displays enhanced cell death relative to the other curves).

### Embodiments

The following non-limiting embodiments provide illustrative examples of the invention, but do not limit the scope of the invention.
Embodiment 1. A method of treating a condition, the method comprising administering to a subject in need thereof a therapeutically effective amount of a polypeptide that binds to a eukaryotic recombinase in a cell, wherein the binding of the polypeptide to the eukaryotic recombinase inhibits binding of the eukaryotic recombinase to a protein in the cell, wherein the inhibition of binding of the eukaryotic recombinase to the protein decreases or inhibits deoxyribonucleic acid (DNA) damage repair in the cell as compared to a cell that is not exposed to the polypeptide.
Embodiment 2. The method of embodiment 1, wherein the eukaryotic recombinase is RAD51.
Embodiment 3. The method of any one of embodiments 1-2, wherein the polypeptide is not an antibody.
Embodiment 4. The method of any one of embodiments 1-3, wherein the protein is BRCA2.
Embodiment 5. The method of any one of embodiments 1-3, wherein the protein is RAD51AP1.
Embodiment 6. The method of any one of embodiments 1-5, wherein the inhibition of binding of the eukaryotic recombinase to the protein occurs via competitive inhibition between the polypeptide and the protein.
Embodiment 7. The method of any one of embodiments 1-5, wherein the inhibition of binding of the eukaryotic recombinase to the protein occurs via allosteric inhibition of the binding of the eukaryotic recombinase to the protein.
Embodiment 8. The method of any one of embodiments 1-7, wherein the binding of the polypeptide to the eukaryotic recombinase in the cell decreases proliferation of the cell.
Embodiment 9. The method of any one of embodiments 1-7, wherein the binding of the polypeptide to the eukaryotic recombinase in the cell induces death of the cell.
Embodiment 10. The method of embodiment 9, wherein the cell death is apoptotic cell death.
Embodiment 11. The method of any one of embodiments 1-10, further comprising a decrease of homologous recombination in the cell.
Embodiment 12. The method of any one of embodiments 1-11, wherein the decrease or inhibition of DNA damage repair in the cell sensitizes the cell to a chemotherapeutic.
Embodiment 13. The method of any one of embodiments 1-12, wherein the polypeptide is from 4 to 70 amino acids in length.
Embodiment 14. The method of any one of embodiments 1-13, wherein the polypeptide is from 15 to 30 amino acids in length.
Embodiment 15. The method of any one of embodiments 1-14, wherein the polypeptide comprises a cell penetration peptide signal sequence.
Embodiment 16. The method of any one of embodiments 1-15, wherein the polypeptide comprises a RAD51 interacting motif, wherein the RAD51 interacting motif is SEQ ID NO.: 19.
Embodiment 17. The method of any one of embodiments 1-16, wherein the polypeptide comprises a protein transduction domain.
Embodiment 18. The method of any one of embodiments 1-17, wherein the polypeptide is SEQ ID NO.: 1.
Embodiment 19. The method of any one of embodiments 1-17, wherein the polypeptide is SEQ ID NO.: 3.
Embodiment 20. The method of any one of embodiments 1-17, wherein the polypeptide is SEQ ID NO.: 5.
Embodiment 21. The method of any one of embodiments 1-20, further comprising administration of a chemotherapeutic.
Embodiment 22. The method of embodiment 21, wherein the chemotherapeutic is an anti-PD1 agent.
Embodiment 23. The method of embodiment 21, wherein the chemotherapeutic is a PARP inhibitor.
Embodiment 24. The method of any one of embodiments 1-23, wherein the condition is cancer.
Embodiment 25. The method of any one of embodiments 1-24, wherein the condition is intrahepatic cholangiocarcinoma.
Embodiment 26. The method of any one of embodiments 1-24, wherein the condition is castration-resistant prostate cancer.
Embodiment 27. The method of any one of embodiments 1-23, wherein the condition is Bloom's Syndrome.
Embodiment 28. The method of any one of embodiments 1-27, wherein the administration is oral.
Embodiment 29. The method of any one of embodiments 1-27, wherein the administration is subcutaneous.
Embodiment 30. The method of any one of embodiments 1-27, wherein the administration is intravenous.
Embodiment 31. The method of any one of embodiments 1-30, wherein the therapeutically effective amount is from about 5 μM to about 1 mM.
Embodiment 32. The method of any one of embodiments 1-31, wherein the subject is human.
Embodiment 33. The method of any one of embodiments 1-32, wherein the cell exhibits an increase in calcium concentration upon binding of the polypeptide to the eukaryotic recombinase.
Embodiment 34. The method of embodiment 33, wherein the increase in calcium concentration induces death of the cell.
Embodiment 35. A method of inducing death in a cell, the method comprising contacting the cell with a polypeptide, wherein the polypeptide binds to a eukaryotic recombinase in the cell, wherein the binding of the polypeptide to the eukaryotic recombinase inhibits binding of the eukaryotic recombinase to a protein in the cell, wherein the inhibition of binding of the eukaryotic recombinase to the protein decreases or inhibits deoxyribonucleic acid (DNA) damage repair in the cell, wherein the inhibition of binding of the eukaryotic recombinase to the protein induces death in the cell as compared to a cell that is not contacted with the polypeptide.
Embodiment 36. A method of reducing drug resistance in a cell, the method comprising contacting the cell with a polypeptide, wherein the polypeptide binds to a eukaryotic recombinase in the cell, wherein the binding of the polypeptide to the eukaryotic recombinase inhibits binding of the eukaryotic recombinase to a protein in the cell, wherein the inhibition of binding of the eukaryotic recombinase to the protein decreases or inhibits deoxyribonucleic acid (DNA) damage repair in the cell, wherein the inhibition of binding of the eukaryotic recombinase reduces drug resistance of the cell as compared to a cell that is not contacted with the polypeptide.

1. A host cell configured to express:
(a) a first fusion protein comprising a first test protein and a DNA-binding moiety;
(b) a second fusion protein comprising a second test protein and a gene-activating moiety;
(c) a plurality of different cytotoxic reporters, wherein expression of each of the different cytotoxic reporters is under control of a DNA-binding sequence specific for the DNA-binding moiety; and
(d) an mRNA comprising a nucleotide sequence encoding a non-naturally occurring polypeptide,
wherein interaction between the first test protein and the second test protein causes the gene-activating moiety to activate expression of the plurality of different cytotoxic reporters.

2. The host cell of claim 1, wherein the host cell is eukaryotic.

3. The host cell of claim 1, wherein the host cell is fungal.

4. The host cell of claim 1, wherein the plurality of different cytotoxic reporters are selected from the group consisting of: a ribosomally-encoded xenobiotic agent, a ribosomally-encoded poison, and a ribosomally-encoded recombinase that excises an essential gene for viability.

5. The host cell of claim 1, wherein the plurality of different cytotoxic reporters are selected from the group consisting of: Cholera toxin, SpvB toxin, CARDS toxin, SpyA Toxin, HopU1, Chelt toxin, Certhrax toxin, EFV toxin, ExoT, CdtB, Diphtheria toxin, ExoU/VipB, HopPtoE, HopPtoF, HopPtoG, VopF, YopJ, AvrPtoB, SdbA, SidG, VpdA, Lpg0969, Lpg1978, YopE, SptP, SopE2, SopB/SigD, SipA, YpkA, YopM, Amatoxin, Phallacidin, Killer toxin KP1, Killer toxin KP6, Killer Toxin K1, Killer Toxin K28 (KHR), Killer Toxin K28 (KHS), Anthrax lethal factor endopeptidase, Shiga Toxin, and Ricin Toxin.

6. The host cell of claim 1, wherein the DNA binding moiety is a LexA, cI, Glucocorticoid reporter, TetR, or Ume6 DNA binding moiety.

7. The host cell of claim 1, wherein the gene activating moiety is a GAL4, B42, or Dof1 gene activating moiety.

8. The host cell of claim 1, wherein the non-naturally occurring polypeptide has 60 or fewer amino acids.

9. The host cell of claim 1, wherein the nucleotide sequence encoding the non-naturally occurring polypeptide further encodes an N-terminal sequence for peptide stabilization.

10. The host cell of claim 1, wherein the DNA binding moiety is a LexA, cI, Glucocorticoid reporter, TetR, or Ume6 DNA binding moiety, wherein the gene activating moiety is a GAL4, B42, or Dof1 gene activating moiety, and wherein the plurality of different cytotoxic reporters are selected from the group consisting of: a ribosomally-encoded xenobiotic agent, a ribosomally-encoded poison, and a ribosomally-encoded recombinase that excises an essential gene for viability.

11. A plurality of host cells, wherein each of the plurality of host cells is configured to express:
(a) a first fusion protein comprising a first test protein and a DNA-binding moiety;
(b) a second fusion protein comprising a second test protein and a gene activating moiety;
(c) a plurality of different cytotoxic reporters, wherein expression of each of the different cytotoxic reporters is under control of a DNA-binding sequence specific for the DNA-binding moiety; and
(d) an mRNA comprising a nucleotide sequence encoding a non-naturally occurring polypeptide,
wherein interaction between the first test protein and the second test protein causes the gene-activating moiety to activate expression of the plurality of different cytotoxic reporters.

12. The plurality of host cells of claim 11, wherein the plurality of host cells are eukaryotic.

13. The plurality of host cells of claim 11, wherein the plurality of host cells are fungal.

14. The plurality of host cells of claim 11, wherein the plurality of different cytotoxic reporters are selected from the group consisting of: a ribosomally-encoded xenobiotic agent, a ribosomally-encoded poison, and a ribosomally-encoded recombinase that excises an essential gene for viability.

15. The plurality of host cells of claim 11, wherein the plurality of different cytotoxic reporters are selected from the group consisting of: Cholera toxin, SpvB toxin, CARDS toxin, SpyA Toxin, HopU1, Chelt toxin, Certhrax toxin, EFV toxin, ExoT, CdtB, Diphtheria toxin, ExoU/VipB, HopPtoE, HopPtoF, HopPtoG, VopF, YopJ, AvrPtoB, SdbA, SidG, VpdA, Lpg0969, Lpg1978, YopE, SptP, SopE2, SopB/SigD, SipA, YpkA, YopM, Amatoxin, Phallacidin, Killer toxin KP1, Killer toxin KP6, Killer Toxin K1, Killer Toxin K28 (KHR), Killer Toxin K28 (KHS), Anthrax lethal factor endopeptidase, Shiga Toxin, and Ricin Toxin.

16. The plurality of host cells of claim 11, wherein the DNA binding moiety is a LexA, cI, Glucocorticoid reporter, TetR, or Ume6 DNA binding moiety.

17. The plurality of host cells of claim 11, wherein the gene activating moiety is a GAL4, B42, or Dof1 gene activating moiety.

18. The plurality of host cells of claim 11, wherein the non-naturally occurring polypeptide has 60 or fewer amino acids.

19. The plurality of host cells of claim 11, wherein the nucleotide sequence encoding the non-naturally occurring polypeptide further encodes an N-terminal sequence for peptide stabilization.

20. The plurality of host cells of claim 11, wherein the DNA binding moiety is a LexA, cI, Glucocorticoid reporter, TetR, or Ume6 DNA binding moiety, wherein the gene activating moiety is a GAL4, B42, or Dof1 gene activating moiety, and wherein the plurality of different cytotoxic reporters are selected from the group consisting of: a ribosomally-encoded xenobiotic agent, a ribosomally-encoded poison, and a ribosomally-encoded recombinase that excises an essential gene for viability.
